(19) United States
(12) Patent Application Publication
(10) Pub. No.: US 2003/0119771 A1
(43) Pub. Date: Jun. 26, 2003

(54) Modulators of bone homeostasis identified in a high-throughput screen

(76) Inventors: Luc Van Rompaey, Keerbergen (BE); Helmuth Hendrikus Gerardus Van Es, Haarlem (NL); Peter Herwig Maria Tomme, Gent (BE); Hubertus Johannes Matheus Klaassen, Herent (BE)

Correspondence Address: SYNNESTVEDT & LECHNER, LLP, 2600 ARAMARK TOWER, 1101 MARKET STREET, PHILADELPHIA, PA, 191072950

(21) Appl. No.: 10/225,630

(22) Filed: Aug. 22, 2002

Related U.S. Application Data
(60) Provisional application No. 60/314,056, filed on Aug. 22, 2001.
(60) Provisional application No. 60/356,935, filed on Feb. 14, 2002.

(51) Int. Cl. A61K 48/00; C12Q 1/68; C07H 21/04; C12P 21/02; C12N 5/08; C12N 9/64; C07K 14/47

(52) U.S. Cl. 514/44; 435/6; 435/69.1; 435/320.1; 435/366; 435/226; 530/350; 536/23.2

(57) ABSTRACT

The invention relates to the field of molecular genetics and medicine. In particular, the present invention relates to the field of functional genomics, i.e., to a method for the identification of genes that function in regulating bone homeostasis, such as the induction of osteogenesis.

In particular, the present invention relates to polynucleotides and the encoded polypeptides that are identified in a high-throughput screen designed to detect modulation of bone alkaline phosphatase activity. Moreover, the present invention relates to vectors, host cells, antibodies and diagnostic methods for detecting diseases involving the discovered polynucleotides, and therapeutic methods for treating such diseases. The invention further relates to methods and means for drug compound screens designed to develop new therapeutic strategies.

### CROSS-REFERENCE TO RELATED APPLICATIONS

This application claims priority from U.S. Provisional Application No. 60/314,056, filed on Aug. 22, 2001, and U.S. Provisional Application No. 60/356,935, filed on Feb. 14, 2002.

### FIELD OF THE INVENTION

The invention relates to the field of molecular genetics and medicine. In particular, the present invention relates to the field of functional genomics, more specifically to newly identified polynucleotides and the polypeptides encoded by these polynucleotides that play a role in bone homeostasis. Furthermore, the invention also relates to the development of a high-throughput screening methodology designed to find the polynucleotides and polypeptides that play a role in bone homeostasis.

### BACKGROUND OF THE INVENTION

Biomedical research is entering a new era now that the sequence of the human genome is almost completely known. Many predicted open reading frames (ORFs) can be extracted from the genome sequence, but definitely not every single ORF, let alone splice variants. The search for novel protein functions is the next logical step in the quest for a complete understanding of the biology of the cell and the organism. This knowledge then allows rational drug development in order to treat diseases.

Functional genomics is a powerful and unbiased approach to discover new genes and the activity that is displayed by the encoded protein. Functional genomics entails screening of cDNA expression libraries in cellular assays where the increase or decrease of a measurable property is read. The present invention provides the methods and means for the identification of nucleic acid compounds, isolated in a high throughput screening assay, that have a function in bone homeostasis.

Osteoblast Differentiation:

Bone contains two distinct cell types, osteoblasts, or bone-forming cells, and osteoclasts, or bone-resorbing cells. To carry out its function, bone is continuously destroyed (resorbed) and rebuilt by an intricate interplay between those two types of cells. For osteoclasts, a detailed cascade of transcription factors and growth factors involved in the progression from progenitor cell to functional osteoclast is well established (Karsenty, 1999; Teitelbaum, 2000). In contrast, very little is known about the osteoblast lineage. During embryonic development, osteoblast differentiation or “bone formation” can occur through two distinct pathways: endochondral or intramembranous ossification. During growth and adulthood, osteoblasts are mainly involved in bone repair and remodelling. It is also known that cells from the osteoblast lineage synthesize and secrete molecules that in turn initiate and control osteoclast differentiation. Osteoblasts are of mesenchymal origin and in cell culture are almost indistinguishable from fibroblasts. Osteoblast differentiation occurs when progenitor cells differentiate into osteoblasts and is characterized by upregulation of endogenous bone alkaline phosphatase activity (BAP). Only two osteoblast-specific transcripts have been identified thus far: one encoding the transcription factor Core-binding factor 1 (Cbfa1), and the other encoding osteocalcin, a secreted molecule that inhibits osteoblast function. Cbfa1 is the master regulator of osteoblast differentiation. Other transcription factors that regulate bone formation and/or Cbfa1 expression have been identified from in vitro and in vivo studies (FIG. 1). For example, Cbfa1 is regulated by different transcription factors like Msx2, Bapx1 or Hoxa1 or by secreted factors like Indian hedgehog (Ihh) or bone morphogenic protein 2 (BMP2). Given the variety and the importance of the biological processes in which these two cell types participate during development and in postnatal life, there is a need to understand their differentiation and function (Karsenty, 1999; Teitelbaum, 2000).

A number of diseases are caused by a disturbance of the fine-tuned balance between bone resorption and bone build-up. More precisely, increases or decreases of osteoclast activity or increases or decreases of osteoblast proliferation and differentiation result in a variety of diseases. The single most important bone disease is osteoporosis, but a number of other diseases represent a large number of patients: hypercalcemia of malignancy, Paget's disease, inflammatory bone diseases like rheumatoid arthritis and periodontal disease, focal osteogenesis occurring during skeletal metastases, Crouzon's syndrome, rickets, opsismodysplasia, pycnodysostosis/Toulouse-Lautrec disease, osteogenesis imperfecta (Karsenty, 1999; Teitelbaum, 2000; Rodan and Martin, 2000; Goltzman, 2001; Karsenty, 2001).

Tissue Engineering/Bone Repair:

Bone remodelling relies on an equilibrium between an anabolic—osteogenic—and a catabolic—bone resorption—process. After bone fractures, bone remodelling processes are required to heal the fracture. However, in many instances, patients are encountered with poorly healing fractures. These patients tend to be older people as well as osteoporotic patients. A surgical intervention is then required to accelerate the recovery. Prostheses can be implanted with or without bone grafting procedures. In some cases where the bone is too porous or where previous implants failed to be incorporated into the bone, current medical practices can offer little or no help. Here, the following procedure could be helpful (Service, 2000; U.S. Pat. No. 6,152,964): mesenchymal progenitor cells are isolated from the patient bone marrow, induced to differentiate into osteoblasts in vitro, mixed with a biopolymer that is used to cover the implant and the implant covered with the cell-biopolymer mixture is then implanted. This procedure offers several advantages over existing procedures: the cement alleviates the need to use bone chips, prepared from the patient's bones. The latter procedure is often painful to the patient. Secondly, the differentiated cells will immediately help to integrate the implant with the surrounding bone, as the cells are osteoblasts. Thirdly, this procedure carries no risk of immunological rejection of the implant, as the patient's own cells are used.

It is therefore important that factors are found that can induce osteoblast differentiation in vitro, starting from pluripotent bone marrow mesenchymal progenitor cells or even from totipotent stem cells.

Currently, a very limited number of compounds have been identified that are able to induce osteoblast differentiation in vitro, e.g., dexamethasone or by recombinant human secreted proteins, e.g., BMP-2 or BMP-7 (Service, 2000).

Therefore, there is a huge interest in finding new human secreted proteins that specifically induce differentiation of osteoblasts or of other cell types involved in bone homeostasis, which can be used in ex vivo/in vitro differentiation programs.

However, research in this area leading to new drug development has been hindered by the lack of high throughput screening (HTS) assays.

Current in vitro assay systems relating to bone homeostasis are cumbersome, time consuming, often based on in vivo systems and certainly not apt for automation. However, because of the complexity of bone homeostasis, it is never used in high throughput screening. Despite the long felt need, in vitro assays relating to bone homeostasis have been performed in 24-, 48- or at most 96-well plates.

To date, efforts to improve HTS have focused on making wells smaller (miniaturization). As one reduces the well size, one can increase the number of wells on each plate to provide more parallel testing. Further, by decreasing the assay volumes, one also decreases the cost of reagents per well. Moreover, because one can run more parallel tests with smaller assay volumes, one can simultaneously test more compounds to find drug candidates. However, miniaturization has inherent costs and complexities. In addition, the use of smaller wells allows the use of primary cells, which are limited in availability.

These costs and complexities relate directly with the three primary components of miniaturizing a screening format. First, one must be able to make the test containers smaller. Secondly, one must be able to accurately dispense all of the necessary assay reagents into more and smaller wells (usually accomplished by liquid handling robots that simultaneously dispense the reagents into many wells). Third, one must be able to “read” the results of the tests in the high-density array.

Given the requirements of paralleled independent assays, each component provides challenges and limits to how much miniaturization is feasible or cost effective. For example, a smaller sample size increases the statistical variability from sample to sample because of the inherent inaccuracies in dispensing smaller volumes of reagents and in measuring weaker sample signals.

A particular problem with high throughput screening relates to the efficient introduction of expressible nucleic acids into the target cells. Adenoviral vectors and adenoviruses comprising the expressible nucleic acid are well known for efficiently transducing target cells. Hence, large libraries of expressible nucleic acids have been made in these adenoviral vectors and adenoviruses. However, said vectors and viruses have a limited host cell range.

Adenovirus contains a linear double-stranded DNA molecule of approximately 36000 base pairs. It contains identical Inverted Terminal Repeats (ITR) of approximately 90-140 base pairs with the exact length depending on the serotype. The viral origins of replication are within the ITRs exactly at the genome ends. The transcription units are divided into early and late regions. Complex splicing and poly-adenylation mechanisms give rise to more than 12 RNA species coding for core proteins, capsid proteins (penton, hexon, fiber and associated proteins), viral protease and proteins necessary for the assembly of the capsid and shutdown of host protein translation (Imperiale et al, 1995).

The interaction of the adenovirus with the host cell occurs via interaction of the knob region of the protruding fiber with a cellular receptor. A receptor for Ad2, Ad5 and probably more adenoviruses, is known as the ‘Coxsackievirus and Adenovirus Receptor’ or CAR protein (Bergelson et al, 1997). The initial step for successful infection is binding of adenovirus to its target cell, a process mediated through the fiber protein. The fiber protein has a trimeric structure (Stouten et al, 1992) with different lengths depending on the virus serotype (Signas et al, 1985; Kidd et al, 1993). Different serotypes have polypeptides with structurally similar N- and C-termini, but different middle stem regions. Although the knob contains some conserved regions between serotypes, the knob proteins show a high degree of variability, indicating that different adenovirus receptors exist. At present, six different subgroups of human adenoviruses have been proposed which in total encompass approximately 50 distinct adenovirus serotypes. A serotype is defined on the basis of its immunological distinctiveness as determined by quantitative neutralization with animal antiserum (horse, rabbit). Besides differences towards the sensitivity against neutralizing antibodies of different adenovirus serotypes, adenoviruses in subgroup C such as Ad2 and Ad5 bind to different receptors as compared to adenoviruses from subgroup B such as Ad3 and Ad7 (Defer et al, 1990; Gall et al, 1996). These serotypes differ in at least proteins responsible for cell binding (fiber protein). However, the fact that (at least some) members of these subgroups are able to bind CAR does not exclude that these viruses have different infection efficiencies in various cell types. Thus, despite their shared ability to bind CAR, differences in the length of the fiber, knob sequence and other capsid proteins e.g., penton base, of the different serotypes may determine the efficiency by which an adenovirus infects a certain target cell.

Most adenoviral gene delivery vectors currently used in functional genomics, gene therapy or vaccination are derived from subgroup C adenoviruses Ad2 or Ad5. The vectors have at least a deletion in the E1 region that renders the recombinant virus replication defective. In this region, novel genetic information can then be introduced. It has been demonstrated extensively that recombinant adenoviruses, in particular serotype 5, are suitable for efficient transfer of genes in vivo to the liver, the airway epithelium and solid tumours in animal models and human xenografts in immuno-deficient mice (Bout 1996, 1997; Blaese et al, 1995).

The use of adenoviral vectors in functional genomics includes building gene expression libraries and in vitro and in vivo gene validation with appropriate meaningful cell based assays or animal models for a particular human disease. Gene transfer vectors derived from adenoviruses (adenoviral vectors) have a number of features that make them particularly useful for gene transfer. However, there are still a number of drawbacks associated with the use of adenoviral vectors and adenoviruses. In particular, the serotypes Ad2 and Ad5 are not ideally suited for delivering additional genetic material to organs other than the liver. In vitro or ex vivo gene transfer for functional genomics using standard Ad2 or Ad5 adenoviral vectors and adenoviruses can be very limited in particular cells involved in bone homeostasis.

### SUMMARY OF THE INVENTION

The present invention relates, in one aspect, to a method for the identification of genes that function in bone homeostasis, and in particular, that induce osteoblast differentiation. More particularly, the screening methods of the present invention, by measuring the up regulation of endogenous bone alkaline phosphatase (BAP) activity, identify polynucleotides and polypeptides that function by inducing the differentiation of precursor cells into osteoblasts. Arrayed nucleic acid libraries are used to transduce arrays of human primary mesenchymal progenitor cells (MPCs) to identify those nucleic acids that up regulate endogenous BAP activity in a high-throughput setting.

In another aspect, the present invention relates to polynucleotides and the encoded polypeptides that are identified in the high-throughput BAP screen, and that accelerate the osteoblast differentiation process.

Moreover, the present invention relates also to vectors, host cells, antibodies and diagnostic methods for detecting osteoblast differentiation-related diseases comprising the aforesaid polynucleotides and polypeptides, and therapeutic methods for bone cell differentiation specifically but also for bone remodelling in general. The invention further relates to methods and means for drug compound screens designed to identify compounds that influence bone cell differentiation and bone remodelling.

### DETAILED DESCRIPTION OF THE INVENTION

This method used to identify the polynucleotides and polypeptides of the present invention comprises (i) the generation of an arrayed nucleic acid library, (ii) introducing said nucleic acid library into cells in an arrayed format, and (iii) determining the osteoblast differentiation. In a preferred embodiment, the present invention provides a method for identifying a sample nucleic acid encoding an exogenous polypeptide, which modulates bone homeostasis, and in particular, induces osteoblast differentiation. said method comprising:

(a) providing a library of a multitude of unique expressible sample nucleic acids,

(b) introducing said sample nucleic acid into a eukaryotic host cell, under conditions wherein said exogenous polypeptide is expressed, and

(c) identifying said sample nucleic acid by detecting modulation of bone homeostasis and in particular induction of osteoblast differentiation of said host cell.

It will thus be understood that the term “identifying” relates to the process of recognizing whether a particular sample nucleic acid induces osteoblast differentiation. Thus, the term “identifying” does not relate to the nature or origin of the sample nucleic acid, e.g., whether the sample nucleic acids “identified” in the said method may or may not have been known previously.

The term “modulates” refers to a sample nucleic acid that modulates, changes, or interferes with bone homeostasis, including bone resorption as well as bone build-up. In this respect, it will be understood that either the sample nucleic acid itself or the product encoded by said sample nucleic acid, e.g., mRNA or polypeptide, interferes with the mechanisms involved in bone homeostasis.

The present invention relates preferentially to methods for identifying sample nucleic acids that modulate bone homeostasis. Bone homeostasis relates to the balance between bone resorption and bone build-up. In other words, the balance between increases or decreases of osteoclast activity or increase or decrease of osteoblast proliferation and differentiation. It is thus to be understood, that modulation of bone homeostasis refers also to the establishment of a balance between bone resorption and bone build-up.

The term “osteoblast differentiation” or “bone formation” relates to the up regulation of endogenous bone alkaline phosphatase activity (BAP). BAP is an early marker in the osteoblast differentiation program (Rodan and Harada, 1997). Endogenous BAP activity is measured by methods known in the art. In particular, adding methylumbelliferyl heptaphosphate (MUP) solution (Sigma cat no M3168) to cells, after which the cells are incubated at 37° C. for 30 minutes. If necessary, the reaction is stopped by the addition of a Na₂CO₃ solution. The resulting fluorescence is subsequently measured, possibly on a fluorescence reader (Fluostar, BMG). It is expected that one skilled in the art is able to adapt the BAP-assay according to their needs. In this respect, the term “endogenous” relates to BAP activity natural or innate to the host cell.

The term “induction” refers to a polypeptide that induces, up regulates, or stimulates osteoblast differentiation. In this respect, it is understood that either the sample nucleic acid itself or the product encoded by said sample nucleic acid induces osteoblast differentiation.

It is understood that the present invention in particular relates to methods as described herein, wherein the identification of said sample nucleic acid by detecting induction of osteoblast differentiation of said host cell comprises measurement of endogenous bone alkaline phosphatase (BAP) activity.

The sample nucleic acid according to the present invention can be genomic DNA, cDNA, RNA, previously cloned DNA, genes, ESTs, synthetic oligonucleotides, randomized sequences, antisense nucleic acids, small interfering RNA (siRNA), genetic suppressor elements, ribozymes, DNA encoding mutant zinc fingers, DNA encoding antibody sequences or any combination thereof. The sample nucleic acid may encode a full-length protein, but it might also encode a partial, not full-length, protein or a protein domain. In this respect it will be understood that the term “exogenous polypeptide” refers to the amino acid sequence encoded by the sample nucleic acid.

Preferably, the sample nucleic acid is part of a set or library of sample nucleic acids that are cloned into a vector or corresponding virus (see below), whereby the set or library of sample nucleic acids may comprise between two and 2×10⁷ individual sample nucleic acids, and usually contains between 100 and 1×10⁶ different sample nucleic acids.

By introducing a sample nucleic acid into a cell it is meant that the sample nucleic acid is transfected, infected or transduced into the cell by any method known to those experienced in the art. The nucleic acids can be delivered by any method known in the art, including, but not limited to, calcium-phosphate co-precipitation and direct needle injection. The nucleic acids may be delivered with delivery vehicles such as viral sequences, viral particles, liposome formulations, lipofectin or precipitating agents and the like. Viral vectors that can be used include, but are not limited to, a herpes virus vector, a baculovirus vector, a lentivirus vector, a retrovirus vector, an alphavirus vector, an adeno-associated virus vector or an adenoviral vector, adenovirus or any combination thereof.

The eukaryotic host cells or host cell line into which the adenoviral vector is introduced in a method according to the present invention as defined herein can be any desired cell that can be induced to differentiate to osteoblasts in vitro. These cells can be obtained from any organism including, but not limited to, mammals such as humans, canine and rodents, reptiles, birds, amphibian and fish. The host cells are preferably of mesenchymal origin. Preferably, the eukaryotic host cells are mesenchymal pluripotent cells (MPC), also termed mesenchymal stem cells (MSCs), more preferably, primary MPCS, and even more preferably, human primary MPCs. In this application, the term “mesenchymal” refers to the phenotype of the cells, i.e., cells that can differentiate into osteoblasts, chondrocytes, stromal fibroblasts, myoblasts, neuronal cells, pre/adipocytes and/or tenoblasts, as is well known in the art.

It will thus be understood that the present invention in particular relates to a method as described herein, wherein said eukaryotic host cell is a mesenchymal pluripotent cell (MPC), preferably a primary MPC, and even more preferably a primary human MPC.

Osteoblast differentiation relates to the remodeling of a mesenchymal cell, which goes together with the up regulation of BAP activity. Hence, the term “assaying the phenotype” relates to assessing the ability of a sample nucleic acid to interfere with (modulate) one or more of the mechanisms involved in bone homeostasis by determining a change of phenotype compared to controls, preferably by using the method of the invention. The controls display a particular phenotype in the absence of the modulator. It will thus be understood that also the survival of mesenchymal cells due to the influence of a sample nucleic acid is contemplated in the present invention, if this survival is the consequence of the modulator. Increased survival and/or proliferation lead to confluence of cultures faster. The state of confluence is a boost to the start of most differentiation programs and can work together with introduced sample nucleic acids to start the program of osteoblast differentiation.

By assaying the phenotype of the transduced cells it is meant that each well is scored for a difference in phenotype compared to a control well, and in particular the upregulation of BAP activity compared to a control well. The set up of controls are well known in the art, erg., in general a control well will differ in only one parameter from the well under investigation. The scoring can be done visually by light microscopy or by fluorescent microscopy after staining of the cells with a fluorescent marker, as well as by measuring for example luminescence, absorbance of light, fluorescence, and/or FRET. All of the aforementioned techniques usable for scoring are well known in the art. It will furthermore be appreciated by one skilled in the art, that said techniques can be modified according to specific needs. For high throughput screening, automated data capture and image analyzers are preferred.

The generation of a recombinant adenoviral vector library containing the sample nucleic acids, and the production of active recombinant adenoviruses by introducing the viral vectors—containing the sample nucleic acid—in their respective packaging cell lines such as the PER.C6 or PER.C6/E2A packaging cell lines (U.S. Pat. No. 5,994,128, WO99/64582), are all performed in a high-throughput setting basically as described in WO99/64582 and further outlined in the examples. Hence, the adenovirus library is directly derived from and comparable to the adenoviral vector library. Both the adenoviral vector library and the adenovirus library contain a multitude of unique expressible sample nucleic acids. Each library consisting essentially of one or more adenoviral vector(s) or adenoviruses of a first type comprising at least one unique nucleic acid in an aqueous medium. It will be appreciated that both the adenoviral vector library as well as the adenovirus library can be used in a method according to the invention.

Accordingly, the present invention relates to a method according to the present invention, wherein said library consists essentially of one or more adenoviral vector(s) or adenoviruses of a first type comprising at least one unique nucleic acid, in an aqueous medium.

It will be understood that the present invention relates to a method according to the present invention, wherein said library includes a multiplicity of compartments, each of said compartments consisting essentially of one or more adenoviral vector(s) or adenoviruses of a first type comprising at least one unique nucleic acid, in an aqueous medium.

To identify and assign function to the product(s) encoded by the sample nucleic acids, the recombinant adenoviral vectors or adenoviruses that express the product(s) of the sample nucleic acids, e.g., RNA or polypeptides, are introduced into the host cell in a high-throughput setting.

The use of adenoviral vectors or adenoviruses in functional genomics includes building gene expression libraries and in vitro and in vivo gene validation with appropriate meaningful cell based assays or animal models for a particular human disease. Gene transfer vectors derived from adenoviruses (adenoviral vectors) have a number of features that make them particularly useful for gene transfer. However, there are still a number of drawbacks associated with the use of adenoviral vectors or adenoviruses. In particular, the serotypes Ad2 and Ad5 are not ideally suited for delivering additional genetic material to organs other than the liver. In vitro or ex vivogene transfer for functional genomics using standard Ad2 or Ad5 adenoviral vectors or adenoviruses can be very limited in particular if said adenoviral vectors or adenoviruses are used in cells involved in bone homeostasis. The present invention demonstrates that by either increasing the number of cellular receptors or by introducing specific receptors for a serotype enhances the transduction efficiency remarkably. In this respect, also increasing the number of “redundant” receptors significantly enhances the transduction efficiency. Since various adenoviral vector or adenoviruses libraries are based on Ad2 or Ad5 viruses, the CAR receptor for Ad2 and Ad5 is particularly useful. The present invention thus relates to a method for introducing receptors into a host cell recognizing the fiber protein of the adenoviral vector or adenoviruses in which said library has been generated. Similarly, the present invention relates to a method for enhancing the number of receptors into a host cell recognizing the fiber protein of the adenoviral vector or adenoviruses in which said library has been generated. Enhancing the number of receptors can, for example, be established by introducing nucleic acids encoding for said receptor. Introduction of the receptor can be according to the means as described herein. The present invention relates specifically to introducing the receptor by an adenoviral vector or adenovirus of a second type, i.e., different from the adenoviral vector that comprises the sample nucleic acids. It will be clear that adenoviral vector or adenovirus of a first type refers to an adenoviral vector or adenoviruses into which said sample nucleic acid library has been generated, comprising a multitude of unique expressible nucleic acids. Similarly, it will be clear that adenoviral vector or adenovirus of a second type refers to an adenoviral vector or adenovirus containing a nucleic acid encoding specifically for a receptor for a fiber protein. Hence, the terms “first type” or “second type” do not relate to a specific fiber protein. It has been demonstrated that the combination of CAR receptor in the adenoviral vector or adenovirus of the second type with Ad5fib5 in the adenoviral vector or adenovirus of the first type is particularly useful in this respect.

Therefore, the present invention in particular relates to a method for introducing an adenoviral vector or adenovirus of a first type into a host cell to enhance the number of host cell receptor(s) for said adenovirus or adenoviral vector of the first type, said method comprising:

(a) providing an adenoviral vector or adenovirus of a second type, comprising a gene encoding a host cell receptor for the fiber protein of the adenoviral vector or adenovirus of the first type,

(b) introducing an adenoviral vector or adenovirus of the second type into said host cells, under conditions allowing for expression of said host cell receptor,

(c) providing an adenoviral vector or adenovirus of the first type, said adenoviral vector or adenovirus of the first type comprising a sample nucleic acid, and

(d) introducing the adenoviral vector or adenovirus of the first type into the host cell of step (b) via binding of the fiber protein of the adenovirus of the first type to the host cell receptor provided by the adenoviral vector or adenovirus of the second type.

It will thus be contemplated that the present invention in particular relates to a method for according to the present invention, wherein said method is preceded by the following steps:

(a) providing an adenoviral vector or adenovirus of a second type, comprising a nucleic acid sequence encoding a cell receptor capable of interacting with the fiber protein of the adenoviral vector or adenovirus of the first type, and,

(b) introducing said adenoviral vector or adenovirus of the second type into said eukaryotic host cells, under conditions allowing for expression of said cell receptor in said host cell.

High Throughput Screening

In view of the complexity, the potential number of factors, and the intricate relations between these factors that are involved in the maintenance of bone homeostasis, and in particular osteoblast differentiation, it is preferred to screen for a large number of factors. Therefore, the measurement of the function of the sample nucleic acids, which in the set-up of the present invention is defined as a modulation of bone homeostasis and in particular osteoblast differentiation, is preferably performed in a high-throughput and/or miniaturized set-up.

As used herein, the terms “high-throughput screen” or “HTS” refer to an assay involving the exposure of a known (or target) to a group (or library) of unknowns (or test compounds) in an automated fashion, wherein the progression of a reaction associated with the target is assayed for, the results of which correlate to the level of biological activity of a particular test compound. This target-associated reaction often involves binding, such as ligand to receptor. The HTS describes a method where many discrete compounds are tested in parallel so that large numbers of test compounds are screened for biological activity simultaneously or nearly simultaneously, with the capacity for robotic manipulation, and small sample volume.

Currently, the most widely established techniques utilize 96-well microtitre plates. In this format, 96 independent tests are performed simultaneously on a single 8 cm×12 cm plastic plate that contains 96 reaction wells. These wells typically require assay volumes that range from 50 to 500 μl addition to the plates, many instruments, materials, pipettors, robotics, plate washers and plate readers are commercially available to fit the 96-well format to a wide range of homogeneous and heterogeneous assays. The present invention particularly relates to a 96-wells, 384-wells or larger setting.

Hence, the present invention relates to a method as described herein, wherein at least one step, such as, for example the introducing step, but more preferentially two or more steps are performed in a miniaturized and/or high-throughput format.

In a further aspect of the present invention, methods are provided to validate whether the candidate sample nucleic acid from a primary screen is a bona fide modulator of bone homeostasis, and in particular osteoblast differentiation. Hence, the present invention relates to a method according to the present invention further comprising a validation step in which said sample nucleic acid is further validated for inducing transcription of ALPL (human alkaline phosphatase liver/bone/kidney), ALPI (human alkaline phosphatase intestinal), ALPP (human alkaline phosphatase placental), and/or ALPP2 (human alkaline phosphatase placental-like). Assays to determine the induction of transcription are well known in the art, and encompass, but are not limited, to Northern blot analysis or quantitative RT-PCR of ALPL, ALPI, ALPP, and/or ALPP2 mRNAs.

It will be appreciated by one skilled in the art, that alternative forms of validation assays might exist, or that validation assays might be adapted according to specific needs. It is believed that these variations to the assays are within the skill of the artisan. Hence, alternative assays, such as described in Examples 10, 11, 12, 13, as well as variation to the herein described validation assays are pertained in the present invention.

Identified Modulators, and the Applications Thereof.

The method according to the invention results in the identification of modulators or sample nucleic acids that are able to interfere with bone homeostasis, and in particular, induce osteoblast differentiation. The modulation of bone homeostasis or the differentiation into osteoblasts can result from either the sample nucleic acid itself or the product encoded by said sample nucleic acid. One aspect of the present invention provides any nucleic acid identifiable by the methods according to the present invention.

The invention further provides the sequence identities (SEQ ID NOS: 1, 3, 5, 7, and 9) of the group of nucleic acids and corresponding polypeptides (SEQ ID NOS: 2, 4, 6, 8, and 10) that modulate bone homeostasis or induce osteoblast differentiation in a primary screen, and in validation assays.

In a preferred embodiment, the present invention provides an isolated nucleic acid selected from a group of nucleic acids identifiable as inducers of osteoblast differentiation consisting of:

(a) a DNA sequence of SEQ ID NO: 1, 3, 5, 7, or 9 or the complement thereof,

(b) a RNA sequence corresponding to SEQ ID NO: 1, 3, 5, 7, or 9 or the complement thereof,

(c) a nucleic acid specifically hybridizing to one of the nucleotide sequences defined in (a) or (b),

(d) a nucleic acid having a nucleotide sequence which is at least 65% identical to a sequence defined in (a),

(e) a nucleic acid encoding a protein with an amino acid sequence which is at least 65% identical to the amino acid sequence of SEQ ID NO: 2, 4, 6, 8, or 10,

(f) a nucleic acid encoding a protein comprising the amino acid sequence of SEQ ID NO: 2, 4, 6, 8, or 10,

(g) a nucleic acid that is degenerated as a result of the genetic code to a nucleotide sequence of SEQ ID NO: 1, 3, 5, 7, or 9 or as defined in (a) to (f),

(h) a nucleic acid that is diverged due to differences in codon usage between organisms to a nucleotide sequence encoding a protein of SEQ ID NO: 2, 4, 6, 8, or 10, or as defined in (a) to (g),

(i) a nucleic acid that is diverged due to the differences between alleles encoding a protein as given in SEQ ID NO: 2, 4, 6, 8, or 10, or as defined in (a) to (h),

(j) a nucleic acid encoding an immunologically active and/or functional fragment of a protein encoded by a DNA sequence of SEQ ID NO: 1, 3, 5, 7, or 9,

(k) a nucleic acid encoding a gene family member of the nucleic acid of SEQ ID NO: 1, 3, 5, 7, or 9, and

(l) a nucleic acid encoding a protein as defined in SEQ ID NO: 2, 4, 6, 8, or 10, or a nucleic acid as defined in any one of (a) to (k) wherein said sequence is DNA, cDNA, genomic DNA or synthetic DNA.

It is well recognized that the genetic code is degenerate, i.e., an amino acid may be coded for by more than one codon. Degenerate codons encode the same amino acid residue, but contain different triplets of nucleotides. Accordingly, for a given polynucleotide sequence encoding a particular modulator of bone homeostasis, there will be many degenerate polynucleotide sequences encoding that modulator. These degenerate polynucleotide sequences are considered within the scope of this invention.

Furthermore, it will also be appreciated by one of skill in the art that different organisms, cells, and cellular compartments may utilize different genetic codes. Thus, a single polynucleotide sequence may encode different polypeptides depending on its cellular context. Accordingly, in addition to the standard genetic code, polypeptides encoded by non-standard genetic codes are also considered within the scope of this invention. These non-standard genetic codes include, but are not limited to, the vertebrate mitochondrial code, the yeast mitochondrial code, the mold, protozoan, and coelenterate mitochondrial code, the mycoplasma/spiroplasma code, the invertebrate mitochondrial code, the ciliate, dasycladacean and hexamita nuclear code, the echinoderm mitochondrial code, the euplotid nuclear code, the bacterial and plant plastid code, the alternative yeast nuclear code, the ascidian mitochondrial code, the flatworm mitochondrial code, blepharisma nuclear code, chlorophycean mitochondrial code, trematode mitochondrial code, scenedesmus obliquus mitochondrial code, and the thraustochytrium mitochondrial code.

The term “immunologically active” is understood to mean that a molecule or specific fragments thereof, such as epitopes or haptens, are recognized or, in other words, are bound by antibodies.

In a preferred embodiment, the invention provides a nucleic acid molecule of at least 10 nucleotides in length specifically hybridizing with any of the nucleic acids of the present invention. In particular, longer nucleic acid molecules are contemplated, i.e., of about 15, 20, 25, 30, 40, 50, 75, 100, 200 or even more nucleotides. It is to be understood that also shorter probes may be useful (having for instance 10, 11, 12, 13 or 14 nucleotides). Different types of hybridization techniques and formats are well known in the art. The nucleic acid molecule may be labelled with, for example, a radioactive isotope or an immunofluorescent compound, thereby allowing the detection of the hybrid. As such, the present invention provides methods for detecting the nucleic acids of the present invention.

In a further embodiment, the invention provides a nucleic acid molecule of at least 15 nucleotides in length as described above, wherein said nucleic acid molecule is liable to act as a primer for specifically amplifying a nucleic acid of the present invention, or a part thereof. It is to be understood that said primers can be shorter, e.g., 10, 11, 12, 13, or 14 nucleotides, or longer, e.g., 16, 17, 18, 19, 20, 25, or 30 nucleotides.

Sets of said primers may be used in any well described amplification technique known in the art such as Polymerase Chain Reaction (PCR), Transcription Mediated Amplification (TMA), or Nucleic Acid Sequence Based Amplification (NASBA) techniques, thereby allowing the amplification and subsequent detection of the nucleic acid of the present invention. Preferably, said primers may also be used to specifically amplify the nucleic acids of the present invention. As such, the present invention provides methods for detecting the nucleic acids of the present invention.

The present invention is also directed to variants of the nucleotide sequence of the nucleic acid disclosed in SEQ ID NO: 1, 3, 5, 7, or 9 or the corresponding complementary strand.

The present invention is also directed to nucleic acid molecules which comprise, or alternatively consist of, a nucleotide sequence which is at least 65%, 70%, 75%, 80%, 85%, 90%, 95%, 96%, 97%, 97.5%, 98%, 98.5%, 99% or 99.5% identical to the nucleotide sequences as represented in SEQ ID NO: 1, 3, 5, 7, or 9, or the corresponding complementary strand, or parts thereof. Said parts are preferably unique parts.

By a nucleic acid having a nucleotide sequence at least, for example, 95% “identity” to a reference nucleotide sequence of the present invention, it is intended that the nucleotide sequence of said nucleic acid is identical to the reference sequence except that the nucleotide sequence may include up to five point mutations per each 100 nucleotides of the reference nucleotide sequence. In other words, to obtain a nucleic acid having a nucleotide sequence of at least 95% identity to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. As a practical matter, whether any particular nucleic acid molecule is at least 65%, 70%, 75%, 80%, 85%, 90%, 95%, 96%, 97%, 97.5%, 98%, 98.5%, 99% or 99.5% identical to a nucleotide sequence of the present invention can be determined using known algorithms. A preferred method for determining the best overall match between a query sequence (a sequence of the present invention) and a subject sequence can be determined using a Blast search (Altschul et al., 1997).

Nucleic acids which specifically hybridize to any of the strands of the nucleic acid molecules of the present invention as specified under SEQ ID NO: 1, 3, 5, 7, or 9 under stringent hybridization conditions or lower stringency conditions are also particularly encompassed by the present invention. “Stringent hybridization conditions” refers to an overnight incubation at 68° C. in a solution comprising 5×SSC (750 mM NaCl, 75 mM trisodium citrate), 50 mM sodium phosphate (pH 7.6), 5×Denhardt's solution, 10% dextran sulfate and 20 μg/ml denatured sheared salmon sperm DNA, followed by washing the filters in 0.1×SSC at about 65° C. Changes in the stringency of hybridization are primarily accomplished through the manipulation of the SSC dilution in the washing steps (higher concentration SSC in washing buffer results in lower stringency) and the temperature (lower washing temperature results in lower stringency). For example, lower stringency conditions include washes performed at 1×SSC and at 55-60° C. Hybridization under high and low stringency conditions are principles which are well understood by the person skilled in the art (see, for instance, Sambrook et al. Molecular Cloning: A laboratory manual. Cold Spring Harbor laboratory press 1989).

The nucleotide sequences presented in the present invention may be extended utilizing a partial nucleotide sequence and employing various methods known in the art to detect the full sequence in case said sequence would only be a part of a coding region as well as upstream sequences such as promoters and regulatory elements.

In addition, some of the proteins identified herein that play a role in modulation of bone homeostasis and in particular the induction of osteoblast differentiation, could serve as specific markers for the pathological process. Therefore, they can be used as diagnostic markers or as therapeutic markers that are used to target specific drugs towards the pathological tissue.

Methods which are well known to those skilled in the art may be used to construct expression vectors containing at least a fragment of the nucleic acids of the present invention together with appropriate transcriptional and translational control elements. These methods include in vitro recombinant DNA techniques, synthetic techniques, and in vivo genetic recombination. Such techniques are described, for example, in Sambrook et al. Molecular Cloning: A laboratory manual. Cold Spring Harbor laboratory press 1989.

The present invention relates also to vectors comprising the sample nucleic acid of the present invention. The present invention particularly contemplates recombinant expression vectors, said vectors comprising a vector sequence, an appropriate prokaryotic, eukaryotic or viral or synthetic promoter sequence followed by the sample nucleic acid of the present invention. Preferably, the vector used for expressing the sample nucleic acid according to the present invention can be a vector for expression in E. coli, a yeast shuttle vector, or a yeast two-hybrid vector, a plant vector, an insect vector, a mammalian expression vector, including but not limited to, a herpes virus vector, a baculovirus vector, a lentivirus vector, a retrovirus vector, an alphavirus vector, an adenoviral vector or any combination thereof.

In a preferred embodiment, the invention provides a vector comprising a nucleic acid sequence of the present invention. As such, said nucleic acid is a member selected from a group of nucleic acids identifiable as modulators of bone homeostasis and in particular as inducers of osteoblast differentiation. Preferably, said nucleic acid is a member selected from a group represented by SEQ ID NOS: 1, 3, 5, 7, or 9 or variants, fragments or homologues thereof.

In a preferred embodiment said vector is an expression vector wherein the nucleotide sequence is operably linked to one or more control sequences allowing the expression of said sequence in prokaryotic and/or eukaryotic host cells.

In a preferred embodiment said vector is an adenoviral vector.

In a preferred embodiment said vector is generated from an adenoviral adapter vector which contains the left ITR and part of the E2B region, and in which the E1 region has been exchanged for a mammalian promoter, a polylinker sequence, and a poly-adenylation signal.

As will be understood by those skilled in the art, it may be advantageous to produce products encoded by the aforementioned isolated nucleotide sequences possessing non-naturally occurring codons. For example, codons preferred by a particular prokaryotic or eukaryotic host can be selected to increase the rate of protein expression or to produce an RNA transcript having desirable properties, such as a longer half-life, thereby increasing the amount of expressable polypeptides in a cell, which may be desirable for multiple applications.

The nucleotide sequences of the present invention can be engineered using methods generally known in the art in order to alter protein encoding sequences for a variety of reasons, including but not limited to, alterations which modify the cloning, processing, and/or expression of the gene product.

DNA shuffling by random fragmentation or PCR reassembly of gene fragments or synthetic oligonucleotides may be used to engineer the nucleotide sequences. For example, site-directed mutagenesis may be used to insert new restriction sites, alter glycosylation patterns, change codon preference, produce splice variants, introduce mutations, and so forth.

Furthermore, natural, modified, or recombinant nucleotide sequences may be ligated to partial or complete nucleic acid sequences of the present invention to encode a fusion protein. For example, to screen peptide libraries for inhibitors of the product of the nucleic acids of the present invention, it may be useful to encode a chimeric protein that can be recognized by a commercially available antibody. A fusion protein may also be engineered to contain a cleavage site located between the protein coding sequence and the heterologous protein sequence, so that the protein may be cleaved and purified away from the heterologous moiety. A fusion protein can also be generated to simplify purification of the protein. For example, multiple histidines, glutathione-S-transferase, or an epitope tag can be fused to the protein. Passage of such a fusion protein onto a nickel, glutathione, or antibody column would result in removal of most impurities.

In a further embodiment, the invention provides a host cell containing an integrated or episomal copy of any of the nucleotide sequences of the present invention or any functional parts thereof. In a more preferred embodiment, the invention provides a host cell containing a vector comprising a nucleic acid sequence according to the present invention.

The latter host cell can be obtained from any organism including, but not limited to, mammals, such as humans, canines and rodents, amphibia, reptiles, birds, fish, nematodes, yeast, fungi, bacteria, insects and plants.

In this regard, the term “functional parts” refers to any part of the nucleotide sequence of the present invention which exhibits substantially a similar, but not necessarily identical, activity as the complete nucleotide sequence, i.e., is able to modulate bone homeostasis and in particular induce osteoblast differentiation, both in vitro and in vivo.

In a preferred embodiment, the invention provides an isolated polypeptide encodable by any of the herein mentioned nucleic acids, or a variant or a derivative thereof, or an immunologically active and/or functional fragment thereof.

It is thus understood that the present invention relates to a polypeptide having an amino acid sequence as given in SEQ ID NO: 2, 4, 6, 8, or 10, or a variant or a derivative thereof, or an immunologically active and/or functional fragment thereof.

“Variants” of a protein of the invention are those peptides, oligopeptides, polypeptides, proteins and enzymes which contain amino acid substitutions, deletions and/or additions relative to the said protein with respect to which they are a homologue, while maintaining the ability to modulate bone homeostasis and in particular induce osteoblast differentiation. In other words, the term “variant” refers to a polypeptide or protein differing from the polypeptide or protein of the present invention, but retaining essential properties thereof, i.e., modulation of bone homeostasis and in particular the induction of osteoblast differentiation. It will be understood that modulation of bone homeostasis and in particular the induction of osteoblast differentiation by these variants relates to inhibition as well as enhancing osteoblast and/or osteoclast activity, irrespective of the functional activity of the protein the variant relates to. The present invention thus particularly contemplates dominant-negatives as well as dominant positives, and related polypeptides and proteins. In the present invention, the functional activity of a protein relates to the “function”, which refers to the ability per se to modulate bone homeostasis and in particular to induce osteoblast differentiation (a qualitative measure), and to the “activity” which refers to the amount of this ability to modulate bone homeostasis and in particular to induce osteoblast differentiation per molecule (a quantitative measure). Generally, variants are overall closely similar, and, in many regions, identical to the polypeptide or protein of the present invention. For example, a homologue of said protein will consist of a bioactive amino acid sequence variant of said protein. To produce such homologues, amino acids present in the said protein can be replaced by other amino acids having similar properties, for example hydrophobicity, hydrophilicity, hydrophobic moment, antigenicity, propensity to form or break α-helical structures or β-sheet structures, and so on. Amino acid substitutions are typically of single residues, but may be clustered depending upon functional constraints placed upon the polypeptide; insertions will usually be of the order of about 1-10 amino acid residues and deletions will range from about 1-20 residues. Preferably, amino acid substitutions will comprise conservative amino acid substitutions. It will be understood by one skilled in the art, that said variants could easily be tested for their ability to modulate bone homeostasis and in particular to induce osteoblast differentiation in any of the assays described in the present invention.

Insertional amino acid sequence variants of a protein of the invention are those in which one or more amino acid residues are introduced into a predetermined site in said protein. Insertions can comprise amino terminal and/or carboxyl-terminal fusions as well as intra-sequence insertions of single or multiple amino acids. Generally, insertions within the amino acid sequence will be smaller than amino or carboxyl terminal fusions, of the order of about 1 to 10 residues. Deletion variants of a protein of the invention are characterized by the removal of one or more amino acids from the amino acid sequence of said protein.

Amino acid variants of a protein of the invention may readily be made using peptide synthetic techniques well known in the art, such as solid phase peptide synthesis and the like, or by recombinant DNA manipulations. The manipulation of DNA sequences to produce variant proteins, which manifest as substitution, insertion or deletion variants are well known in the art.

“Derivatives” of a protein of the invention are those peptides, oligopeptides, polypeptides, proteins and enzymes which comprise at least about 5 contiguous amino acid residues of said polypeptide but which retain the biological activity of said protein. Preferably said derivatives will comprise at least 6, 7, 8, 9, 10, 11, 12, 13, 14, or 15 contiguous amino acid residues of said protein. A “derivative” may further comprise additional naturally-occurring, altered glycosylated, acylated or non-naturally occurring amino acid residues compared to the amino acid sequence of a naturally-occurring form of said polypeptide. Alternatively or in addition, a derivative may comprise one or more non-amino acid substituents compared to the amino acid sequence of a naturally-occurring form of said polypeptide, for example a reporter molecule or other ligand, covalently or non-covalently bound to the amino acid sequence such as, for example, a reporter molecule to facilitate its detection.

In the context of the current invention are embodied homologues, derivatives and/or immunologically active fragments of any of the newly identified sequences that modulate bone homeostasis and in particular induce osteoblast differentiation, as defined above.

The term “homologue” relates to the molecule in a non-human species, that corresponds to the molecule of the present invention, i.e., able to modulate bone homeostasis and in particular induce osteoblast differentiation, as measured in the method of the invention, with or without dose dependency.

In a preferred embodiment, the invention provides a method for producing the polypeptide of the present invention, the method comprising culturing host cells comprising a nucleic acid of the invention as defined above under conditions allowing the expression of the polypeptide and recovering the produced polypeptide from the culture. Alternative methods for producing said polypeptides of the invention are well known in the art, such as, for example, chemical synthesis.

The present invention is also directed to polypeptides, which comprise, or alternatively consist of, an amino acid sequence which is at least 65%, 70%, 75%, 80%, 85%, 90%, 95%, 96%, 97%, 97.5%, 98%, 98.5%, 99% or 99.5% identical to the amino acid sequences of the present invention, wherein said amino acid sequence of the invention, the so-called reference sequence, is at least 30 amino acids in length. However, for reference sequences smaller than 30 amino acids the polypeptide must consist of an amino acid sequence which is at least 65%, 70%, 75%, 80%, 85%, 90%, 95%, 96%, 97%, 97.5%, 98%, 98.5%, 99% or 99.5% identical to the reference sequence.

By a polypeptide having an amino acid sequence of at least, for example, 95% “identity” to a reference amino acid sequence of the present invention, it is intended that the amino acid sequence of the polypeptide is identical to the reference sequence except that the amino acid sequence may include up to five amino acid alterations per each 100 amino acids of the reference polypeptide amino acid sequence. In other words, to obtain a polypeptide having an amino acid sequence at least 95% identical to a reference amino acid sequence, up to 5% of the amino acids in the reference sequence may be deleted or substituted with another amino acid, or a number of amino acids up to 5% of the total amino acids in the reference sequence may be inserted into the reference sequence. As a practical matter, whether any particular polypeptide is at least 65%, 70%, 75%, 80%, 85%, 90%, 95%, 96%, 97%, 97.5%, 98%, 98.5%, 99% or 99.5% identical to a polypeptide sequence of the present invention can be determined using known algorithms. A preferred method for determining the best overall match between a query sequence (a sequence of the present invention) and a subject sequence can be determined using BLASTp (Altschul et al., 1997).

It will be understood that the present invention relates to the use of a nucleic acid encoding a protein comprising an amino acid sequence which is at least 65% identical to SEQ ID NO: 2, 4, 6, 8, or 10, or a functional fragment thereof, for modulating bone homeostasis, and in particular inducing osteoblast differentiation.

Similarly, the present invention relates to the use of a protein comprising an amino acid sequence which is at least 65% identical to SEQ ID NO: 2, 4, 6, 8, or 10, or a functional fragment thereof, for modulating bone homeostasis, and in particular inducing osteoblast differentiation.

The present invention thus also relates to the use of a nucleic acid comprising a nucleic acid sequence which is at least 65% identical to SEQ ID NO: 1, 3, 5, 7, or 9 or a functional fragment thereof, for modulating bone homeostasis, and in particular inducing osteoblast differentiation.

Antibodies

In another preferred embodiment, the invention provides an antibody specifically recognizing the polypeptides of the present invention, or a specific epitope of said polypeptide. The term epitope refers to portions of a polypeptide having antigenic or immunogenic activity in an animal, preferably a mammal, and most preferably in a human. Epitope-bearing polypeptides of the present invention may be used to induce antibodies according to methods well known in the art including, but not limited to, in vivo immunisation, in vitro immunisation, phage display methods or ribosome display.

The antibody of the present invention relates to any polyclonal or monoclonal antibody binding to a protein of the present invention. The term “monoclonal antibody” used herein refers to an antibody composition having a homogeneous antibody population. The term is not limiting regarding the species or source of the antibody, nor is it intended to be limited by the manner in which it is made. Hence, the term “antibody” contemplates also antibodies derived from camels (Arabian and Bactrian), or the genus lama. Thus, the term “antibody” also refers to antibodies derived from phage display technology or drug screening programs. In addition, the term “antibody” also refers to humanized antibodies in which at least a portion of the framework regions of an immunoglobulin are derived from human immunoglobulin sequences and single chain antibodies as described in U.S. Pat. No. 4,946,778 and to fragments of antibodies such as F_{ab}, F_{′(ab)2}, Fᵥ, and other fragments which retain the antigen binding function and specificity of the parent antibody. The term “antibody” also refers to diabodies, triabodies or multimeric (mono-, bi-, tetra- or polyvalent/mono-, bi- or polyspecific) antibodies, as well as enzybodies, i.e., artificial antibodies with enzyme activity. Combinations of antibodies with any other molecule that increases affinity or specificity, are also contemplated within the term “antibody”. Antibodies also include modified forms (e.g., PEGylated or polysialylated form (Fernandes & Gregoriadis, 1997) as well as covalently or non-covalently polymer bound forms. In addition, the term “antibody” also pertains to antibody-mimicking compounds of any nature, such as, for example, derived from lipids, carbohydrates, nucleic acids or analogues e.g., PNA, aptamers (see Jayasena, 1999).

In specific embodiments, antibodies of the present invention cross-react with murine, goat, rat and/or rabbit homologues of human proteins and the corresponding epitopes thereof. Further included in the present invention are antibodies that bind polypeptides encoded by nucleic acids that hybridize to a polynucleotide of the present invention under stringent hybridization conditions (as described herein). As such, the present invention provides a method for detecting the polypeptides of the present invention, the method comprising the use of the antibodies in immunoassays for qualitatively or quantitatively measuring levels of the polypeptides of the present invention in biological samples.

Thus the invention contemplates also a method for detecting a nucleic acid or a polypeptide as described herein, preferably by an antibody of the present invention.

In particular, the present invention relates to an antibody specifically recognizing a polypeptide encodable by a nucleic acid according to the present invention, or a specific epitope of said polypeptide.

Antibodies of the present invention may act as agonists or antagonists of the polypeptides of the present invention. Preferably, antibodies of the present invention bind an antigenic epitope as disclosed herein, or a particular portion of the proteins of the present invention.

Antibodies of the present invention may be used, for example, but not limited to, to purify, detect, target, and/or inhibit the activity of the polypeptides of the present invention, in bone homeostasis and in particular osteoblast differentiation, including both in vitro and in vivo diagnostic and therapeutic methods, as well as in drug screens (see infra).

Antisense Technology

Antisense technology can be used to control gene expression, for example for inhibition of gene expression, i.e., transcription, as described in the art. As such, antisense nucleic acids can be used as antagonist compounds, and may be employed to regulate the effects of the polypeptides of the present invention on the modulation of bone homeostasis and in particular the induction of osteoblast differentiation, both in vitro and in vivo.

Thus, in a further embodiment, the present invention provides a polynucleotide sequence comprising a nucleic acid according to the present invention, encoding the antisense nucleic acid of the invention. Such antisense nucleic acids can be constructed by recombinant DNA technology methods standard in the art.

In a preferred embodiment, the present invention provides a vector comprising a polynucleotide sequence as described herein encoding an antisense nucleic acid.

In a more preferred embodiment, said vector is an expression vector wherein the antisense polynucleotide sequence is operably linked to one or more control sequences allowing the expression, i.e., transcription, of said sequence in prokaryotic and/or eukaryotic host cells.

Potential antagonists according to the invention also include catalytic RNA, or a ribozyme. Ribozymes cleave mRNA at site-specific recognition sequences and can be used to destroy mRNAs corresponding to the nucleic acids of the present invention. The construction and production of ribozymes is well known in the art. As in the antisense approach, ribozymes of the invention can be used as antagonist compounds, and can be delivered to cells to, for example, inhibit in vitro or in vivo osteoclast and/or osteoblast activity, or inhibit inhibitory actions on osteoblast differentiation and activity, and in particular to inhibit or stimulate the induction of osteoblast differentiation effects of the polypeptides of the present invention. Similarly, the nucleic acids of the present invention, the RNA molecules derived thereof, functional equivalent parts or fragments thereof can contain enzymatic activity or can squelch RNA binding polypeptides or can exert effects as antisense agents by binding the endogenous sense strand of mRNA, all of which can modulate bone homeostasis, and preferably, osteoblast induction, and even more preferably upregulation of BAP expression.

Similarly, the nucleic acids of the present invention, the RNA molecules derived thereof, functional equivalent parts or fragments thereof can contain enzymatic activity which can modulate bone homeostasis, and preferably, osteoblast induction, and even more preferably upregulation of BAP expression.

Therefore, the present invention relates to a method for regulating gene transcription or expression comprising:

(a) introducing a polynucleotide sequence comprising a nucleic acid sequence according to the present invention into a host cell,

(b) regulating the expression of a target gene, wherein the polynucleotide sequence regulates the transcription or expression of the target.

In a preferred embodiment, the present invention relates to a method for regulating gene expression, and preferably up-regulating gene expression, comprising:

(a) introducing a polynucleotide sequence comprising a nucleic acid sequence according to the present invention into a host cell,

(b) regulating, and preferably up-regulating the expression of a target gene in said host cell, by said polynucleotide sequence.

Therefore, the present invention relates to a method for modulating bone homeostasis, and preferably, osteoblast differentiation, in particular, induction of osteoblast differentiation, and even more preferably upregulation of BAP expression, comprising introducing a polynucleotide sequence comprising the nucleic acids sequences according to the present invention into a host cell, under conditions which result in expression of the poynucleotide sequence in the host cell, which may in turn result in modulation of bone homeostasis, osteoblast differentiation, and/or upregulation of BAP expression, by said polynucleotide sequences.

The invention further provides the nucleic acids sequences for controlling gene expression with the use of a small interfering RNA (siRNA, formerly known as double stranded RNA or dsRNA) approach. It has been described in the art (WO 99/32169) that providing siRNA to a target cell can result in the down regulation of the translation/ expression of any desired RNA sequence that may be present in said cell. As such, the nucleic acids of the present invention can be used as antagonistic or agonistic compounds, and may be employed to regulate the effects of the polypeptides of the present invention on the modulation of bone homeostasis and in particular the induction of osteoblast differentiation and/or BAP expression and activity, both in vitro and in vivo.

Also, the present invention relates to siRNA for use as a medicament, characterized that said siRNA agonises or antagonises osteoblast and/or osteoclast activity and in particular agonises or antagonises the induction of osteoblast differentiation by said polynucleotide sequences.

Accordingly, the present invention relates to a cell, in which the polynucleotide sequences comprising the nucleic acids sequences as described herein have been introduced. It will be understood that said cell could be used as a medicament, in that said cell could be introduced in a patient suffering from pathologies related to the disturbance of bone homeostasis. Repopulating with said cells will be beneficial to the patient.

Therapy and Diagnosis

As described in the introduction, a large number of diseases are caused by a disturbance of the fine-tuned balance between bone resorption and bone build-up, i.e., bone homeostasis. The single most important bone disease resulting from disturbed bone homeostasis is osteoporosis, but a number of other diseases resulting from a disturbed bone homeostasis represent a large number of patients, such as, for example, hypercalcemia of malignancy, Paget's disease, inflammatory bone diseases like rheumatoid arthritis and periodontal disease, focal osteogenesis occurring during skeletal and other bone metastases, Crouzon's syndrome, rickets, opsismodysplasia, pycnodysostosis/Toulouse-Lautrec disease, osteogenesis imperfecta (Karsenty, 1999; Teitelbaum, 2000; Rodan and Martin, 2000; Goltzman, 2001; Karsenty, 2001). Hence, increases or decreases of osteoclast activity or increases or decreases of osteoblast proliferation and differentiation result in a variety of diseases.

Therefore, it is contemplated that return to bone homeostasis is beneficial to a patient, in the case of bone homeostasis being disturbed. Similarly, it is anticipated that in certain cases, such as, for example, bone fracture or osteoporosis, induction of osteoblast differentiation will be beneficial to a patient.

Hence, the present invention relates to a nucleic acid, a polypeptide or antibody according to the present invention, for use as a medicament.

In a preferred embodiment, the present invention relates to the use of a nucleic acid, a polypeptide or antibody according to the present invention for the preparation of a medicament for preventing, treating and/or alleviating diseases involving the disturbance of bone homeostasis, and in particular the absence or decreased induction of osteoblast differentiation.

In a preferred embodiment, the present invention relates to the use of a nucleic acid identifiable by a method as described herein for the preparation of a medicament for preventing, treating and/or alleviating diseases involving the disturbance of bone homeostasis, and in particular the absence or decreased induction of osteoblast differentiation.

In a preferred embodiment, the present invention relates to a pharmaceutical composition comprising a substantially purified nucleic acid, polypeptide or antibody according to the present invention for preventing, treating and/or alleviating diseases involving the disturbance of bone homeostasis, and in particular the absence or decreased induction of osteoblast differentiation, and possibly in conjunction with a suitable carrier.

Suitable carriers for adding to the nucleic acids, polypeptides or antibodies of the present invention are well known in the art.

In a preferred embodiment, the present invention provides polypeptides according to the present invention, including protein fusions, or fragments thereof, for regulating bone homeostasis in a desired target cell, in vitro or in vivo.

In another embodiment, the present invention contemplates a method for preventing, treating and/or alleviating diseases or disorders involving the disturbance of bone homeostasis comprising the use of a molecule which allows to interfere with the expression of a polynucleotide or a polypeptide as described herein, in a patient.

In another embodiment, the present invention contemplates a method for regulating bone homeostasis comprising:

(a) introducing a sample nucleic acid or an expression vector comprising a sample nucleic acid according to the present invention in a desired host cell, in vitro or in vivo,

(b) expressing said nucleic acid, and,

(c) regulating bone homeostasis by the products expressed by said nucleic acid or the product of said expression vector.

In a preferred embodiment, the present invention further contemplates a method for inducing osteoblast differentiation comprising:

(a) introducing a sample nucleic acid or an expression vector comprising a sample nucleic acid according to the present invention in a desired host cell, in vitro or in vivo,

(b) expressing said nucleic acid, and,

(c) stimulating the differentiation of the host cells into osteoblasts.

In a preferred embodiment, the invention provides polypeptides, including protein fusions, or fragments thereof, for regulating bone homeostasis, and in particular osteoblast differentiation in a desired target cell, in vitro or in vivo. For example, inhibition of osteoblast and/or osteoclast activity or the induction of osteoblast differentiation may occur as a direct result of administering polypeptides to mammalian, preferably human, cells. Delivering compositions containing the polypeptide of the invention to target cells, may occur via association via heterologous polypeptides, heterologous nucleic acids, toxins, or pro-drugs via hydrophobic, hydrophilic, ionic and/or covalent interactions.

Delivery of polypeptides or polynucleotides to the nucleus of a cell nucleus can be accomplished by fusing the polypeptides or polynucleotides to a short component (16 or 7 amino acids) of the Antennapedia protein from drosophila (Penetratin®, Cyclacel). The Antennapedia peptide will direct the polypeptide or polynucleotide to the nucleus of a cell. Also, the protein transduction domain (PTD) from the human immunodeficiency TAT protein and herpes simplex virus type 1 (HSV-1) virion protein VP22 will both guide polypeptides or polynucleotides into a cell when fused to the polypeptides or polynucleotides (Cao G et. al. J Neurosci Jul. 1, 2002; 22(13):5423-31, H Nagahara, et. al Nature Medicine 4, 1449-1452 (1998), Morris et al. Biot (2001) vol 19 1173-1176).

A further aspect of the invention provides a method for treating conditions characterized by decreases in bone mass, which method comprises administering to a subject in need thereof an effective bone mass-increasing amount of a biologically active peptide, wherein said peptide comprises an amino acid sequence at least 90% identical to a member selected from the group consisting essentially of SEQ ID NO: 2, 4, 6, 8, or 10.

A great variety of expression systems can be used. Such systems include, among others, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses, adeno-associated virus, and retroviruses, and vectors derived from combinations thereof, such as those derived from plasmid and bacteriophage genetic elements, such as cosmids and phagemids. The expression systems may contain control regions that regulate as well as engender expression. Generally, any system or vector suitable to maintain, propagate or express polynucleotides to produce a polypeptide in a host may be used. The appropriate nucleotide sequence may be inserted into an expression system by any of a variety of well-known and routine techniques, such as, for example, those set forth in Sambrook et al., Molecular Cloning: A Laboratory Manual (supra).

In a particular aspect of the invention, a fusion of peptide guiding sequences, as described above, with amino acid sequences selected from the group consisting of SEQ ID NO: 2, 4, 6, 8, or 10 can be used.

This dosage may be delivered in a conventional pharmaceutical composition by a single administration, by multiple applications, or via controlled release, as needed to achieve the most effective results, preferably one or more times daily by injection.

The selection of the exact dose and composition and the most appropriate delivery regimen will be influenced by, inter alia, the pharmacological properties of the selected compounds of SEQ ID NO: 2, 4, 6, 8, or 10 or derivatives thereof, the nature and severity of the condition being treated, and the physical condition and mental acuity of the recipient. Representative preferred delivery regimens include, without limitation, oral, parenteral (including subcutaneous, transcutaneous, intramuscular and intravenous), rectal, buccal (including sublingual), transdermal, and intranasal insufflation.

Pharmaceutically acceptable salts retain the desired biological activity of the compounds of SEQ ID NO: 2, 4, 6, 8, or 10 or derivatives thereof without toxic side effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, pamoic acid, alginic acid, polyglutamic acid, naphthalenesulfonic acids, naphthalene disulfonic acids, polygalacturonic acid and the like; (b) base addition salts formed with polyvalent metal cations such as zinc, calcium, bismuth, barium, magnesium, aluminum, copper, cobalt, nickel, cadmium, and the like; or with an organic cation formed from N,N′-dibenzylethylenediamine or ethylenediamine; or (c) combinations of (a) and (b), e.g., a zinc tannate salt and the like. A further aspect of the present invention relates to pharmaceutical compositions comprising as an active ingredient compounds of SEQ ID NO: 2, 4, 6, 8, or 10 or derivatives thereof of the present invention, or pharmaceutically acceptable salt thereof, in admixture with a pharmaceutically acceptable, non-toxic carrier. As mentioned above, such compositions may be prepared for parenteral (subcutaneous, transcutaneous, intramuscular or intravenous) administration, particularly in the form of liquid solutions or suspensions; for oral or buccal administration, particularly in the form of tablets or capsules; for rectal, transdermal administration; and for intranasal administration, particularly in the form of powders, nasal drops or aerosols. The compositions may conveniently be administered in unit dosage form and may be prepared by any of the methods well-known in the pharmaceutical art, for example as described in Remington's Pharmaceutical Sciences, 17th ed., Mack Publishing Company, Easton, Pa., (1985). Formulations for parenteral administration may contain as excipients sterile water or saline, alkylene glycols such as propylene glycol, polyalkylene glycols such as polyethylene glycol, oils of vegetable origin, hydrogenated naphthalenes and the like. For oral administration, the formulation can be enhanced by the addition of bile salts or acylcarnitines. Formulations for nasal administration may be solid and may contain excipients, for example, lactose or dextran, or may be aqueous or oily solutions for use in the form of nasal drops or metered spray. For buccal administration typical excipients include sugars, calcium stearate, magnesium stearate, pregelatinated starch, and the like.

Delivery of the compounds of the present invention to the subject over prolonged periods of time, for example, for periods of one week to one year, may be accomplished by a single administration of a controlled release system containing sufficient active ingredient for the desired release period. Various controlled release systems, such as monolithic or reservoir-type microcapsules, depot implants, osmotic pumps, vesicles, micelles, liposomes, transdermal patches, iontophoretic devices and alternative injectable dosage forms may be utilized for this purpose. Localization at the site to which delivery of the active ingredient is desired is an additional feature of some controlled release devices, which may prove beneficial in the treatment of certain disorders. One form of controlled release formulation contains the polypeptide or its salt dispersed or encapsulated in a slowly degrading, non-toxic, non-antigenic polymer such as copoly(lactic/glycolic)acid, as described in U.S. Pat. No. 4,675,189. The compounds or, preferably, their relatively insoluble salts, may also be formulated in cholesterol or other lipid matrix pellets, or silastomer matrix implants. Additional slow release, depot implant or injectable formulations will be apparent to the skilled artisan. See, for example, Sustained and Controlled Release Drug Delivery Systems, J. R. Robinson ed., Marcel Dekker, Inc., New York, 1978, and R. W. Baker, Controlled Release of Biologically Active Agents, John Wiley & Sons, New York, 1987.

The compounds of SEQ ID NO: 2, 4, 6, 8, or 10 and derivatives thereof may be administered in combination with other agents useful in treating a given clinical condition. When treating osteoporosis and other bone-related disorders for example, the compounds of SEQ ID NO: 2, 4, 6, 8, or 10 and derivatives thereof may be administered in conjunction with a dietary calcium supplement or with a vitamin D analog (see U.S. Pat. No. 4,698,328). Alternatively, the compounds of SEQ ID NO: 2, 4, 6, 8, or 10 and derivatives thereof may be administered, preferably using a cyclic therapeutic regimen, in combination with bisphosphonates, as described for example in U.S. Pat. No. 4,761,406, or in combination with one or more bone therapeutic agents such as, without limitation, calcitonin and estrogen.

Compounds of SEQ ID NO: 2, 4, 6, 8, or 10 or derivatives thereof of this invention are useful for the prevention and treatment of a variety of mammalian conditions manifested by loss of bone mass. In particular, the compounds of this invention are indicated for the prophylaxis and therapeutic treatment of osteoporosis and osteopenia in humans. Furthermore, the compounds of this invention are indicated for the prophylaxis and therapeutic treatment of other bone diseases. Finally, the compounds of this invention are indicated for use as agonists for fracture repair and as antagonists for hypercalcemia.

In one preferred embodiment the present invention provides a gene therapy method for treating, alleviating or preventing disorders and diseases involving pathological disturbance of bone homeostasis. The gene therapy methods relate to the introduction of nucleic acid sequences into an animal to achieve expression of a polypeptide of the present invention. This method requires a nucleic acid, which codes for a polypeptide of the invention that is operatively linked to a promoter or any other genetic element necessary for the expression of the polypeptide in the target tissue. Such gene therapy and delivery techniques are known in the art, see, for example, EP0707071.

In one embodiment, the nucleic acids of the invention is delivered as a naked polynucleotide. The term naked nucleic acid refers to sequences that are free from any delivery vehicle that acts to assist, promote or facilitate entry into a cell, including viral sequences, viral particles, liposome formulations, lipofectin or precipitating agents and the like. The naked nucleic acids can be delivered by any method known in the art, including, but not limited to, direct needle injection at the delivery site, intravenous injection, topical administration, catheter infusion, and so-called “gene guns”.

In another embodiment nucleic acids of the invention may also be linked to a selected tissue-specific promoter and/or enhancer and the resultant hybrid gene introduced, by standard methods (e.g., as described by Leder et al., U.S. Pat. No. 4,736,866, herein incorporated by reference), into an animal embryo at an early developmental stage (e.g., the fertilized oocyte stage), to produce a transgenic animal which expresses elevated levels of compounds of SEQ ID NO: 2, 4, 6, 8, or 10 or derivatives thereof in selected tissues (e.g., the osteocalcin promoter for bone). Such promoters are used to direct tissue-specific expression of compounds of SEQ ID NO: 2, 4, 6, 8, or 10 or derivatives thereof in the transgenic animal. In another embodiment, the nucleic acids of the present invention may be delivered with delivery vehicles such as viral sequences, viral particles, liposome formulations, lipofectin or precipitating agents and the like. Viral vectors that can be used for gene therapy applications include, but are not limited to, a herpes virus vector, a baculovirus vector, a lentivirus vector, a retrovirus vector, an alphavirus vector, an adeno-associated virus vector or an adenoviral vector or any combination thereof. In a preferred embodiment, viral vectors used are replication deficient, for example such as described for adenoviral vectors in WO99/64582.

Delivery of the nucleic acids into a subject may be either direct, in which case the subject is directly exposed to the nucleic acid or nucleic acid-carrying vectors, or indirect, in which case cells are first transformed with the nucleic acids in vitro, and then transplanted into the subject. These two approaches are known, respectively, as in vivo or ex vivogene therapy and are well described. In addition, the polypeptides according to the invention can be used to produce a biopharmaceutical. The term “biopharmaceutical” relates to a recombinantly or synthetically produced polypeptide or protein. Means to recombinantly or synthetically produce polypeptides or proteins are well known in art, such as for example described in Sambrook et al. (1987). Said biopharmaceutical can be applied in vivo, such as for example intravenously or subcutaneously. Alternatively, said biopharmaceutical can be applied in vivo, such as for example by isolating cells of patient, after which the cells are treated with said biopharmaceutical. Subsequently, said treated cells are re-introduced into said patient.

In a more preferred embodiment, the present invention provides a gene therapy method for treating, alleviating or preventing disorders and diseases involving pathological disturbance of bone homeostasis comprising the use of the vectors according to the present invention.

Cells into which nucleic acids or polypeptides of the present invention can be introduced, for example for therapeutic purposes, encompass any desired available cell type, including but not limited to mesenchymal cells, progenitors of mesenchymal cells, and various stem cells, in particular mesenchymal stem cells.

In a preferred embodiment, the invention provides a method for treating, alleviating or preventing disorders involving pathological disturbance of bone homeostasis comprising the use of a molecule, which allows to interfere with the expression of a polynucleotide and/or expression and/or functional activity of a polypeptide of the present invention in a patient in need of such a treatment.

Accordingly, the present invention relates to a cell, in which the polynucleotide sequences comprising the nucleic acids sequences as described herein have been introduced. It will be understood that said cell could be used as a medicament, in that said cell could be introduced in a patient suffering from pathologies related to the disturbance of bone homeostasis. Repopulating with said cells will be beneficial to the patient.

It will be understood that the present invention relates to a transgenic non-human animal comprising one or more copies of a nucleic acid of invention stably integrated into the genome of said animal, or an animal comprising regulatory elements that modulate the expression of a nucleic acid of the present invention.

A gene can be knocked-out by various means, therefore a preferred embodiment of the present invention pertains to a knock-out non-human animal comprising a deletion of one or two alleles encoding a nucleic acid of the present invention, or the deletion of one or more exons of said nucleic acid, or an animal comprising a targeted mutation in the genomic region, including regulatory sequences, comprising any of the nucleic acid sequences of the present invention. In general, a knock-out will result in the ablation of the function of the particular gene.

An even more preferred embodiment of the present invention pertains to the use of a transgenic or knock-out non-human animal according to the present invention as a model system for bone homeostasis, in particular osteoblast differentiation, and/or disease affecting bone homeostasis.

In another embodiment, the present invention provides antibody-based therapies for regulating bone homeostasis in a desired target cell, in vitro, in vivo or ex vivo.

Antibody-based therapies involve administering of anti-polypeptide or anti-polynucleotide antibodies to a mammalian, preferably human, cell. Methods for producing anti-polypeptide and anti-polynucleotide antibodies are known in the art. Such antibodies may be provided in pharmaceutically acceptable compositions as known in the art.

The invention provides a method for producing a substrate with a matrix grown thereon, which can be used preferably for the provision of load-bearing implant, including joint prostheses, such as artificial hip joints, knee joints and finger joints, and maxillofacial implants, such as dental implants. It can also be used for special surgery devices, such as spacers, or bone fillers, e.g., for use in augmentation, obliteration or reconstitution of bone defects and damaged or lost bone. Bone formation can be optimized by variation in mineralization, both by inductive and by conductive processes. A combination of the provision of a load-bearing implant (preferably coated with a matrix as described above) with a bone filler comprising a matrix as described, constitutes an advantageous method according to the present invention.

The method of the invention is also very suitable in relation to revision surgery, i.e., when previous surgical devices have to be replaced.

Undifferentiated cells are pluripotent cells which are in an early stage of specialization, i.e., which do not yet have their final function and can be induced to form almost any given cell type. In particular, these are cells which have not yet differentiated to e.g., osteoblasts or osteoclasts. Such cells are especially blood cells and cells present in bone marrow, as well as cells derived from adipose tissue. In addition, cells which still can be differentiated into MPC are contemplated in the present invention, such as, for example, totipotent stem cells such as embryonic stem cells. Especially suitable undifferentiated cells are bone marrow cells, including haematopoietic cells and in particular stromal cells. The marrow cells, and especially the stromal cells are found to be very effective in the bone producing process when taken from their original environment.

The undifferentiated cells can be directly applied on the substrate or they can advantageously be multiplied in the absence of the substrate before being applied on the substrate. In the latter mode, the cells are still largely undifferentiated after multiplication and, for the purpose of the invention, they are still referred to as undifferentiated. Subsequently, the cells are allowed to differentiate. Differentiation can be induced or enhanced by the presence of suitable inductors, such as glucocorticoids, e.g., dexamethasone. Especially suitable inductors of differentiation are the polynucleotides and polypeptides of the present invention.

The use of undifferentiated cells provides several advantages. Firstly, their lower differentiation implies a higher proliferation rate and allows the eventual functionality to be better directed and controlled. Moreover, culturing these cells not only produces the required bone matrix containing organic and inorganic components, but also results in the presence, in the culture medium and in the matrix, of several factors which are essential for growth of the tissue and for adaptation to existing living tissue. Also, the culture medium can be a source of active factors such as growth factors, to be used in connection with the implanting process. Furthermore, such undifferentiated cells are often available in larger quantities and more conveniently than e.g., mature bone cells, and exhibit a lower morbidity during recovery. Matrices as thick as 100 μm can be produced as a result of the use of undifferentiated cells.

The cells to be used can be allogeneous cells, but it will often be preferred to use cells originating from the same subject for which the implant is intended, i.e., autologous cells.

The substrate on which the undifferentiated cells can be applied and cultured can be a metal, such as titanium, cobalt/chromium alloy or stainless steel, a bioactive surface such as a calcium phosphate, polymer surfaces such as polyethylene, and the like. Although less preferred, siliceous material such as glass ceramics, can also be used as a substrate. Most preferred are metals, such as titanium, and calcium phosphates, even though calcium phosphate is not an indispensable component of the substrate. The substrate may be porous or non-porous.

The cells can be applied at a rate of e.g., 10³-10⁶ per cm², in particular 10⁴-2×10⁵ cells per cm².

The culture medium to be used in the method according to the invention can be a commonly known culture medium such as MEM (minimum essential medium). Advantageously, the medium can be a conditioned medium. In this context, a conditioned medium is understood to be a medium wherein similar cells have previously been incubated, causing the medium to contain factors such as polypeptides, secreted by the cells which are important for cell growth and cell differentiation.

Preferably, a polynucleotide of the present invention is introduced into the cells, under conditions such that said polynucleotide is expressed. Also, a polypeptide of the present invention is applied to the cells.

The cells are cultured for a time to produce a sufficient matrix layer, e.g., a matrix layer having a thickness of at least 0.5 μm, in particular from 1 up to 100 μm, more in particular of 10-50 μm. The cells may be contacted with the culture medium for e.g., 2-15 weeks, in particular 4-10 weeks.

The production of the matrix, when applied on a substrate, results in a continuous or quasi-continuous coating covering the substrate for at least 50%, in particular at least 80% of its surface area.

Hence, the invention also pertains to the use of a nucleic acid or polypeptide as described herein for the induction of bone tissue production, in particular to differentiate undifferentiated cells and form osteoblasts and to produce continuous bone matrix.

In a preferred embodiment, the present invention thus provides a method for in vitro production of bone tissue, comprising the steps of:

(a) applying undifferentiated mammalian cells on a substrate to form a cellular substrate,

(b) introducing a polynucleotide of the present invention into said cells, or contacting the cellular substrate with the polypeptide according to the present invention for a time sufficient to differentiate the undifferentiated cells into osteoblasts, thereby producing a continuous bone matrix.

In a preferred embodiment, the bone matrix comprises a thickness of at least about 0.5 μm on the surface of the substrate.

In another embodiment, the present invention relates to a method for producing an implant, said method comprising:

(a) isolating a person's mesenchymal pluripotent cells (MPCs) from, for example, bone marrow, fat tissue or other autologous sources containing MPCs

(b) inducing ex vivo differentiation of said MPCs to osteoblasts by introducing polynucleotide sequences comprising a nucleic acid according to the present invention into said MPCs, or by applying a polypeptide according to the present invention to said MPCs,

(c) possibly mixing the osteoblasts of step (c) with a matrix or matrix-forming material, and,

(d) covering synthetic grafts with the osteoblasts of step (b) or with the mix of the osteoblast with the matrix or matrix-forming material of step (c) to produce an implant.

In another embodiment, the present invention relates to a method for producing a implant, said method comprising:

(a) isolating a person's mesenchymal pluripotent cells (MPCs) from bone marrow, fat tissue or other autologous sources containing MPCs,

(b) inducing ex vivo differentiation of said MPCs to osteoblasts by introducing polynucleotide sequences comprising a nucleic acid according to the present invention into said MPCs, or by applying a polypeptide according to the present invention to said MPCs, whereby an implant is produced, and,

(c) possibly mixing the osteoblasts of step (b).

In yet another embodiment the osteoblasts of step (b) are combined with a matrix or matrix-forming material, whereby an implant is produced.

The ex vivo differentiated osteoblasts, possibly mixed with a matrix or matrix-forming material, produced by the methods of the present invention can be used for direct engraftment of intravenous, subcutaneous, by bone marrow transplantation or any other route that can possibly be used to successfully transplant the ex vivo differentiated MPCs.

In another embodiment, the present invention relates to the use of a polypeptide according to the present invention for the differentiation of an undifferentiated cell to an osteoblast or osteoclast.

In another embodiment, the present invention relates to the use of a differentiated cell obtainable by a method described herein for the manufacture of an implant.

In a preferred embodiment, the present invention relates to a composition for the treatment of defects in bones comprising a matrix or matrix-forming material used to fill a defect in bone and a polypeptide according to the present invention, at a concentration sufficient to induce osteoblast differentiation.

In another preferred embodiment, the present invention relates to a composition for the treatment of defects in bones comprising a matrix or matrix-forming material used to fill a defect in bone and an effective amount of a transfectable vector according to the present invention.

In a preferred embodiment, the present invention provides the nucleic acids, polypeptides or antibodies of the present invention for use as a medicament (both for treatment as for diagnosis of diseases). Said treatment according to the present invention refers to preventing, treating and/or alleviating diseases or disorders involving pathological disturbance of bone homeostasis or the absence or decreased induction of osteoblast differentiation as defined above and below.

Consequently, the present invention relates to the use of a nucleic acid, polypeptide or antibody as described herein for the preparation of a diagnostic kit for detecting disregulated bone homeostasis, and in particular absence or decreased osteoblast differentiation.

In a further embodiment, the invention provides a method for diagnosing a pathological condition or a susceptibility to a pathological condition relating to the disturbance of bone homeostasis, including for example abnormal osteoblast differentiation, in a subject comprising the steps of:

(a) determining the presence or absence of a mutation in the nucleic acid according to the present invention, including mutations in the genomic and regulatory sequences of said nucleic acid, in a biological sample,

(b) diagnosing a pathological condition or a susceptibility to a pathological condition based on the presence or absence of said mutation.

In a further embodiment, the invention provides a method for diagnosing a pathological condition relating to abnormal osteoblast differentiation or a susceptibility to said condition in a subject comprising:

a) obtaining a sample of said subject's mRNA corresponding to a nucleic acid of the present invention or a sample of said subject's genomic DNA corresponding to a genomic sequence of a nucleic acid of the present invention;

b) determining the nucleic acid sequence of said subject's mRNA or genomic DNA;

c) comparing the nucleic acid sequence of said subject's mRNA or genomic DNA with a nucleic acid of the present invention or with a genomic sequence encoding a nucleic acid of the present invention obtained from a database; and

d) identifying any difference(s) between the nucleic acid sequence of said subject's mRNA or genomic DNA and said nucleic acid of the present invention or said genomic sequence encoding a nucleic acid of the present invention obtained from a database.

It is understood that a subject's mRNA “corresponds to” a nucleic acid of the present invention when the subject's mRNA is transcribed from the same gene as the nucleic acid of the present invention. Furthermore, it is understood that a subject's genomic DNA “corresponds to” a genomic sequence encoding a nucleic acid of the present invention when the subject's genomic DNA encodes for the same gene as the nucleic acid of the present invention.

It is well understood in the art that databases such as GenBank can be searched to identify genomic sequences that contain regions of identity (exons) to a nucleic acid. Such genomic sequences are thus said to encode for the nucleic acid.

In an even further embodiment, the present invention pertains to a method for diagnosing a pathological condition or a susceptibility to a pathological condition relating to the disturbance of bone homeostasis, including for example abnormal osteoblast differentiation, in a subject comprising the steps of:

(a) determining the presence or amount of the nucleic acid to the present invention or expression of the polypeptide to the present invention in a biological sample, and,

(b) diagnosing a pathological condition or a susceptibility to a pathological condition relating to the disturbance of bone homeostasis, including for example abnormal osteoblast differentiation, based on the presence or amount of said nucleic acid or expression of said polypeptide.

In another embodiment, the invention provides a method for diagnosing a pathological condition and/or a susceptibility to pathological condition relating to the disturbance of bone homeostasis in a subject comprising the steps of:

(a) determining the presence of a chromosomal translocation of the gene encoding for the nucleic acid of the present invention, and,

(b) diagnosing the pathological condition and/or a susceptibility to pathological condition relating to the disturbance of bone homeostasis, based on the presence of a chromosomal translocation of said gene.

The diagnosis as described in the present invention may be preferably achieved by means of detection using probes, primers or antibodies of the invention.

In a preferred embodiment, the invention provides a kit for the diagnosis of detecting disregulated bone homeostasis, including for example abnormal osteoblast differentiation, in a patient comprising a nucleic acid of the invention, a probe or primer according of the invention, a polypeptide of the invention, or an antibody of the invention, possibly in conjunction with suitable buffers, means for detection or detection format parts (such as, for example, solid carriers, e.g., membranes). Suitable formats and technologies for designing diagnostic kits on the basis of the above are well known in the art. Preferred formats include any type of microarray format known in the art.

Drug Screens

The invention provides methods for identifying compounds or agents that can be used to treat disorders characterized by (or associated with) the disturbance of bone homeostasis, in particular absence or decreased osteoblast differentiation and/or increased osteoclast proliferation, differentiation and activity. These methods are also referred to herein as “drug screening assays” or “bioassays” and typically include the step of screening a candidate/test compound or agent for the ability to interact with (e.g., bind to) a protein of the present invention, in particular represented by SEQ ID NO: 2, 4, 6, 8, or 10, or any derivative, homologue, immunologically active or functional fragment thereof, to modulate the interaction of the protein of the present invention and a target molecule, and/or to modulate the expression of the nucleic acids of the present invention and/or activity of the proteins of the present invention. Candidate/test compounds or agents which have one or more of these abilities can be used as drugs to treat disorders characterized by pathological disturbance of bone homeostasis, disregulated expression of the nucleic acids of the present invention and/or disregulated functional activity of the proteins of the present invention. Candidate/test compounds such as antibodies, small molecules, e.g., small organic molecules and peptides, and other drug candidates can be obtained, for example, from combinatorial and natural product libraries.

The screening for therapeutic compounds may be any of a variety of drug screening techniques known in the art.

Thus, the present invention relates to the use of the nucleic acids, the polypeptides or antibodies as described herein for drug or test compound screens directed to identify drugs, test compounds or antibodies that interfere with bone homeostasis, and in particular osteoblast differentiation.

In one embodiment, the invention provides a drug screening assay for screening candidate/test compounds which interact with (e.g., bind to) the polypeptides or proteins of the present invention, or any variant or a derivative thereof, or an immunologically active and/or functional fragment thereof. Typically, the assays are cell-free assays which include the steps of combining the polypeptides or proteins of the present invention, variants or derivatives thereof, its catalytic or immunogenic active and/or functional fragments thereof, and a candidate/test compound, e.g., under conditions which allow for interaction of (e.g., binding of) the candidate/test compound to the polypeptide or protein of the present invention, or any variant or a derivative thereof, or an immunologically active and/or functional fragment thereof, to form a complex, and detecting the formation of a complex, in which the ability of the candidate compound to interact with (e.g., bind to) the polypeptide or proteins of the present invention, or any variant or a derivative thereof, or an immunologically active and/or functional fragment thereof is indicated by the presence of the candidate/test compound in the complex. Formation of complexes between the protein of the present invention and the candidate compound can be quantitated, for example, using standard immunoassays.

The proteins of the present invention, its catalytic or immunogenic fragments or oligopeptides thereof employed in such a test may be free in solution, affixed to a solid support, borne on a cell surface, or located intracellularly.

In another embodiment, the invention provides screening assays to identify candidate/test compounds which modulate (e.g., stimulate or inhibit) the interaction (and most likely the functional activity of the proteins of the present invention as well) between a protein of the present invention and a molecule (target molecule) with which the protein of the present invention normally interacts, or antibodies which specifically recognize the protein of the present invention. Examples of such target molecules include proteins in the same signalling path as the protein of the present invention, e.g., proteins which may function upstream (including both stimulators and inhibitors of activity) or downstream of the signalling pathways of the proteins of the present invention.

Typically, the assays are cell-free assays which include the steps of combining a polypeptide or protein of the present invention, a protein target molecule and a candidate/test compound, e.g., under conditions wherein but for the presence of the candidate compound, the polypeptide or protein of the present invention interacts with (e.g., binds to) the target molecule, and detecting the formation of a complex which includes the polypeptide or protein of the present invention, or detecting the interaction/reaction of the polypeptide or protein of the present invention, and the target molecule. Examples of such target molecules include proteins in the same signalling path as the protein of the present invention, e.g., proteins which may function upstream (including both stimulators and inhibitors of activity) or downstream of the signalling pathways of the proteins of the present invention, or ligands or known receptors or antibodies specifically recognizing the polypeptide or protein of the invention.

It is understood that also variants, or a derivative or an catalytic, or immunologically active and/or functional fragment of the polypeptide and proteins from the present invention can be used in the drug screening assay as described above.

In another embodiment, the present invention provides a drug screening assay for the identification of test compounds which synergizes the activity, preferably the modulation of bone homeostasis, and even more preferably the induction of osteoblast differentiation, of a nucleic acid according to the present invention or of a polypeptide according to the present invention, said assay comprising:

(a) providing a host cell,

(b) combining a test compound and a nucleic acid according to the present invention or of a polypeptide according to the present invention, under conditions wherein said test compound interacts with said a nucleic acid according to the present invention or of a polypeptide according to the present invention,

(c) applying the product of step (b) to said host cell, and

(d) determining the induction of osteoblast differentiation of said host cell, and thereby identifying the synergistic activity of said test compound.

In another embodiment, the present invention provides for a drug screening assay for the identification of test compounds which modulate the interaction between a protein that induces osteoblast differentiation and a target molecule, comprising:

(a) combining (i) a test compound, (ii) an agent consisting of a polypeptide according to the present invention, or a variant or a derivative thereof, or an immunologically active and/or functional fragment thereof, and (iii) a target molecule to which said agent interacts in the absence of a test compound, under conditions that allow the interaction thereof, and

(b) detecting the formation of a complex that includes either said agent and the target molecule, or said agent and the test compound.

In another embodiment, the present invention provides a drug screening assay for the identification of test compounds which modulate, and preferably agonize or antagonize, osteoblast differentiation, said assay comprising:

(a) combining a test compound and a protein target molecule on a host cell, under conditions wherein said test compound interacts with said protein target molecule,

(b) determining the induction of osteoblast differentiation of said host cell,

(c) combining the polypeptide according to the present invention, or a variant or a derivative thereof, or an immunologically active and/or functional fragment thereof, and a protein target molecule on a host cell, under conditions wherein said polypeptide, or a variant or a derivative thereof, or an immunologically active and/or functional fragment thereof, interacts with said protein target molecule, and,

(d) determining the induction of osteoblast differentiation of said host cell,

(e) determining the difference in induction of osteoblast differentiation of step (b) compared to step (d), and thereby identifying the test compound which modulates, and preferably antagonizes or agonizes, osteoblast differentiation.

In another preferred embodiment, the present invention provides for a drug screening method for the identification of test compounds that modulate the expression of a osteoblast differentiation gene comprising a nucleic acid of the present invention, said assay comprising:

(a) providing a host cell transfected with an expression vector comprising the nucleic acid according to the present invention,

(b) introducing a test compound into said host cell, and

(c) measuring the expression levels of polypeptide encoded by said nucleic acid.

In another preferred embodiment, the present invention provides for a competitive drug screening assay comprising:

(a) providing an antibody according to the present invention and combining said antibody with a test compound for binding to an agent consisting of a polypeptide according to the present invention, or a variant or a derivative thereof, or an immunologically active and/or functional fragment thereof, wherein said antibody and said test compound compete for binding to said agent and,

(b) determining the relative binding affinity of said antibody and said test compound for said agent.

In another preferred embodiment, the present invention provides for a drug screening assay for identifying compounds that modulate the interaction between binding partners in a complex, in which at least one of said binding partners is an agent consisting of a polypeptide according to the present invention, or a variant or a derivative thereof, or an immunologically active and/or functional fragment thereof, said method comprising:

(a) contacting a test compound with the complex, for a time sufficient to modulate the interaction in the complex;

(b) monitoring said complex for changes in complex interaction;

(c) identifying test compounds that alter interactions in said complex.

In case that the nucleotide sequences of the present invention would only be a part of a coding region, the nucleotide sequence may be extended to the complete coding sequence, by employing various methods known in the art. Similarly, the nucleotide sequences of the present invention may be used in various methods known in the art to identify the gene corresponding to said nucleotide sequences, including regulatory elements such as promoters, enhancers, silencers, transcription start sites, CpG-islands, internal ribosomal entry sites, and the like. As such, the invention provides means and methods to regulate the expression of said nucleic acids by providing to a subject or host cell molecules that can positively or negatively influence said regulatory elements of said sequences identified in the present invention, e.g., by drugs, genetic modifier elements, chimeric zinc finger-containing proteins or any other method known in the art.

In particular, the present invention provides a drug screening method for the identification of test compounds which modulate the expression of a gene or genes according to the present invention, said assay comprising:

(a) providing a host cell comprising a gene corresponding to the nucleic acid according to the invention,

(b) introducing a test compound into said host cell, under conditions whereby said test compound modulates the expression of said gene, and,

(c) determining the expression of said gene, thereby identifying the test compound which modulates the expression of said gene

Detection of complex formation can include direct quantitation of the complex by, for example, measuring inductive effects of the protein of the present invention. A statistically significant change, such as a decrease, in the interaction of the protein of the present invention and target molecule (e.g., in the formation of a complex between the protein of the present invention and the target molecule) in the presence of a candidate compound (relative to what is detected in the absence of the candidate compound) is indicative of a modulation (e.g., stimulation or inhibition) of the interaction between the protein of the present invention and the target molecule. Modulation of the formation of complexes between the protein of the present invention and the target molecule can be quantitated using, for example, an immunoassay.

Therefore, the present invention contemplates a drug screening assay for identifying compounds that modulate the interaction between binding partners in a complex, in which at least one of said binding partners is the polypeptide according to the present invention, or a variant or a derivative thereof, or an immunologically active and/or functional fragment thereof, and said method comprising:

(a) contacting a test compound with the complex, for a time sufficient to modulate the interaction in the complex; and thereafter

(b) monitoring said complex for changes in interactions, so that if a change in the interaction is detected, a compound that modulates the interaction is identified.

It should be clear that modulators for interaction between binding partners in a complex, when identified by any of the herein described methods, is contemplated in the invention. In particular, the present invention contemplates the product or compound identifiable by any of the herein described methods.

Also, the present invention contemplates a method for the production of a composition comprising the steps of admixing a compound identifiable by the assay as described herein with a pharmaceutically acceptable carrier.

It will be clear that the present invention contemplates a composition comprising the product or compound identifiable by any of the herein described methods.

Moreover, the present invention contemplates the use of the product or compound identifiable by any of the herein described methods as medicament.

To perform the above described drug screening assays, it is feasible to immobilize either the protein of the present invention or its target molecule to facilitate separation of complexes from uncomplexed forms of one or both of the proteins, as well as to accommodate automation of the assay. Interaction (e.g., binding of) of the protein of the present invention to a target molecule, in the presence and absence of a candidate compound, can be accomplished in any vessel suitable for containing the reactants. Examples of such vessels include microtitre plates, test tubes, and microcentrifuge tubes. In one embodiment, a fusion protein can be provided which adds a domain that allows the protein to be bound to a matrix. For example, the protein of the present invention can be “His” tagged, and subsequently adsorbed onto Ni-NTA microtitre plates (Paborsky et al., 1996), or ProtA fusions with the proteins of the present invention can be adsorbed to IgG, which are then combined with the cell lysates (e.g., (35)^{S}-labelled) and the candidate compound, and the mixture incubated under conditions conducive to complex formation (e.g., at physiological conditions for salt and pH). Following incubation, the plates are washed to remove any unbound label, and the matrix is immobilised. The amount of radioactivity can be determined directly, or in the supernatant after dissociation of the complexes. Alternatively, the complexes can be dissociated from the matrix, separated by SDS-PAGE, and the level of the protein binding to the protein of the present invention found in the bead fraction quantitated from the gel using standard electrophoretic techniques.

Other techniques for immobilizing protein on matrices can also be used in the drug screening assays of the invention. For example, either the protein of the present invention or its target molecule can be immobilized utilizing conjugation of biotin and streptavidin. Biotinylated protein molecules of the present invention can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with the protein of the present invention but which do not interfere with binding of the protein to its target molecule can be derivatized to the wells of the plate, and the protein of the present invention can be trapped in the wells by antibody conjugation. As described above, preparations of a protein binding to a protein of the present invention and a candidate compound are incubated in the wells of the plate presenting the protein of the present invention, and the amount of complex trapped in the well can be quantitated. Methods for detecting such complexes, in addition to those described above for the GST-immobilized complexes, include immuno-detection of complexes using antibodies reactive with the target molecule to the proteins of the present invention, or which are reactive with the protein of the present invention and compete with the target molecule; as well as enzyme-linked assays which rely on detecting an enzymatic activity associated with the target molecule.

Another technique for drug screening which provides for high throughput screening of compounds having suitable binding affinity to the protein of the present invention is described in detail in “Determination of Amino Acid Sequence Antigenicity” by Geysen H N, WO Application 84/03564, published on Sep. 13, 1984, and incorporated herein by reference. In summary, large numbers of different small peptide test compounds are synthesized on a solid substrate, such as plastic pins or some other surface. The protein test compounds are reacted with fragments of the protein of the present invention and washed. Bound protein of the present invention is then detected by methods well known in the art. Purified protein of the present invention can also be coated directly onto plates for use in the aforementioned drug screening techniques. Alternatively, non-neutralizing antibodies can be used to capture the peptide and immobilize it on a solid support.

This invention also contemplates competitive drug screening assays in which neutralizing antibodies capable of binding the proteins of the present invention specifically compete with a test compound for binding the protein of the present invention. In this manner, the antibodies can be used to detect the presence of any protein which shares one or more antigenic determinants with the protein of the present invention. In particular, the present invention pertains to a competitive drug screening assay comprising:

(a) competing the antibodies according to the present invention with a test compound for binding to the polypeptides, or a variant or a derivative thereof, or an immunologically active and/or functional fragment thereof, and,

(b) determining the amount of competition of said antibodies compared to said test compound.

In yet another embodiment, the invention provides a method for identifying a compound (e.g., a drug screening assay) capable of use in the treatment of a disorder characterized by (or associated with) pathological disturbance of bone homeostasis, interference with osteoblast differentiation, disregulated expression of the nucleic acids of the present invention or disregulated functional activity of the proteins of the present invention. Similarly, the invention provides a method for identifying a compound (e.g., a drug screening assay) capable of use in the treatment of a disorder characterized by (or associated with) pathological disturbance of bone homeostasis, interference with osteoblast differentiation, disregulated expression of the nucleic acids of the present invention or disregulated functional activity of the proteins of the present invention. The method typically includes the step of assaying the ability of the compound or agent to modulate the expression of the nucleic acid of the present invention or the functional activity of the protein of the present invention thereby identifying a compound for use in the treatment of a disorder characterized by (or associated with) pathological disturbance of bone homeostasis, interference with osteoblast differentiation, disregulated expression of the nucleic acids of the present invention or disregulated functional activity of the proteins of the present invention.

In a preferred embodiment the invention provides a drug compound screening assay to identify polynucleotide or polypeptide test compounds that may be useful in preventing, treating and/or alleviating diseases or disorders involving the abnormal osteoblast differentiation, comprising:

(a) contacting the test compound with a nucleic acid according to the present invention, and

(b) determining whether said compound interacts with the nucleic acid.

Preferably, the invention relates to a drug screening assay for identifying a compound capable of use in the treatment of a disorder characterized by disregulated bone homeostasis, said assay comprising:

(a) providing a cell comprising a nucleic acid according to the present invention, or any part thereof, or a polypeptide according to the present invention, or a variant or a derivative thereof, or an immunologically active and/or functional fragment thereof,

(b) providing the compound to be tested to the cell of step (a), under conditions which allow said compound to interact with said nucleic acid, or any part thereof, or with said polypeptide, or a variant or a derivative thereof, or an immunologically active and/or functional fragment thereof,

(c) assaying the ability of the compound of step (b) to modulate the expression of said nucleic acid, or any part thereof, or the activity or amount of said polypeptide, or a variant or a derivative thereof, or an immunologically active and/or functional fragment thereof, and,

d) identifying the compound for treating a disorder characterized by disregulated bone homeostasis.

The compounds identified according to the herein described drug screening assays can be used to treat, for example, disorders characterized by or associated with disregulated bone homeostasis or disregulated osteoblast differentiation.

In a preferred embodiment the invention provides a drug screening assay for preventing, treating and/or alleviating diseases or disorders involving the disturbance of bone homeostasis, comprising:

(a) contacting the compounds to be screened with a nucleic acid according to the present invention, or a polypeptide according to the present invention, or a variant or a derivative thereof, or an immunologically active and/or functional fragment thereof, and,

(b) determining whether said compound effects an activity, preferably antagonizes said activity, of said nucleic acid or said polypeptide, or a variant or a derivative thereof, or an immunologically active and/or functional fragment thereof.

The present invention also contemplates the use of competitive drug screening assays in which neutralizing antibodies capable of binding polypeptides of the present invention specifically compete with a test compound for binding to the polypeptides or fragments thereof. In this manner, the antibodies are used to detect the presence of any peptide that shares one or more antigenic epitopes with a polypeptide of the invention. In particular, a screening assay for identifying antibodies that modulate the expression or functional activity of the polypeptides of the present invention, or a variant or a derivative thereof, or an immunologically active and/or functional fragment thereof, said method comprising:

(a) providing a cell comprising the polypeptide of the present invention, or a variant or a derivative thereof, or an immunologically active and/or functional fragment thereof,

(b) determining the expression and/or activity of said polypeptide, or a variant or a derivative thereof, or an immunologically active and/or functional fragment thereof,

(c) providing an antibody of the present invention, to the cell of (a), under conditions that said antibody can interact with said polypeptide, or a variant or a derivative thereof, or an immunologically active and/or functional fragment thereof,

(d) determining the modulation of expression and/or activity of said polypeptide, or a variant or a derivative thereof, or an immunologically active and/or functional fragment thereof after said antibody has bound said polypeptides, or a variant or a derivative thereof, or an immunologically active and/or functional fragment thereof.

The assays described herein include but are not limited to an Enzyme Linked Immunosorbent Assays (ELISA) or cell based Enzyme Linked Immunosorbent Assays (CELISA). Such assays allow screening for nucleic acid fragments, polypeptides, and therapeutic compounds using libraries of said compounds or molecules that influence or regulate the expression or function of the polypeptides subject of this invention. The expression of the polypeptides subject of this invention may be influenced, induced or inhibited by a target or test compound. In particular cases, said target or test compound is the expression product of a gene that is introduced into an acceptor cell. Said gene may be derived, for example, from a gene expression library. Said acceptor cells include, but are not limited to, human cells. Said gene expression libraries include, but are not limited to, adenoviral expression libraries.

The disclosure of all patents, publications (including published patent publications), and database accession numbers and depository accession numbers referenced in this specification are specifically incorporated herein by reference in their entirety to the same extent as if each such individual patent, publication, and database accession number, and depository accession number are specifically and individually indicated to be incorporated by reference.

It is to be understood that the following figures and examples are meant to illustrate the embodiments of the present invention and are in no way to be construed as limiting the present invention. The invention may be practiced other than as particularly described and still be within the scope of the accompanying claims.

### SHORT DESCRIPTION OF THE FIGURES

FIG. 1: Schematic diagram of the genetic control of osteoblast differentiation and function.

Several transcription factors and secreted factors are involved in the osteoblast differentiation process. (From Ducy et al., 2000).

FIG. 2. Increasing the transduction efficiency of Ad5 virus by exogenous expression of the Ad5 hCAR receptor.

Cells are first infected with Ad5 or an Ad5 fiber variant expressing the hCAR receptor prior to infection of the cells with the Ad5 viruses of the placental PhenoSelect™ cDNA expression library. This method allows one to infect virtually every single cell line or polyclonal primary cell population.

FIG. 3. Images taken by fluorescence microscopy showing the increase of Ad5 infection in the presence of increasing amounts of hCAR adenoviruses

Optimal MOIs for hCAR and PhenoSelect viruses are determined in a control experiment. MPCs are seeded in wells of a 384 well plate and co-infected using Ad5C01-hCAR at MOIs of 10, 50, and 250. Ad5C01-eGFP (Ad5-GFP) is used at MOIs of 1000, 5000 and 25000. Infection efficiency is analyzed 6 days later using fluorescence microscopy (Zeiss Axiovert 25, 4× objective).

FIG. 4. Flow cytometric analysis depicting the increase of Ad5 infection in the presence of increasing amounts of hCAR adenoviruses

The legend in the upper right corner describes the MOIs used to co-infect MPCs. MOIs for Ad5C01-hCAR are shown on the Y-axis, MOIs for Ad5C01-eGFP are shown on the X-axis

FIG. 5. Principle of the osteoblast differentiation assay.

Mesenchymal stem cells derived from bone marrow are infected with the placenta PhenoSelec™ cDNA library viruses in the presence of Ad5C01- or Ad5C15- or Ad5C20-hCAR virus as outlined in FIG. 2. Six days post infection bone alkaline phosphatase activity is measured following addition of 4-methylumbelliferyl heptaphosphate (MUP) substrate.

FIG. 6. Optimizing seeding densities for AP screen.

MPCs are seeded in a 384 well plate and indicated compounds are added one day later. Six days after adding the compounds (Dex=dexamethasone, bGP=beta-glycerophosphate, BMP2=recombinant human BMP2), alkaline phosphatase activity is measured. Relative fluorescence units are marked on the Y-axis.

FIG. 7. Determining endpoint for AP screen.

MPCs co-infected with Ad5C01-hCAR at an MOI of 100 and a control AD5C01 virus at an MOI of 1000 are treated with recombinant human BMP2 (rhBMP2) and dexamethasone (Dex) one day after seeding. Three, 6, 8 or 10 days later, alkaline phosphatase activity is measured. Relative fluorescence units are marked on the Y-axis.

FIG. 8. Determining length of incubation time for alkaline phosphatase substrate

MPCs are co-infected using Ad5C01-hCAR at an MOI of 100 and AD5C01-PLAP or Ad5C01-BMP2 virus at an MOI of 1000 and treated with dexamethasone (Dex) one day after seeding. Six days later, alkaline phosphatase activity is measured. The incubation time with the MUP-solution is varied from 0, 5, 10, 20 to 60 min. “-” behind every condition marked on the X-axis denotes that no stop-solution is used at the end of the different incubation times. Rather, the samples are read immediately after the end of the different incubation times. Relative fluorescence units are marked on the Y-axis.

FIG. 9. Lay-out of the control plate.

Control plates in a 96 well plate format are prepared containing different control viruses transducing the following transgenes: BMP2, eGFP, PPAR-gamma, empty virus and luciferase.

FIG. 10. Scatter plot for the high-throughput osteoblast differentiation assay.

The figure shows the intra-plate variation in the osteoblast differentiation assay for a typical 384 well plate from the PhenoSelect™ cDNA library. The scattering of data points around the mean for the library samples (each containing a different insert) within the plate is tight. The cut-off value is defined as the mean plus three times the standard deviation. Samples inducing signals above the cut-off value are selected as a hit.

FIG. 11. Hits plate from high-throughput AP screen

Hit H4-2 comprises cDNA sequence (FIG. 13a, SEQ ID NO: 1) that corresponds to human Delta2/Dll4, a presumed Notch ligand.

FIGS. 12a and 12b. Hit validation by RT-PCR

MPCs are infected with Ad5C01-hCAR and Ad5C01 control or hit viruses. Seven days later, RNA is extracted and subjected to PCR using gene specific primers amplifying BAP or PLAP/IAP (degenerate primer set) or beta-actin control reactions. The reaction products are separated on an agarose gel.

FIGS. 13a-d. Nucleotide and deduced amino acid sequences of Hit H4-2 and soluble H4-2.

This cDNA (FIG. 13a) is identical to Homo sapiens hdelta2 for notch ligand delta-2, as described in GenBank record with accession number AB036931. To this gene is also referred to as Homo sapiens delta-like 4 homologue (Drosophila) (DLL4), as described in GenBank record NM_{—}019074. FIG. 13b depicts the deduced amino acid sequence of Dll4. FIG. 13c depicts a nucleic acid encoding for a truncated and soluble form of the Dll4 polypeptide. FIG. 13d depicts the deduced amino acid sequence of soluble Dll4.

FIGS. 14(a-f). Notch intracellular domain-2, -3, and -4 DNA and deduced amino acid sequences.

14a and 14b. The DNA sequence (FIG. 14a; SEQ ID NO: 5) and deduced amino acid sequence (FIG. 14b; SEQ ID NO: 6) of Notch intracellular domain-2 (NICD-2) is depicted.

14c and 14d. The DNA sequence (FIG. 14c; SEQ ID NO: 7) and deduced amino acid sequence (FIG. 14d; SEQ ID NO: 8) of NICD-3 are depicted.

14e and 14f. The DNA sequence (FIG. 14e; SEQ ID NO: 9) and deduced amino acid sequence (FIG. 14f; SEQ ID NO: 10) of NICD-4 are depicted.

FIG. 15. Dll4 polypeptide schematic.

A schematic representation of Dll4 is shown. Dll4 contains seven EGF-like domains flanked on the N-terminus by a Delta/Serrate/Lag-2 domain and transmembrane and intracellular domains on the c-terminus.

FIG. 16. Delta-like ligand/Notch signaling pathway.

H4-2/Dll4 is a ligand for Notch receptors. The Notch receptor is comprised of extracellular and intracellular domains that combine to transduce signals from extracellular ligands to the nucleus.

FIG. 17. Up-regulation of BAP mRNA by H4-2 expression.

mRNA is isolated from cells expressing eGFP, H4-2, and BMP-2 and subjected to PCR using BAP-specific primers.

FIG. 18. Expression of soluble H4-2.

His6-tagged H4-2 is expressed in PER.C6 cells and purified. Western blotting and coomassie staining indicate that the soluble H4-2 protein solution contains only minor impurities.

FIG. 19. Soluble H4-2 induces AP up-regulation.

Microtiter plates are treated with varying concentrations of soluble H4-2 protein prior to seeding of cells. Six days later AP activity is measured.

FIG. 20. Mineralization by soluble Dll4.

MPC cells are infected with adenoviruses encoding either eGFP, BMP2 or H4-2. Three weeks after infection, cells are stained with VonKossa (upper panels) or Alizarin RedS stains (lower panels) and the resulting stainings photographed using white light microscopy.

FIG. 21. Mineralization by Notch Intracellular Domains (NICDs). MPC cells are infected with adenoviruses encoding either NICD-2. NICD-3, or NICD-4. Three weeks after infection, cells are stained with Alizarin RedS stains and the resulting stainings photographed using white light microscopy.

### EXAMPLES

### Example 1

### Library Construction

An arrayed human placental cDNA expression library in adenoviral vector format (placental PhenoSelect cDNA library) is constructed and screened using an alkaline phosphatase assay. Further details about the concept of arrayed adenoviral libraries can be found in WO 99/64582 (Arrayed adenoviral libraries for performing functional Genomics). The generation of the placental adenoviral cDNA library has been described elsewhere (European patent Application 01870038.5).

### Example 2

### Generation of a BMP-2 Control Virus

IMAGE clones harboring long ESTs for human BMP-2 are purchased from Incyte Genomics: IMAGE clones 2163129, 2189183 and 562997 with the Genbank numbers AI497606, AI537781 and AA086008, respectively. Bacterial stabs are streaked onto ampicillin containing agar plates and single colonies obtained after overnight growth at 37° C. are subjected to a colony PCR to verify the DNA inserts using primers over the start and stop codon of human BMP-2. The sequences of the respective primers are: 5′-GGTTGTGGGTGTCGCTAGTGGATCCCGC (SEQ ID NO: 11) and 5′-GCGAAGCTTACCATGGTGGCCGGGACCC (SEQ ID NO: 12). Bacterial colonies corresponding to PCR products with the expected length are picked, innoculated in liquid LB medium and grown overnight at 37° C. The plasmids are extracted from the selected colonies and inserts are isolated by PCR using the above-mentioned PCR primers. The resulting PCR products are digested with HindIII and BamHI restriction enzymes and ligated into the adenoviral vector pIPspAdapt6 previously digested with the same restriction enzymes. Plasmids containing the BMP-2 cDNA are isolated and the inserts are verified by sequence analysis for possible mutations incorporated during the PCR reaction. A clone containing a cDNA insert identical to the wild-type human BMP-2 cDNA is thus obtained and inserted into an adenoviral vector.

### Example 3

### Increase Infection Efficiency of Human MPCs Using Adenovirus-mediated Expression of hCAR

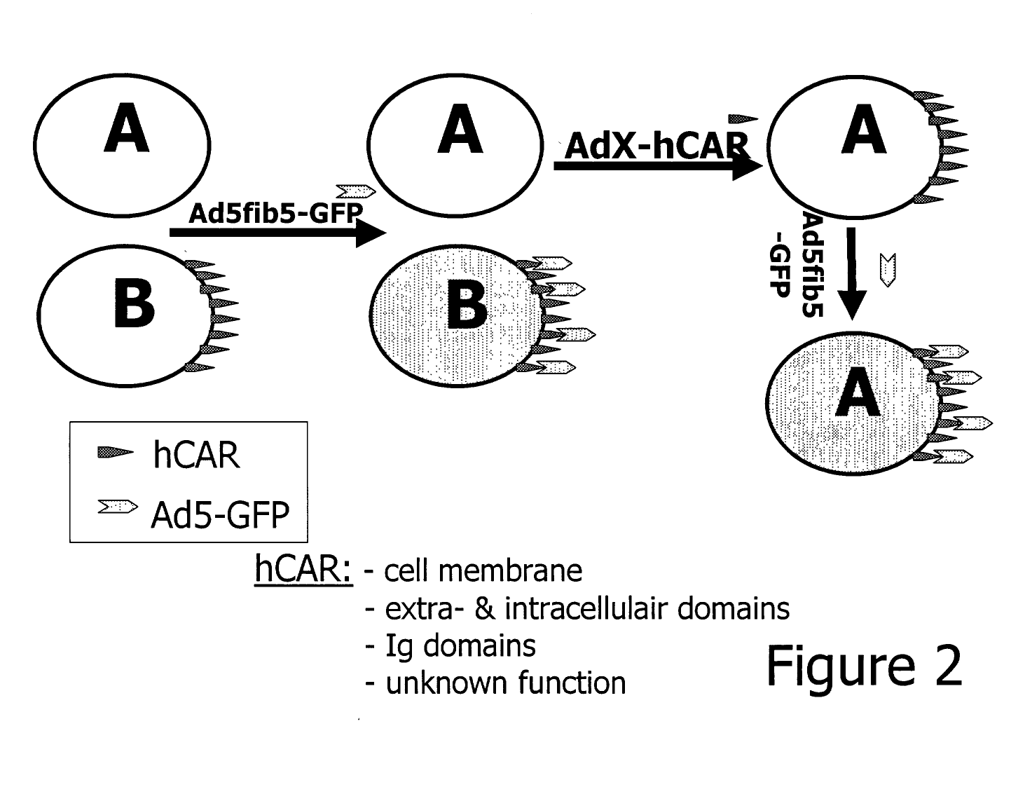

Mesenchymal progenitor cells (MPCs) are difficult to transduce with Ad5 virus. Adenoviral infection is initiated by the formation of complexes between the globular knob domain of the adenoviral fiber protein and a host cell receptor. The fiber receptor for Ad5 (and the other members of Adeno subgroups A, C, D, E and F) has been identified as the Coxsackievirus and Adenovirus Receptor (CAR) (Bergelson et al., 1997). Thus, cells that do not carry the CAR receptor or that express the receptor at very low basal levels are difficult to infect efficiently with any of these adenoviral vectors, including Ad5 viruses. One way to infect these cells more efficiently is to use an Ad virus from group B or to use a fiber variant (e.g., Ad5fib35 or Ad5fib51) that can enter the cell through a different receptor. Although this approach has proven to be quite successful, it cannot be used for the existing placental PhenoSelect™ cDNA library which has been produced in Ad5. Therefore an alternative strategy is devised wherein the cells are first infected with Ad5 or an Ad5 fiber variant expressing hCAR (human receptor) prior to infection with the placental PhenoSelect™ cDNA library viruses. The principle of the approach, which allows one to infect virtually every single cell line or polyclonal primary cell population, is shown in FIG. 2. Although the transduction efficiency of the hCAR containing Ad5 viruses (or variants) may be modest, sufficient hCAR receptor can be expressed on the surface of the MPCs after adenoviral mediated transfer to subsequently allow efficient infection with the library viruses.

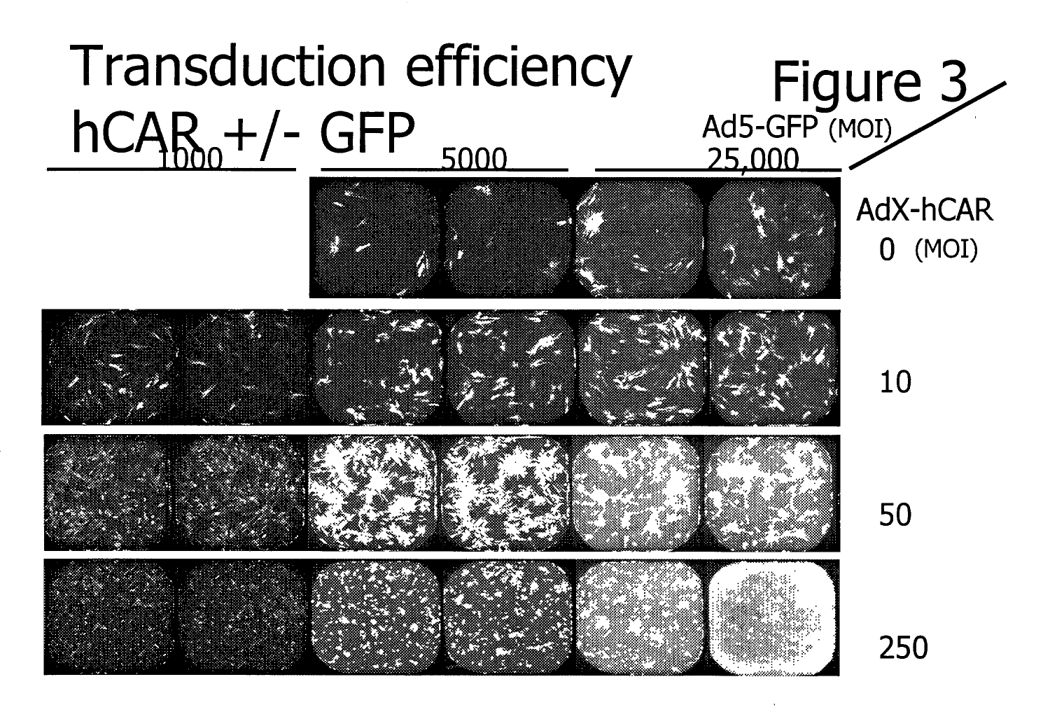

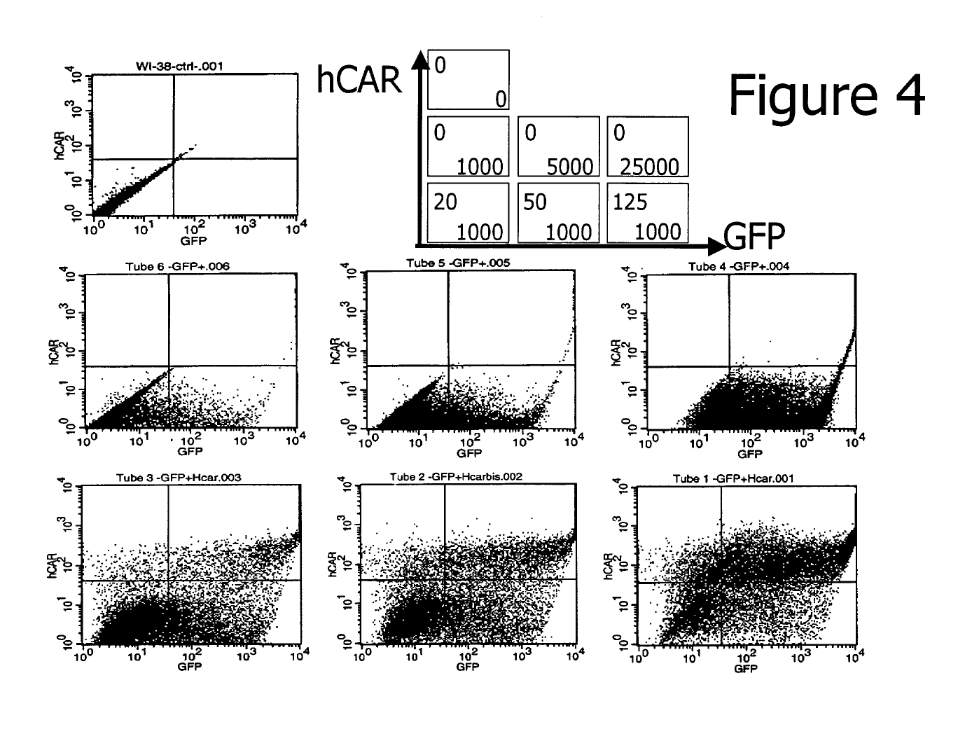

The procedure for infecting the MPCs with adenoviruses as described above is optimized as follows: MPCs are seeded in 384 well plate at a density of 1000 cells per well and infected using Ad5-hCAR at a multiplicity of infection (MOI) of 10, 50, or 250. Ad5C01-eGFP (Ad5-GFP) control virus is used at an MOI of 1000, 5000 or 25000. Infection efficiency is analyzed 6 days later using fluorescence microscopy (Zeiss Axiovert 25, 4× objective). For a single infection with Ad5C01-eGFP, high MOIs are required to infect the majority of MPCs (see FIG. 3). However, double infection using Ad5-hCAR significantly increases the infection efficiency of the Ad5C01-eGFP virus at MOI 1000 compared to the single infection. These data show the efficacy of using Ad5-hCAR double infections in order to increase infection efficiencies. The experiment is repeated for the MPC cells, seeded in 6 well plates. Six well plate formats are used to increase the number of cells to quantitatively analyze the Ad5-eGFP transfection efficiencies by fluorescence activated cell sorting (FACS). As is shown in FIG. 4, double infections of Ad5-eGFP and Ad5-hCAR show superior transfection efficiencies over single Ad5-eGFP infections in accordance with the results obtained in FIG. 3

In the double infections much lower Ad5-eGFP titers can be used to infect MPCs than in a single infection. This represents a substantial cost saving factor for functional genomics screens.

It is also determined that Ad5C15-hCAR and Ad5C20-hCAR increase the infection efficiency of Ad5(fib5) viruses on human MPCs at similar MOIs as Ad5(fib5)-hCAR (data not shown).

All the viruses described in this patent application have the Ad5 genome backbone with the E1A, E1B and E2A genes deleted and fiber variant 5. Only the viruses Ad5C15-hCAR and Ad5C20-hCAR, described in this paragraph, have a fiber modification (C15 or C20 instead of fib5) and do not have the E2A gene deleted in their genome.

### Example 4

### Development of a High-throughput Screening Method for the Detection of Endogenous Alkaline Phosphatase

Description of the Assay:

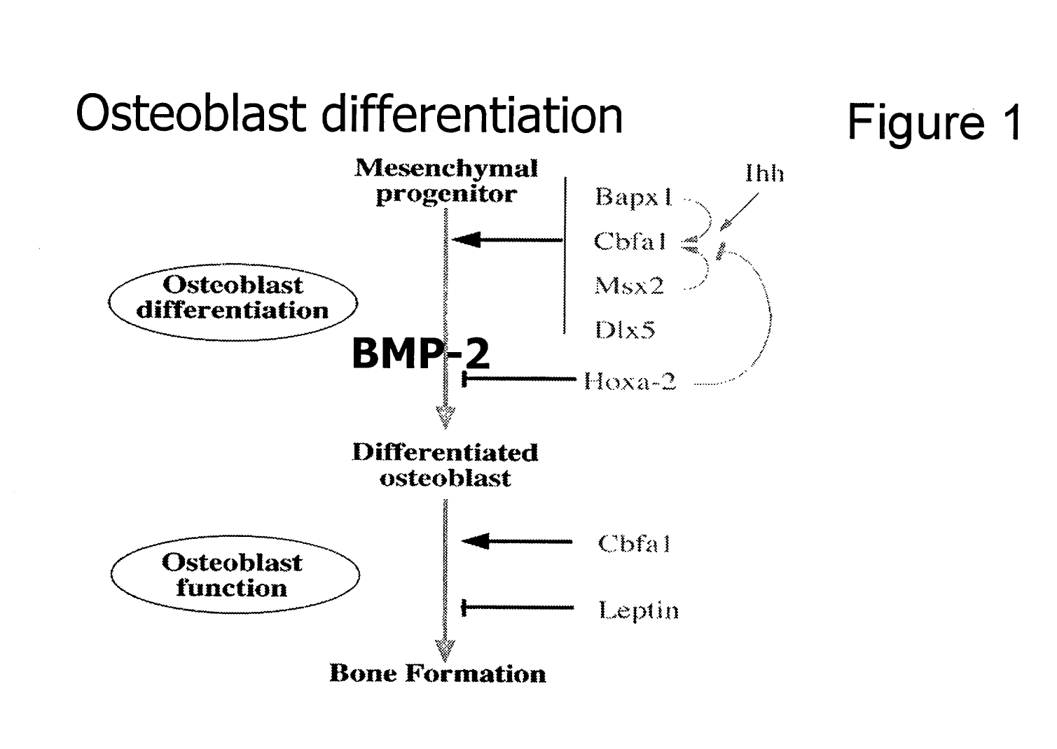

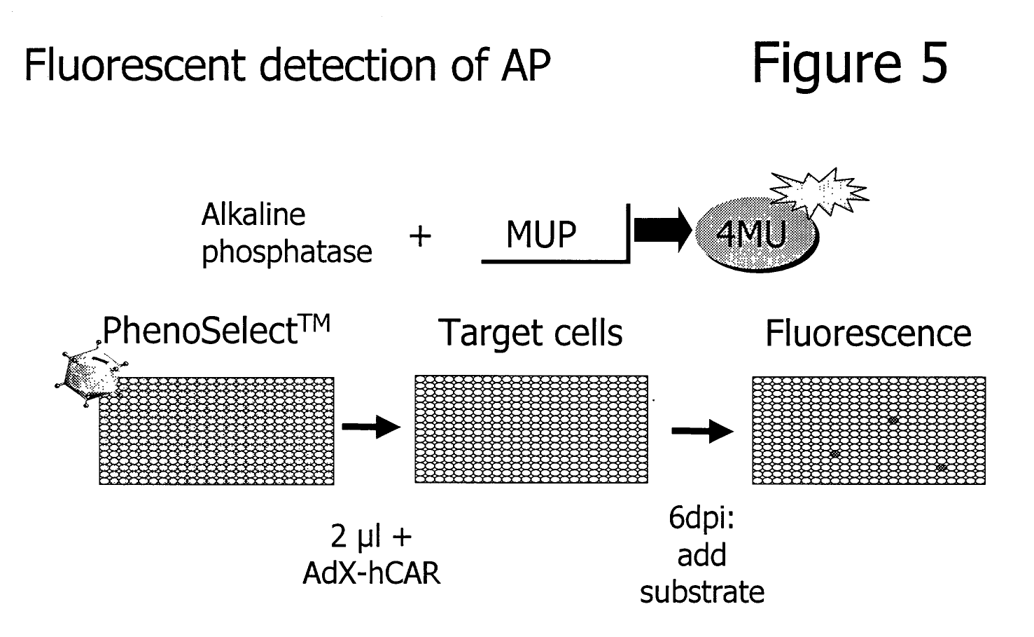

Mesenchymal precursor cells are determined to differentiate into osteoblasts in the presence of appropriate factors (e.g., BMP2, see FIG. 1). An assay to screen for such factors is developed by monitoring activity of alkaline phosphatase (AP) enzyme, an early marker in the osteoblast differentiation program. MPCs are seeded in 384 well plates and simultaneously co-infected one day later with Ad5fib5-hCAR and individual PhenoSelect viruses (as outlined in example 3). Six days post infection (dpi), cellular alkaline phosphatase activity is determined (FIG. 5).

Assay Set-up:

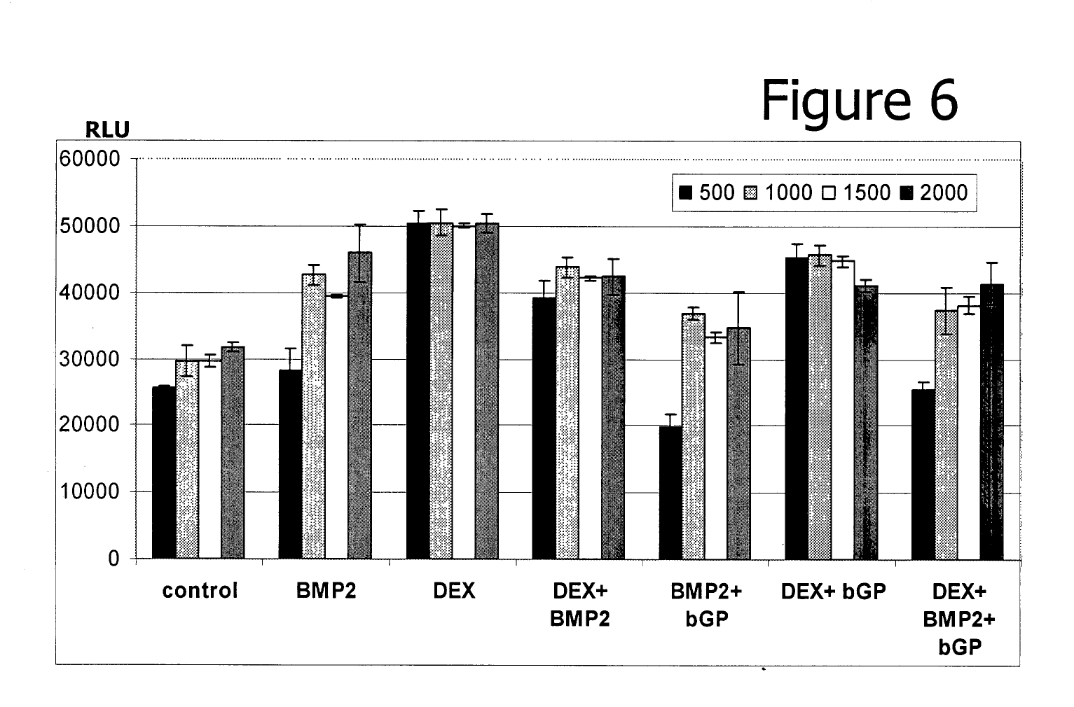

MPCs isolated from bone marrow of patients that have undergone prosthetic implantation/replacement surgery, are obtained from Isotis. The optimal cell density for plating the cells is determined by seeding the MPCs in black 384 well plates with clear bottom (Costar or Nunc) at a density of 500, 1000, 1500, or 2000 cells per well. One day after seeding the cells, solutions (obtained from Isotis as 100×stock solutions) containing factors that induce (Dexamethasone or recombinant human BMP-2) or accelerate (beta-glycerophosphate) osteoblast differentiation of MPCs are added to the medium either individually or in combination (see FIG. 6). Six days later, up regulation of endogenous AP activity is measured as follows: the conditioned medium is removed from the cells and the cells are washed once with PBS before the addition of 10 μl of a methylumbelliferyl heptaphosphate (MUP) solution (Sigma cat no M3168) to each well. The plates are then incubated at 37° C. for 30 minutes and the enzymatic reaction is stopped by the addition of 5 μL of 1M Na₂CO₃ solution to each well. The fluorescence generated upon cleavage of the MUP substrate by the AP activity is measured on a fluorescence plate reader (Fluostar, BMG). The relative fluorescence units (RLU) obtained from untreated cells and from cells treated with either dexamethasone (DEX), beta-glycerophosphate (bGP), recombinant human BMP2 (rhBMP2) or a combination of these factors, are compared (FIG. 6). Alkaline phosphatase activity is clearly upregulated by the dexamethasone and BMP2 treatments (FIG. 6). Treatment of the cells with beta-glycerophosphate or with a combination of beta-glycerophosphate and the other factors does not result in a (synergistic) upregulation of the endogenous AP activity. Because signals are weaker for cells seeded at a cell density of 500 as compared to those seeded at a higher density (1000, 1500, or 2000), the former density is considered too low. As the signals obtained for cell densities of 1000, 1500 or 2000 are similar, the minimal number of cells, i.e., 1000 cells per well, that allows for a robust detection of endogenous AP activity upregulation is used in further experiments.

Assay Validation Using Adenoviral Infections:

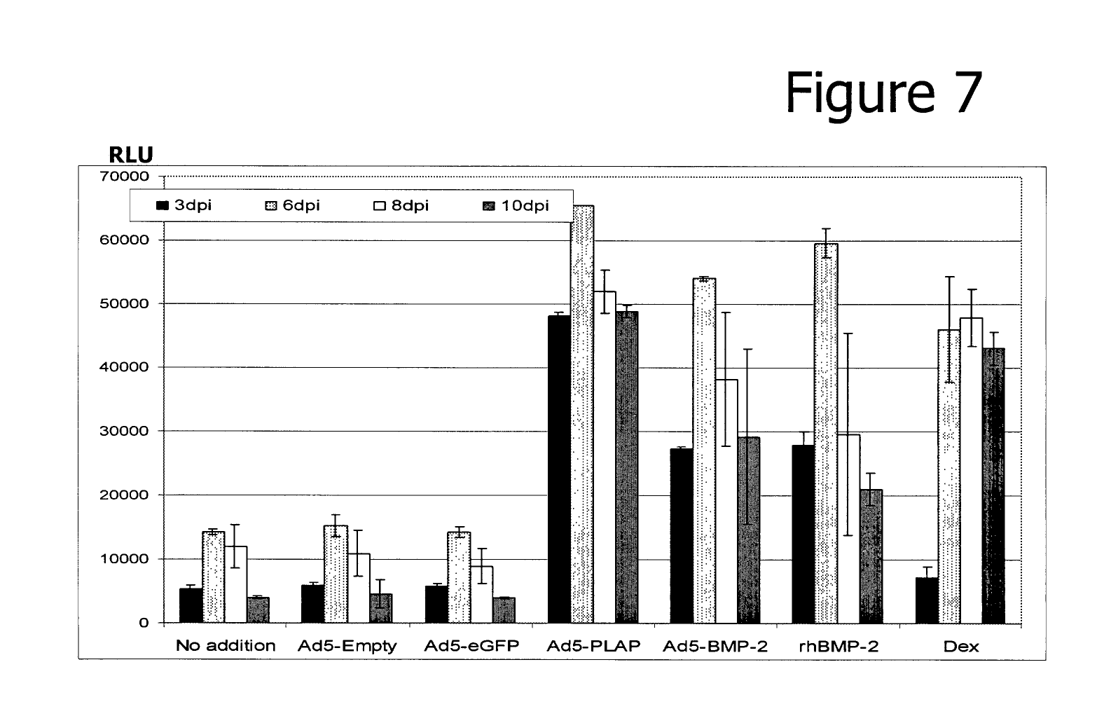

The assay is validated with bone morphogenic protein 2 virus (Ad5-BMP2) as a positive control. Ad5-eGFP or Ad5-empty virus are used as negative controls to evaluate background effecs and the effects of the viral infection on the cells, respectively. MPCs are seeded in 384 well plates at a density of 1000 cells per well and are infected 1 day later using a co-infection of either one of the negative or positive control viruses at an MOI of 1000 combined with Ad5-hCAR virus at an MOI of 100. A single infection with Ad5-hCAR at an MOI of 100 is included as a control. Endogenous AP activity is determined as described above at 3, 6, 8 and 10 days post infection. Ad5-BMP2 viruses clearly upregulated endogenous AP activity high above background (FIG. 7), demonstrating that viral-mediated BMP2 expression in MPCs elicits similar effects on induction of osteoblast differentiation as rhBMP2 or dexamethasone. Furthermore, endogenous AP activity becomes strongly upregulated 6 days post infection and does not increase further 8 or 10 dpi. The earliest time point for maximal detection is thus 6 dpi and this timepoint is used to read-out AP activity in further experiments.

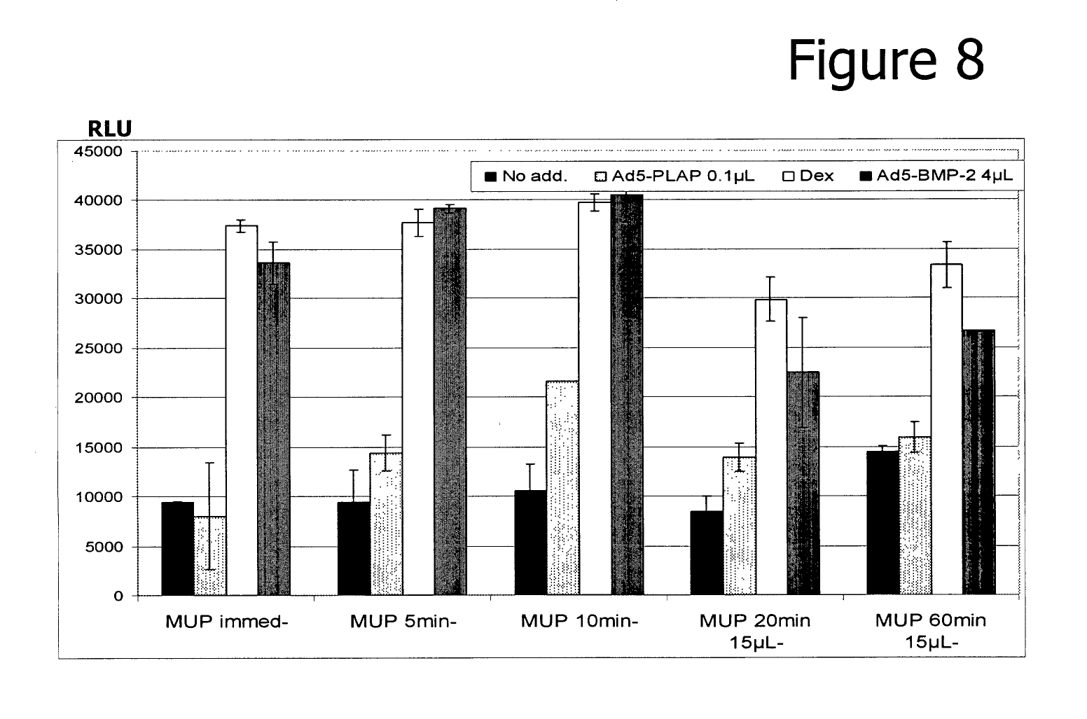

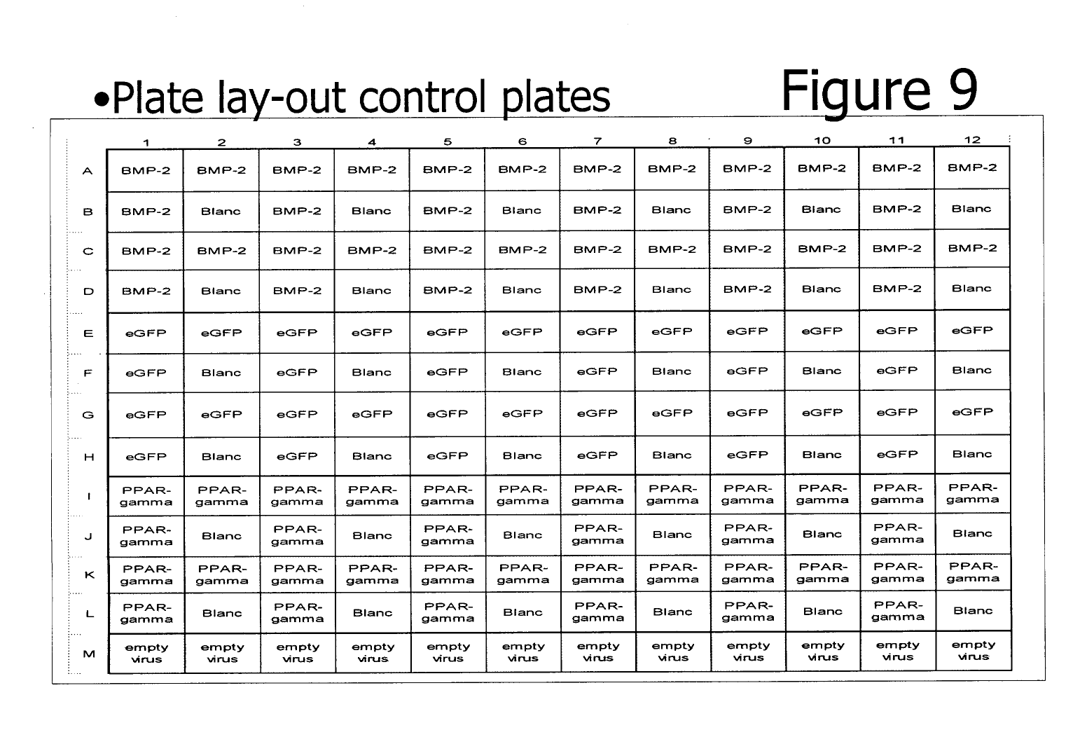

Using Ad5-BMP2 as a positive control, the optimal incubation time for the MUP reagent added to the MPCs 6 dpi, are determined. Incubation times of 0, 5, 10, 20 and 60 minutes are compared at 37° C. (FIG. 8). Signal to noise ratios are comparable after 0, 5, 10 and 20 min. incubation but dropped for the 60 min. time point. A 15 min. incubation time is considered most suitable for performing the above described protocol in a high throughput screening mode.

Note that in the experiment described in FIG. 8, the reaction is not stopped by the additionn of the Na₂CO₃ solution. As this and other experiments have demonstrated, there is no clear benefit in adding the stop solution as long as the fluorescent signals are read within 45 min after addition of the MUP substrate. Furthermore, washing the cells prior to adding MUP does not yield better signal to noise ratios as compared to washing the cells. The wash step wash is therefore omitted in all later experiments.

### Example 5

### Pilot AP Screen with 6500 Adenoviruses in 384 Well Format

In order to screen a library of tens of thousands of arrayed adenoviruses the assay as described in example 4 is adapted for high-throughput screening.

Preparation of Control Plates

96 well control plates containing different control viruses transducing either BMP2, eGFP, PPAR-gamma, or luciferase transgenes or empty virus (defined as the virus without a transgene in its genome) are prepared. The lay-out of the control plate is shown in FIG. 9. These viruses are propagated as described for the library viruses. On day 0, 22,500 PerC6 cells are seeded in each well of a 96 well plate in 200 μl PerC6 medium. On day one, 1 μL of virus stock is added to the wells of this plate. Several days later, when full cytopathic effect (CPE) is observed in every virus-containg well, cells are subjected to one freeze-thaw cycle (at −80° C. and room temperature, respectively) and aliquots of the the resulting crude lysates containing the propagated viruses are deposited in new 96 well plates. Plates are sealed (seal obtained from Nunc, Cat no 236366) and stored at −80° C. until futher use.

Protocol for Screening the PhenoSelect Library

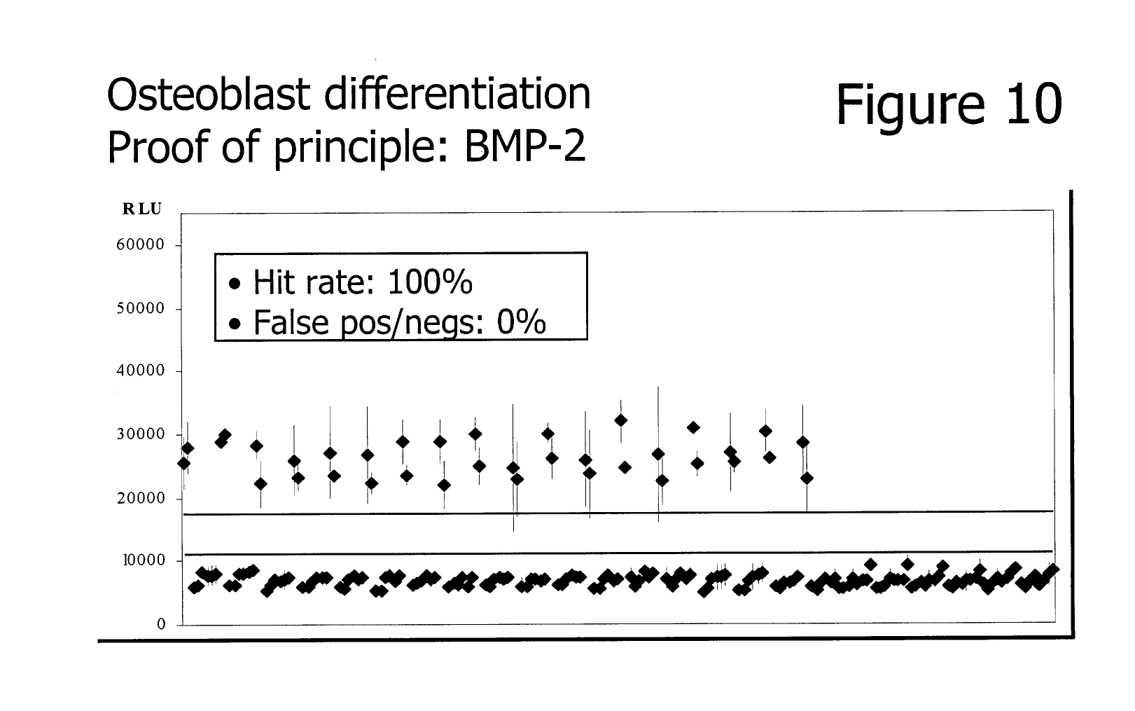

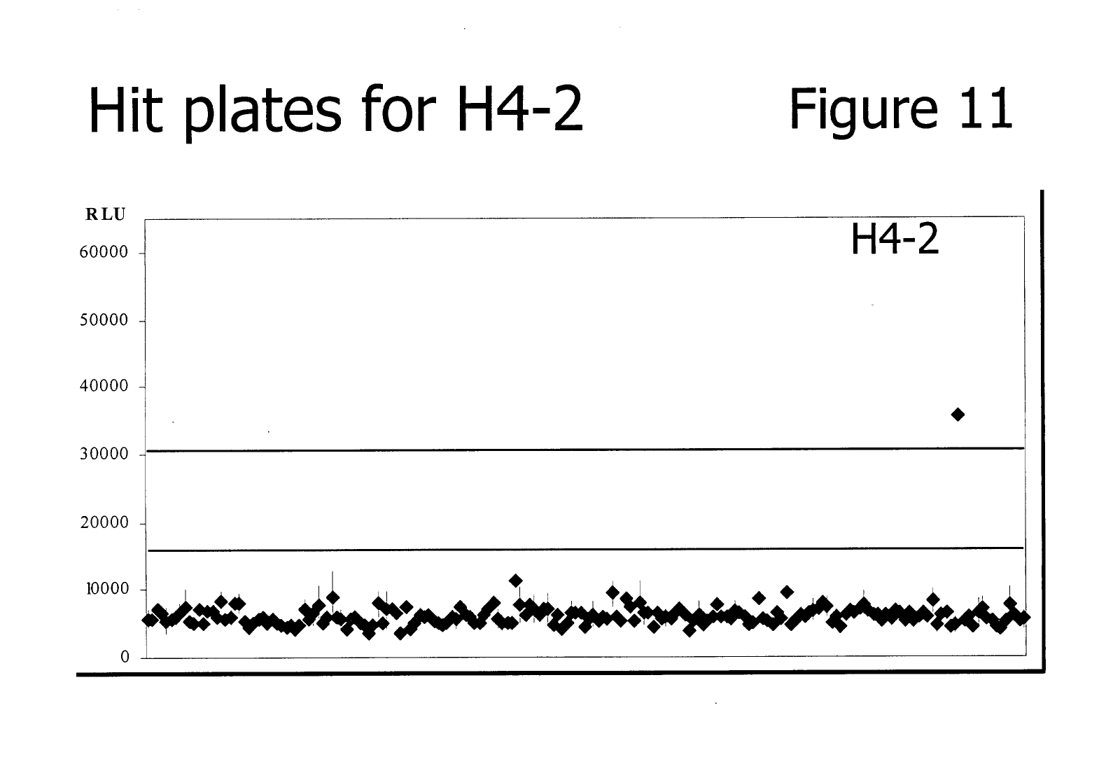

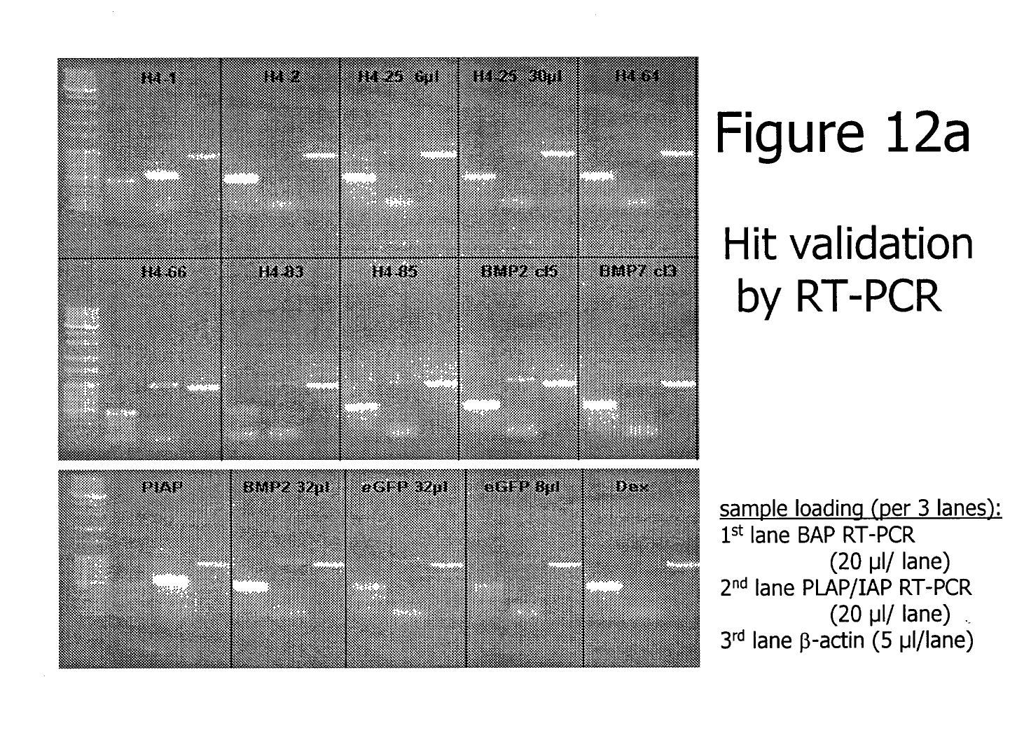

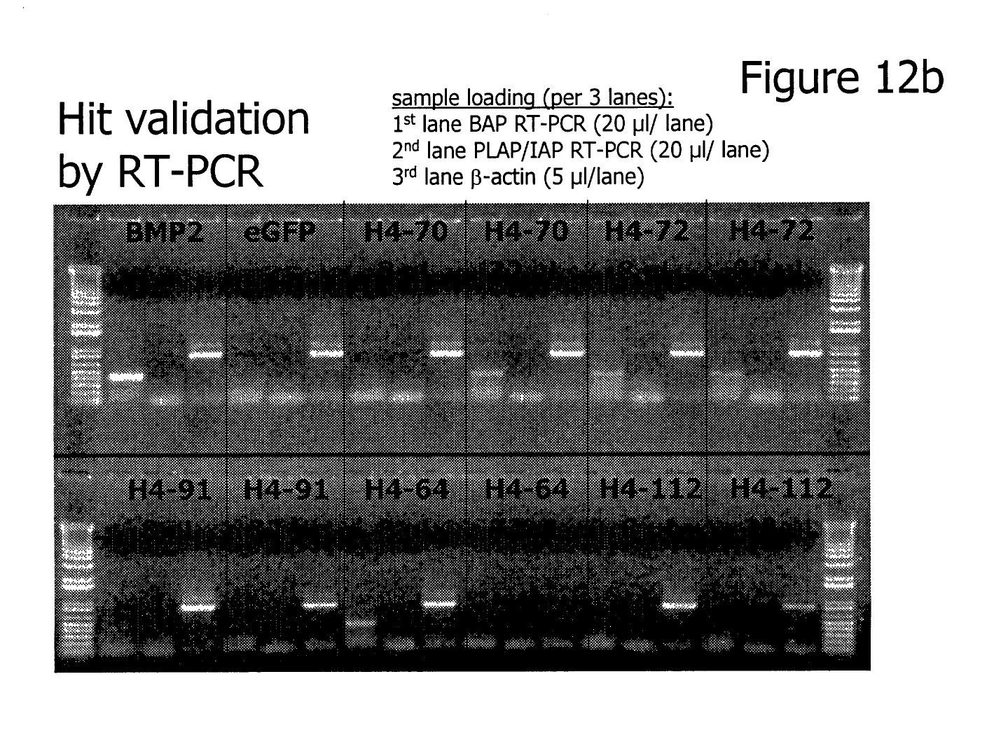

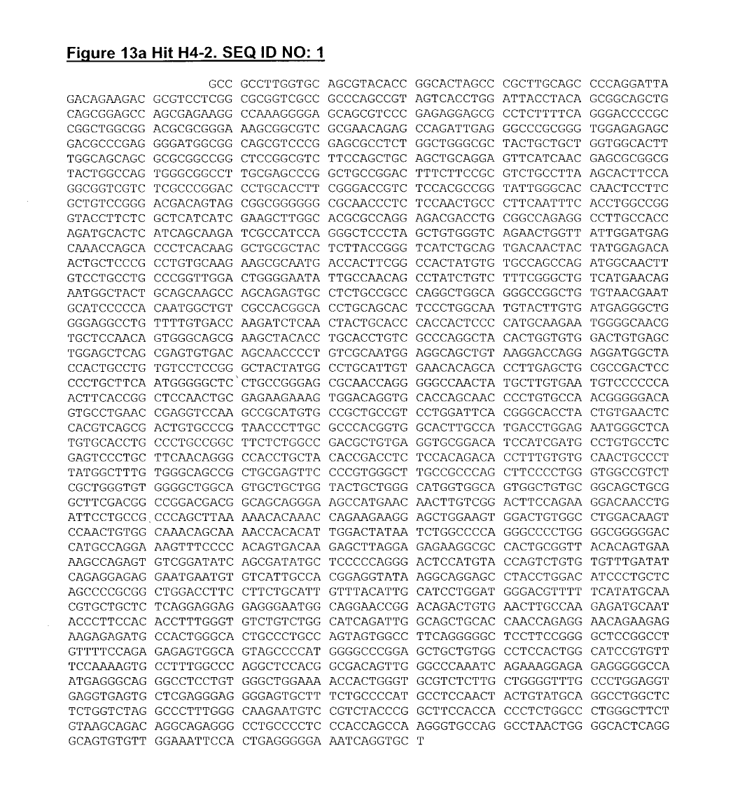

On day 0, MPC cells are seeded in black 384 well plates with clear bottom (Costar or Nunc) in 60 μl medium at a density of 1000 cells per well . One day later, 10 μl Ad5-hCAR virus (10⁷ viral particles per ml) is transferred with the aid of a 96 channel dispenser (Hydra290, Robbins Scientific) from the wells of a 96 well Ad5-hCAR viral stock plate to each of the MPC containing wells of the 384 well plates. On the same day, control viruses or viruses from the PhenoSelect library are also added to the MPCs in the 384 well plates. Plates harboring control viruses or PhenoSelect library viruses are allowed to thaw at room temperature and two μl of each virus (estimated average of 2×10⁹ viral particals per ml) is then transferred with the aid of a 96 channel dispensor (Hydra100, Robbins Scientific) to individual MPC containing wells. The viruses from the control plates are screened in quadruplicate. The viruses from the PhenoSelect library are screened in duplicate. The plates containing the freshly infected cells are then incubated at 37° C. Six days post infection all conditioned medium is aspirated before 15 μl MUP is added to each well. The plates are incubated for 15 min at 37° C. and monitored for AP activity using a fluorescence plate reader (Fluostar, BMG). The results obtained in a typical experiment for a control plate are shown in FIG. 10.

Data Analysis

The data obtained from the FluoStar are analyzed as follows: for control plates, the mean of the relative fluorescence units obtained for all the viruses except Ad5-BMP2 virus is calculated. The standard deviation for these data points is also calculated. The cut-off value for hit calling is then pre-set at the mean plus 3 times the standard deviation. Values above this cut-off value have more than a 99% statistical chance of scoring truly above background.

All the signals obtained for BMP2 viruses are clearly above this cut-off value (FIG. 10), showing the validity of this approach. Thus, no false positives or false negatives are seen when processing the control plates according to the library screenings protocol.

When viruses from the PhenoSelect library are screened, the background is calculated by taking the mean of all data points. As the mean varies slightly from plate to plate within the same screening round, two cut-off values are assessed; one set at the mean calculated for all datapoints in a given plate plus three times standard deviation and one set at the mean calculated for all data points over all plates in the screening round plus 3 times standard deviation.

As the hit rate is still rather high and a large number of false positives are picked up when using 3 times standard deviation, cut-off values are later tightened by using 3.4 times the standard deviation calculated for all data points.

Upon screening 6500 PhenoSelect viruses in the AP assay, 65 viruses score above the cut-off value. Ten of these 65 viruses score higher than the mean plus 4 times the standard deviation.

FosB, a factor known to enhance bone formation (Sabatakos et al., 2000) is identified as one of the stronger scoring hits (values at the mean plus 9 times the standard deviation) in the assay. DeltaFosB, a naturally occurring splice variant generated from the full-length FosB mRNA, is previously described by Sabatakos and colleagues to be a potent inducer of osteoblast proliferation and differentiation in vivo in mice thus corroborating our finding. Identification of FosB, an established player in osteoblast differentiation further validates our assay. That this cDNA is found in a rather modest screen of 6500 viruses also demonstrates the speed with which the high-throughput screenings method can turn up gene products that play important roles in bone cell differentiation

### Example 6

### Rescreen Hits from Pilot AP Screen

The 65 viruses identified in the AP screen described in example 5 are repropagated from the original viral stocks. This is achieved by infecting PerC.6/E2A cells seeded in 96 well plates at a density of 4×10⁴ cells per well (200 μl of DMEM+10% FBS) with 1 μl of the original viral stock solution from the PhenoSelect library plates. After incubation of these plates for six days, CPE is observed in most wells and the plates are transferred from the 37° C. incubator to a −80° C. freezer. Aliquots are made of these viruses upon thawing in V-bottom 96 well plates and then rescreened in the AP screen. The latter is performed as described under Example 5. From the original hit viruses, 5 viruses score positive in the rescreen.

### Example 7

### Sequence Identification of Validated Hits

For sequencing analysis, fragments of the cDNAs expressed by the hit adenoviruses are amplified by PCR using primers complementary to vector sequences flanking the multiple cloning site of the pAdapt plasmid. The following protocol is applied to obtain these PCR fragments. PerC6/E2A cells are seeded in 96 well plates at a density of 40,000 cells per well in 180 μl PerC6/E2A medium. Cells are then incubated overnight at 39° C. in a 10% CO₂ humidified incubator. One day later, cells are infected with 2 μl of crude cell lysates from the repropagated ‘hit viruses’. Cells are incubated further at 34° C., 10% CO₂ until appearance of CPE (as revealed by the swelling and rounding up of the cells, typically 2 to 3 days post infection). The supernatant is then removed from the cells and exchanged for 50 μl lysis buffer (1×Expand High Fidelity buffer with MgCl₂ (Roche Molecular Biochemicals, Cat. No 1332465) supplemented with 1 mg/ml proteinase K (Roche Molecular Biochemicals, Cat No 745 723) and 0.45% Tween-20 (Roche Molecular Biochemicals, Cat No 1335465). After mixing, cell lysates are transferred to sterile micro centrifuge tubes and incubated at 55° C. for 2 h followed by a 15 min inactivation step at 95° C. 5 μl of the cell lysates is then added to a PCR master mix composed of 5 μl 10×Expand High Fidelity buffer with MgCl₂, 1 μl of dNTP mix (10 mM for each dNTP), 1 μl of pClip-FOR primer (10 μM stock, sequence: 5′ GGT GGG AGG TCT ATA TAA GC, SEQ ID NO: 13), 1 μl of pAdapt-REV primer (10 μM stock, sequence: 5′ GGA CAA ACC ACA ACT AGA ATG C, SEQ ID NO: 14), 0.75 μl of Expand High Fidelity DNA polymerase (3.5 U/μl, Roche Molecular Biochemicals) and 36.25 μl of H₂O. PCR is performed in a PE Biosystems GeneAmp PCR system 9700 as follows: the PCR mixture (50 μl in total) is incubated at 95° C. for 5 min; at 95° C. for 30 sec; 55° C. for 30 sec; 68° C. for 4 min, and this is repeated for 35 cycles. A final incubation at 68°°C. is applied for 7 min. 10 μl of the PCR mixture is mixed with 2 μl of 6×gel loading buffer, loaded on a 0.8% agarose gel containing 0.5 μg/ml ethidium bromide to resolve the amplification products. The size of the amplified fragments are estimated from from a standard DNA ladder loaded on the same gel. .

### Example 8

### Validation of Hits from Osteoblast Differentiation Screen by RT-PCR

RNA Extraction

Total RNA from mesenchymal progenitor cells (MPC) is extracted 7 days post infection using TRIzol® reagent (Life Technologies) according to the manufacturer's recommendations.

Briefly, cells (24-well plate) are homogenized in 300 μl TRIzol® reagent. Phases are separated by the addition of chloroform and RNA is iso-propanol-precipitated from the aqueous phase. The RNA pellet is washed with 70% ethanol, air-dried and dissolved in 50 μl DEPC treated H₂O.

The concentration of all RNA extracts is then determined using RiboGreen RNA Quantitation Reagent (Molecular Probes) and diluted to a final concentration of 40 ng/μl.

Reverse Transcription PCR

Gene specific primers are designed to ALPL (human alkaline phosphatase liver/bone/kidney) and to ALPI (human alkaline phosphatase intestinal), ALPP (human alkaline phosphatase placental (PLAP)), ALPPL2 (human alkaline phosphatase placental-like). ALPI, ALPP and ALPPL2 are highly similar at the nucleotide level and are PCR amplified with one degenerate primer pair.

Y=C or T, M=A or C, K=G or T, S=C or G.

| | |
|---|---|
| BAP-FOR: | |
| GTGTGGGGTGAAGGCCAATGAGGGC | (SEQ ID NO:15) |
| BAP-REV: | |
| TCTTCCAGGTGTCAACGAGGTCCAGG | (SEQ ID NO:16) |
| PLAP/IAP-FOR: | |
| GTGCGGGGTCAAGGSCAACTTCCAG | (SEQ ID NO:17) |
| PLAP/IAP-REV: | |
| YCACAGACKGGTCCAGGGAMGCCTG | (SEQ ID NO:18) |

Human β-actin primers (Clontech) are used to check for RNA integrity.

RT-PCR is the most sensitive technique to determine the presence or absence of RNA templates. The Titan One Tube RT-PCR kit (Roche Molecular Biochemicals cat 1 855 476) is a one step reaction system based on AMV for first strand synthesis and Expand High Fidelity enzyme mixture for the PCR step. The protocol is followed as essentially described by the manufacturer.

Mix 1

| | |
|---|---|
| H2O DEPC-treated) | 7125 |
| dNTP mix | |
| 2.5 mM each | 2 |
| DTT | 1.25 |
| RNase inh 40U/μl | 0.125 |
| FOR 10 μM | 0.5 |
| REV 10 μM | 0.5 |
| template (*) | 1 |

Mix 2

| | |
|---|---|
| H2O (DEPC-treated) | 7 |
| 5 × RT-PCR buffer | 5 |
| Enzyme mix | 0.5 |

RT-PCR cycle program

| | | |
|---|---|---|
| 50° C. | 30 min | |
| 94° C. | 2 min | |
| 94° C. | 30 s | |
| 60° C. | 30 s | 10 x |
| 68° C. | 60 s | |
| 94° C. | 30 s | |
| 60° C. | 30 s | 25 x |
| 68° C. | 90 s | |
| 68° C. | 7 min | |
| 10° C. | hold | |

template (*): RNA @40 ng/μl for BAP and PLAP PCR, RNA @ 0.08 ng/μl for β-actin

Conclusions

The above protocol is run for negative and positive controls (eGFP, BMP-2, BMP-7, PLAP and dexamethasone) and hit H4-2. Increased BAP expression levels (above endogenous BAP expression) are detected in total RNA samples extracted from MPCs infected with H4-2. An approximately 1 kb PLAP fragment detected in some samples is generated from genomic DNA that contaminated the RNA extracts. RNA samples are not DNase treated.

### Example 9

### Validation of Hits for Activity as Secreted Proteins

Biopharmaceuticals are secreted human proteins with a specific and strong physiological action. As they are natural products they are valuable medicines for therapeutic intervention. Hits coming out of the high-throughput BAP screen can be used as biopharmaceuticals if they can be delivered in vivo or ex vivo and if they can induce osteoblast differentiation.

In order to analyze the potential of the hits identified in the high-throughput BAP screen as putative biopharmaceuticals the following experiments are performed: cells, hereafter termed “producer cells”, are infected with control viruses that either induce or do not induce osteoblast differentiation. The conditioned medium is harvested 3 or 4 days post infection (dpi) and added to freshly seeded MPC cells. If the conditioned medium contains secreted proteins that induce osteoblast differentiation, these proteins are identified by measuring the alkaline phosphatase activity (as described under examples 4 and 5) 6 days after transfer of the conditioned medium from the producer cells to the MPCs

HeLa and U2OS producer cells are cultured in DMEM supplemented with 10% FBS. The cells are plated in 384 well plates in 60-μl medium at a density of 5000 cells per well. Four hours later, the cells are infected with 1 μl of control (Ad5C01-BMP2, BMP7, FosB, PLAP, LacZ, luciferase or eGFP) or propagated hit viruses. Two or 3 days later, MPC cells are seeded in 384 well plates in 30 μl of medium at a density of 900 cells per well. One day after seeding the MPCs, 40 μl of the conditioned medium harvested from the HeLa or U2OS producer cells is transferred with the aid of a 96-channel dispenser (Hydra100, Robbins Scientific) to the corresponding MPC-containing cells of the 384 well plate . Six days later, the cellular alkaline phosphatase activity is determined on the MPCs as previously described.

### Example 10

### Features of H4-2

The features of H4-2, identified as an inducer of osteoblast differentiation in the assay described in example 9 are described below.

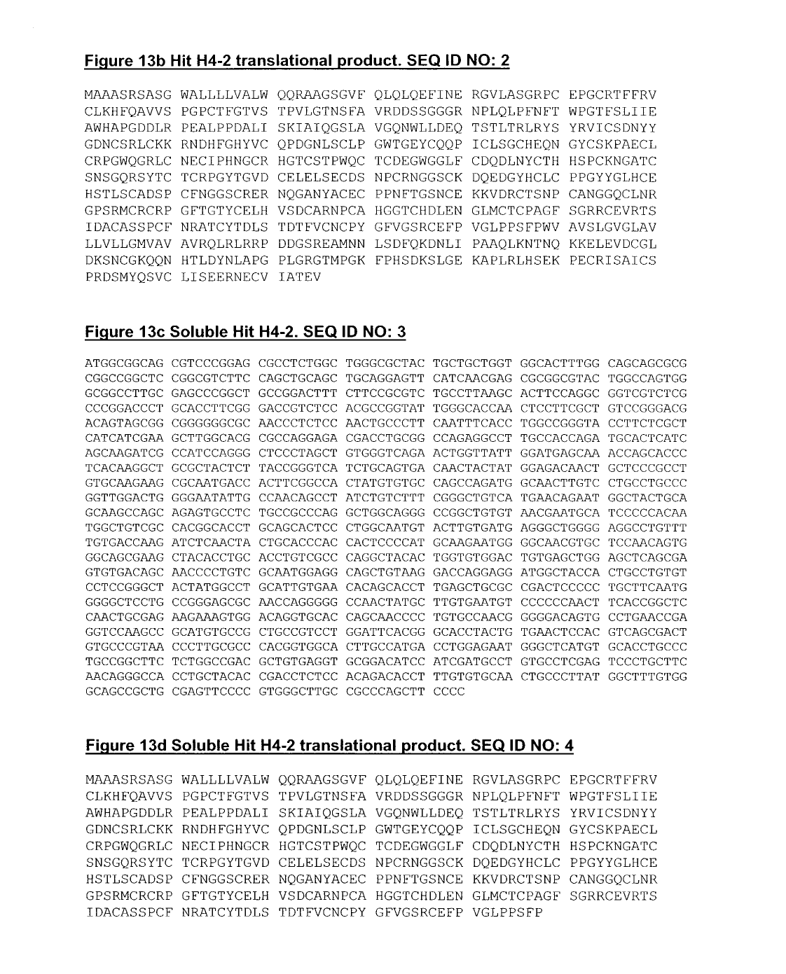

The H4-2 cDNA insert sequence (FIG. 13a, SEQ ID NO: 1) corresponds to human Delta2/Dll4, a presumed Notch ligand. This cDNA is identical to Homo sapiens hdelta2 for notch ligand delta-2, as described in GenBank record with accession number AB036931. This gene is also referred to as Homo sapiens delta-like 4 homologue (Drosophila) (DLL4), as described in GenBank record NM_{—}019074. In comparison with the AB036931 sequence, H4-2 is truncated at the 3′ end due to an internal NotI restriction site in the gene. Nevertheless, the full coding sequence is preserved in H4-2.

Based on the position of the open reading frames (ORFS) on the direct strand in relation to the promoter sequence, and the sequence similarity with published sequences, ORF number 10 most likely describes the coding sequence of this cDNA. ORF Number 10 on the direct strand extends from base 277 to base 2334. The protein encoded by ORF 10 matches perfectly with the protein sequence of GenBank record NP_{—}061947 (FIG. 13b, SEQ ID NO: 2).

From GenBank record NP_{—}061947, the following annotation is obtained: This gene is a homologue of the Drosophila delta gene. The delta gene family encodes Notch ligands that are characterized by a DSL domain, EGF repeats, and a transmembrane domain.

Two research papers provide some information on the expression pattern of this cDNA in mice: strong expression in heart and lung, weaker in skeletal muscle and kidney and still weaker but detectable expression in brain and liver (Krebs et al., 2000; Shutter et al., 2000). However, there is no evidence for the function of Delta2/Dll4 in murine or human organisms.

Therefore, as the invention shows that Delta2/Dll4 plays a role in osteoblast differentiation and possibly in bone homeostasis in general, the finding, as disclosed in the present invention provides novel and unexpected insights in regulators of bone remodeling.

### Example 11

### Dll4 (H4-2) as an inducer of osteoblast differentiation

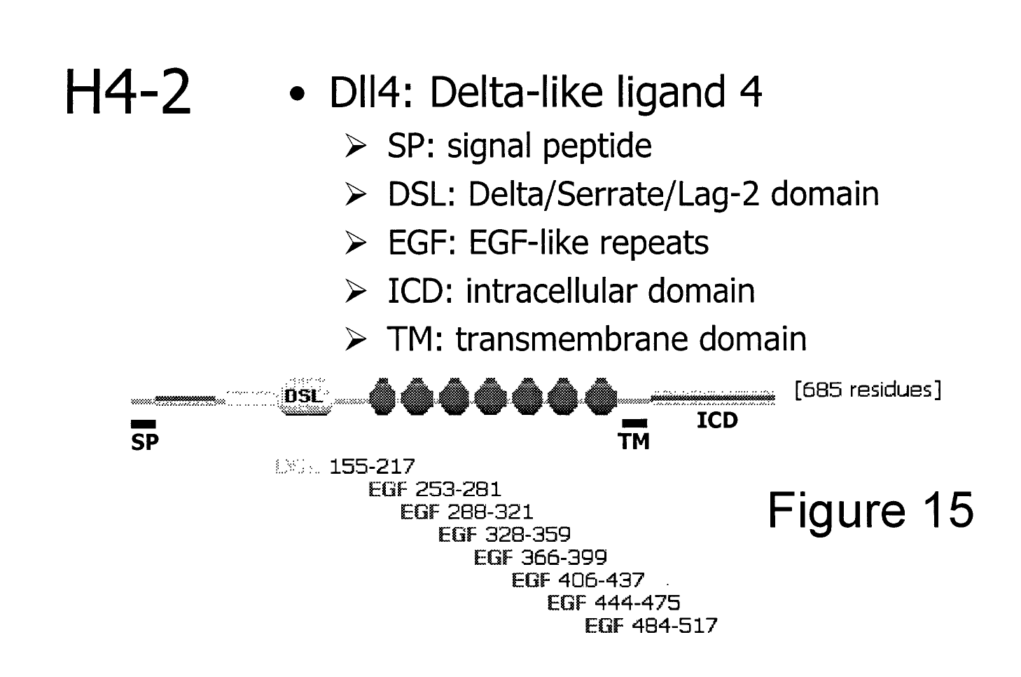

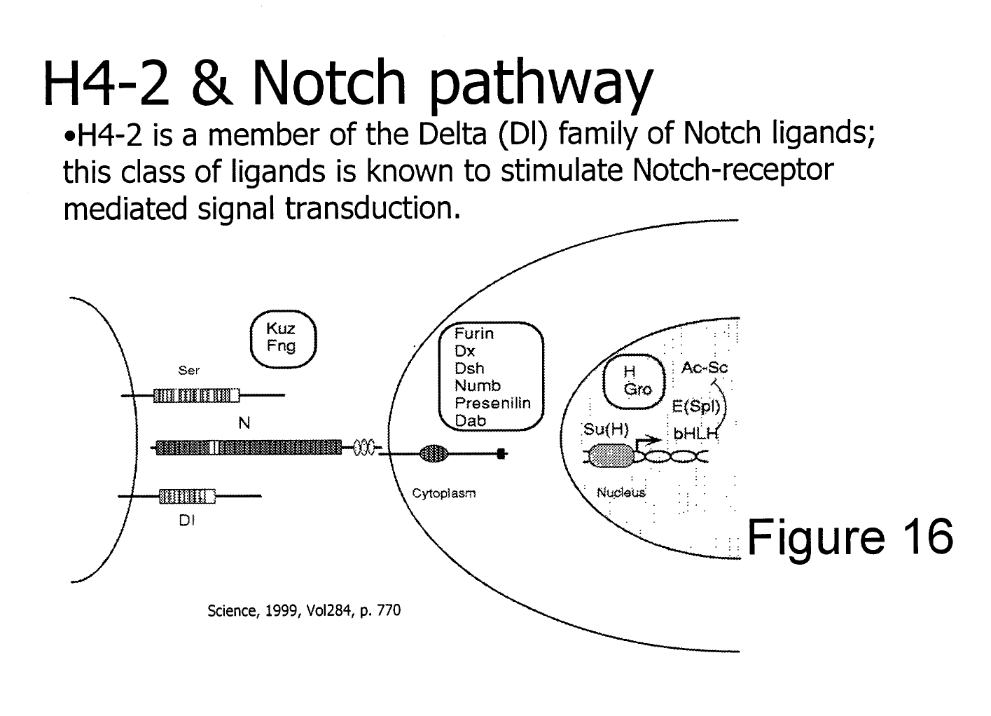

The H4-2 cDNA encodes the full-length Dll4 protein. Dll4 is presumed to be a Notch ligand, as it is a member of the Delta family of Notch ligands. Dll4 harbors the DSL and EGF-repeat domains in its extracellular region, characteristic of Notch ligands (see FIG. 15). Notch ligands induce Notch-intracellular-domain (NICD) mediated signal transduction.

Dll4 Up-regulates BAP and not PLAP or IAP

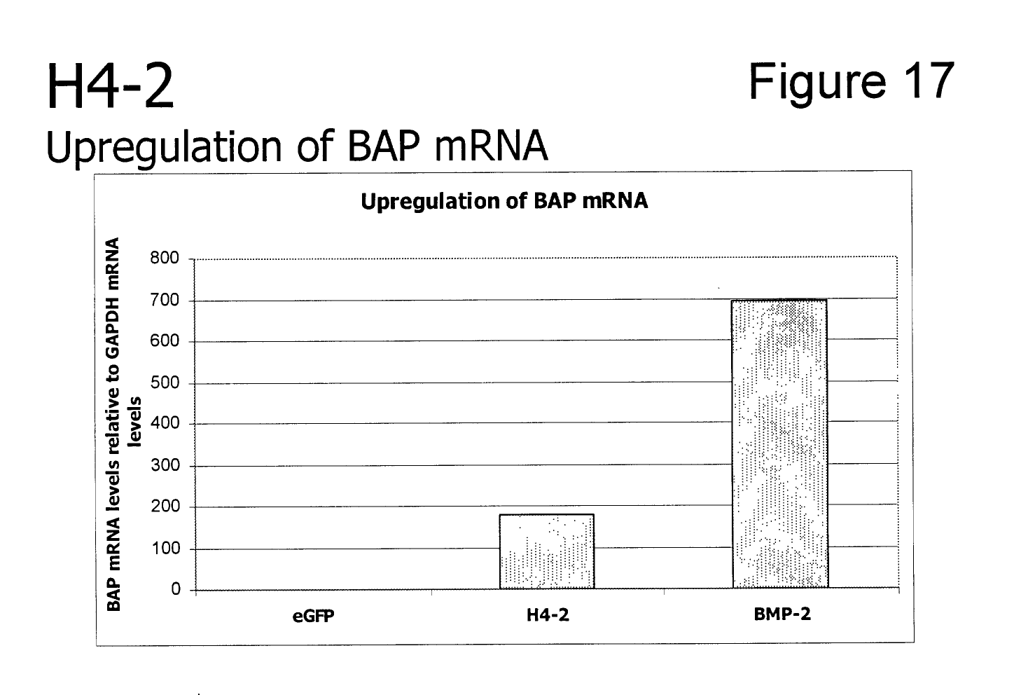

Real-time PCR data show that Dll4 specifically up-regulates bone alkaline phosphatase (BAP) mRNA (FIG. 17) and not placental (PLAP) or intestinal alkaline phosphatase (IAP) mRNAs (data not shown), indicating that Dll4 has osteogenic potential.

Mineralization

The process of osteogenesis consists of several successive events. During the initial phases of osteogenesis, bone alkaline phosphatase (BAP) becomes up regulated. As there are 4 alkaline phosphatases and only one is bone specific, alkaline phosphatase activity per se is not a fail-safe osteogenic marker. It is therefore important to look at more specific events occurring in later stages of osteogenesis such as mineralization.

Bone tissue consists of cells embedded in a matrix of organic materials (e.g., collagen) and inorganic materials (Ca²⁺ and phosphate). Bone mineralization is shown in vitro by staining differentiated bone cells for the matrix they deposited. The Von Kossa and Alizarin RedS stains allow the visualization of deposited phosphate and calcium, respectively.

Primary human MPCs are seeded in a 6 well plate (Costar or Nunc) at a density of 50,000 cells per well. MPCs are co-infected one day later with Ad5C15-hCAR (at MOI 1000) and Ad5C01-control or hit virus (at MOIs of 500, 1500, or 4500). Medium, supplemented with 100 μg/ml L-ascorbate and 10 mM beta-glycerophosphate, is refreshed 3 times a week. BMP-2 and eGFP viruses are used as a positive and negative control, respectively. 20 to 30 days after the start of the experiment, cells are stained with Von Kossa stain or with Alizarin RedS stain.

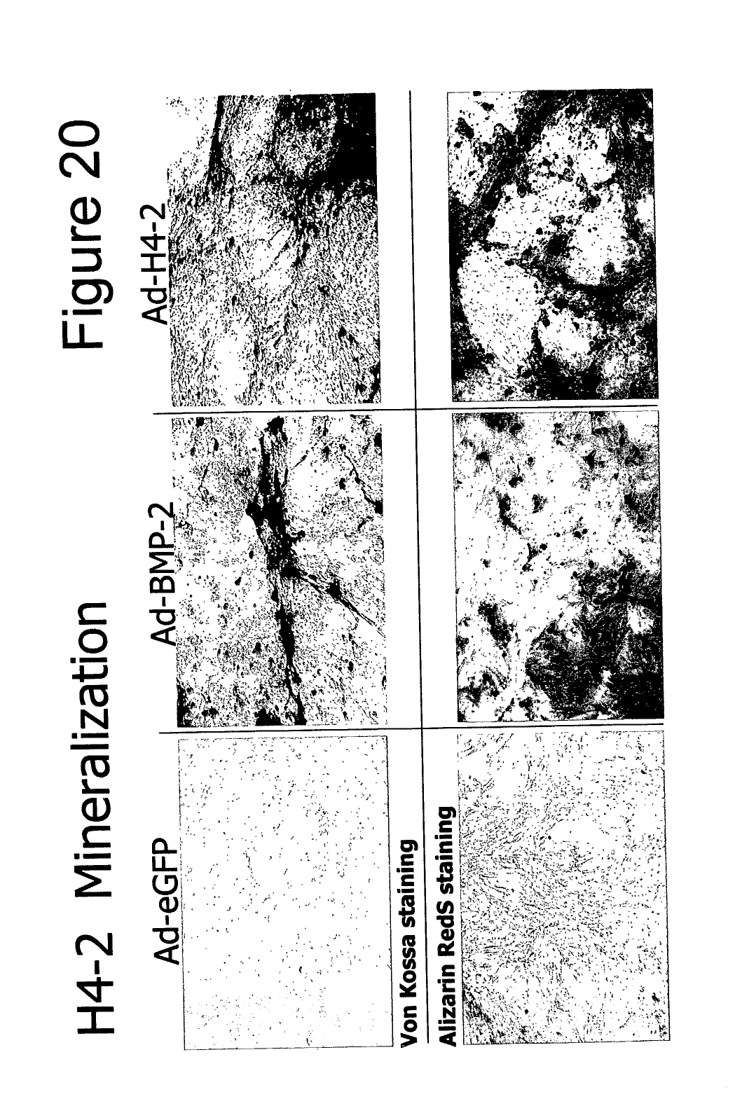

For the Von Kossa staining cells are first washed 3 times with PBS then fixed with 4% paraformaldehyde for 4 hours at 4° C. and washed again 3 times with distilled water. After addition of 5% silver nitrate solution, the plates are exposed to UV light for 1 hour. The cells are rinsed 3 times with distilled water before and after the residual silver nitrate is neutralized with 2.5% sodium thiosulphate. Phosphate deposits become visible as black clusters and are easily photographed under white light microscopy (FIG. 20, upper panels).

The Alizarin RedS staining is carried out as follows: cells are washed twice with PBS, fixed with 10% paraformaldehyde for 45 minutes at 4° C. and washed 3 times with PBS. Cells are incubated with 40 mM aqueous Alizarin RedS solution, pH 4.1-4.3 for 10 minutes followed by 5 washes with distilled water. Staining is evaluated and photographed using white light (FIG. 20, lower panels).

### Example 12

### Dll4 Mutants

p6Dll4 His

A soluble human Dll4 mutant lacking the transmembrane domain as a result of a truncation after Proline 528 is constructed. A C-terminal 6 histidine amino acid tag is also added to the protein to facilitate the detection and purification of the soluble Dll4 protein variant.

To construct the Dll4 mutant, the cDNA from the H4-2 clone is used as the template together with specific primers on pIpspAdapt6-H4-2 plasmid DNA in a PCR reaction with PfuTurbo polymerase (hifidelity, Stratagene) and.

The forward primer 5′AACCGAGGTCCAAGCCGCATGTGCC 3′ (SEQ ID NO: 19) anneals specifically with nucleotides 1255 to 1279 of the Dll4 coding sequence. The reverse primer 5′CGCGGATCCTAATGGTGATGGTGATGATGGGGGAAGCTGGGCGGCAAG 3′ (SEQ ID NO: 20) is complimentary to the coding sequence 1566-1584 of Dll4. The primer also contains 18 nucleotides that encode for 6 histidines (underlined) followed by a stop codon and 6 additional bases that form a recognition site for the BamHI restriction enzyme (in bold). The recognition site is protected from degeneration by 3 additional nucleotides. The obtained PCR fragment is 358 bp in length and is digested with ClaI and BamHI restriction enzymes resulting in a product of 162 bp. The original H4-2 clone (pIpspAdapt6-Dll4) is also digested with ClaI and BamHI to release a 1411 bp fragment which is exchanged for the purified 162 bp fragment derived from the PCR experiment. The sequence of this plasmid is verified by sequencing.

This adapter vector is used to make adenovirus according to the procedure described in WO 9964582.

Soluble Dll4 Protein: Sol-Dll4-His6

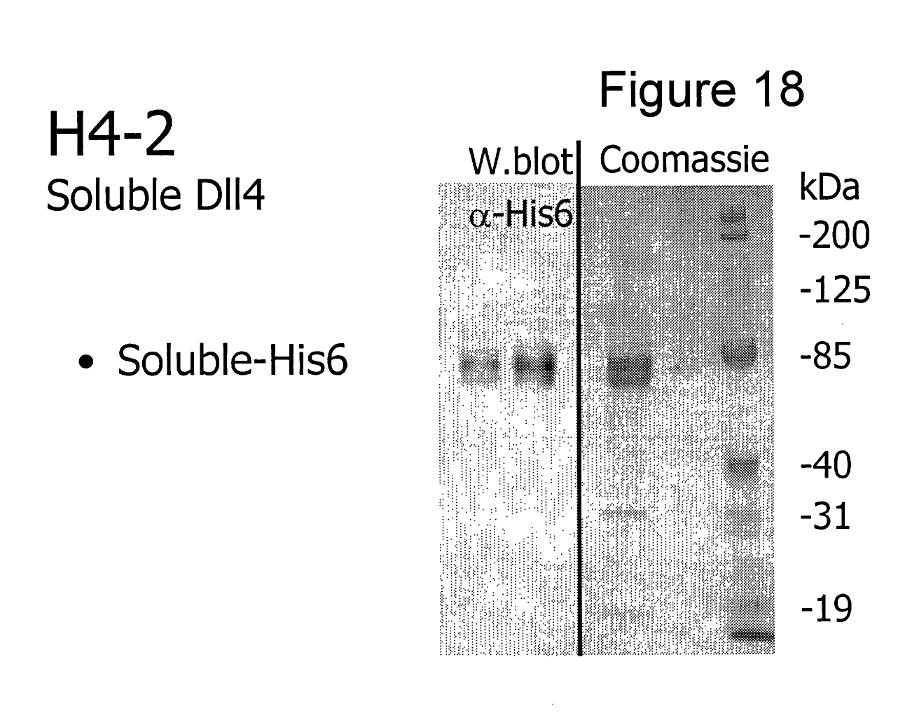

The mutant protein is produced in Per.C6 cells after adenoviral transduction. Conditioned medium is harvested and the mutant protein is purified using Ni²⁺-NTA affinity chromatography. Analysis of the purified molecule reveals only minor impurities. Western blotting using anti-His6 monoclonal antibodies reveals a His6-containing protein band that migrates with an estimated apparent molecular weight of 65-70 kDa (FIG. 18, “W. blot” panel).

Sol-Dll4-His6 Upregulates AP Activity

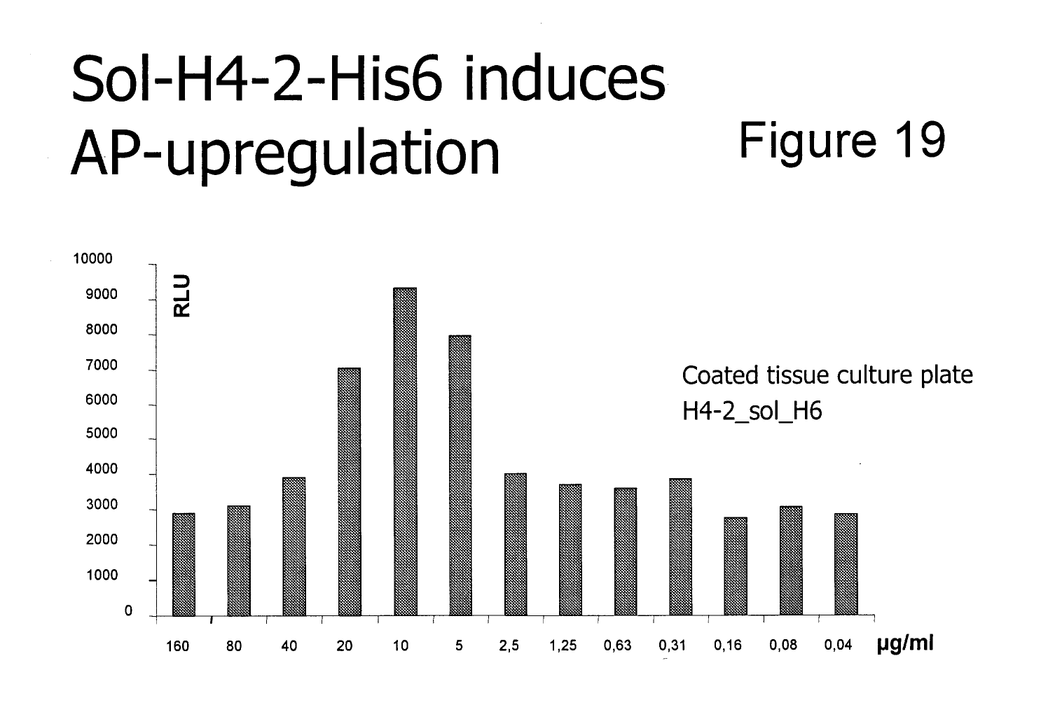

Wells of 384 microtiter plates (Costar; black walls, clear bottom) are coated with purified Sol-Dll4-His6 at various protein concentrations (15 μl of Sol-Dll4-His6 solution per well at concentrations ranging from 160 to 0.04 μg/ml). One day later, MPCs are seeded at a density of 1000 cells per well. 6 days after seeding the cells, AP activity in the wells is measured with MUP(FIG. 19). Maximal upregulation of AP activity is observed when plates are coated with 10 μg/ml of Sol-Dll4-His6.

Mineralization induced by Sol-Dll4-His6

Bone mineralization can be visualized in vitro by staining differentiated bone cells for the matrix they deposited as is discussed in example 2. Here, instead of using virus containing the full-length Dll4 ligand, 6 well plates are coated with purified Sol-Dll4-His6 and one day later, human MPCs are seeded in the coated wells at a density of 100,000 cells per well. The monolayers are stained for mineralization 21 days later. The experiment is performed as described for H4-2 and the NICDs.

### Example 13

### Identification of an Osteogenic Notch Receptor with High Affinity for Dll4

Dll4 belongs to the delta class of Notch ligands. In order to determine to which receptor Dll4 binds and which Notch receptor transduces Dll4's osteogenic signal, the following experiments are performed:

Dll4 and Notch Signaling

Full-length Notch receptors 1, 2, 3 and 4 are tagged at the C-terminus with a Gal4-VP16 fusion. Upon ligand binding to the Notch receptor, the notch intracellular domain (NICD), liberated by a proteolytic cleavage event, travels to the nucleus. The Gal4-VP16 tag allows one to study the nuclear translocation and transcriptional activity of the exogenous Notch when using an introduced reporter system with Gal4 binding sites concatamerized upstream of luciferase. The signal generated by the exogenous Notch receptor can thus be measured in the presence of the endogenous Notch receptors which cannot generate a signal in the experimental set-up used here. In addition, the nuclear translocation can be measured (e.g., by immunofluorescence) and the exogenously expressed receptor can be quantified by Western blotting.

In order to study Dll4-Notch-mediated signalling, cells are transduced with vectors containing full-length Dll4 construct, tagged at the C-terminus with the His6 tag, with vectors containing one of the 4 Notch receptors fused to the Gal4-VP16 tag and with vectors containing the Gal4-luciferase reporter construct. Dll4-Notch signalling is measured by reading luciferase-generated luminescent signals. The luminescence data are normalized for amounts of protein. In addition, Dll4-induced luciferase activity through each of the 4 Notch receptors is normalized for the amounts of Gal4-VP16 and His6 tag by Western blotting. In this way, data obtained from the 4 different experiments is compared and normalized for efficiency of transduction of both the Dll4 construct and the Notch construct.

Binding of Dll4 to Notch Receptors

Suspension cells are transduced with one of the 4 tagged Notch receptor fusion constructs and with the Gal4-luciferase reporter construct. These cells are incubated with Sol-Dll4-His6. Binding of Dll4 to the Notch receptors is measured with both the luciferase reporter and by FACS analysis. After incubation with the Sol-Dll4-His6 cells are incubated with an antibody directed against the tag. The fluorescently labelled antibody facillitates the analysis of the binding of Dll4 to cell-surface Notch receptor by submitting the cells to FACS analysis. Binding of the antibody to the cells, with a signal significantly higher than negative controls, indicates that Dll4 binds to the transduced receptor. Normalization of the data is as described above, using the luciferase reporter and Western blotting to quantitate the tagged Dll4 and Notch proteins.

Alternatively, Notch receptor binding to Dll4 can be measured by an siRNA approach. Each endogenous Notch receptor on the MPCs is knocked-down separately by siRNA technology. Dll4 is added to the cells and AP activity is determined. A decreased or abolished AP activity demonstrates that Dll4 exerts an osteogenic effect through the particular notch receptor that has been knocked-down.

Experimental:

MPC cells are seeded in a 24 well plate. The next day, siRNA constructs against one of the Notch receptors is introduced into the cell. One day later, the cell is infected by an adenovirus carrying the Dll4 cDNA. AP activity is measured 6 days later. In duplicate wells, total RNA is harvested and used for real-time PCR. Primer sets specific for the relevant Notch receptor and for BAP are used to analyze the down-regulation of the specific receptor and the Dll4-mediated up-regulation of BAP mRNA levels.

Affinity of Dll4-Notch Binding

The affinity of the Dll4-Notch binding is measured with surface plasmon resonance (Biacore). Biacore measures binding events on the surface of a sensor chip, so that the interactant attached to the surface determines the specificity of the analysis. Testing the specificity of an interaction involves simply asking whether different molecules can bind to the immobilized interactant. Binding gives an immediate change in the SPR signal, so that it is directly apparent whether an interaction takes place or not.

As sample is passed over the sensor surface, the progress of binding directly reflects the rate at which the interaction occurs. Injection of sample is followed by buffer flow during which the response reflects the rate of dissociation of the complex on the surface. Kinetic rate constants for the binding and dissociation are obtained by fitting the results to mathematical descriptions of interaction models.

Binding affinities are obtained either from rate constant measurements (the dissociation constant KD is the ratio of the rate constants kd/ka for a 1:1 interaction) or by measuring the steady state level of binding as a function of sample concentration.

The extracellular domains of both Dll4 and Notch receptors are cloned and fused in frame to an epitope tag. The tags are used to purify the extracellular domains after expression in human cells e.g., Per.C6 cells. The purified proteins are then subjected to surface plasmon resonance (SPR) (Biacore) analysis to study the affinity of Dll4 to each of the Notch receptors.

Determination of the Osteogenic Character of Notch Receptors by Tissue Profiling

Notch receptors are only physiologically relevant for bone formation when expressed in the appropriate tissue at the appropriate time. Therefore, tissue profiling of Notch receptor expression is performed on human and murine samples at different times during development. As murine samples are more available for developmental studies, especially during the embryonic stages of development, the study is mostly performed on murine samples. The study is capable of being duplicated on human tissues also.

Bony tissues (bone and/or cartilage) from mice are collected at 3 stages during embryonic development (days 11, 16 and 21 post partum) and at 1, 4 and 20 weeks postnatal. The tissues are then analyzed by immunohistochemistry, real-time PCR or in situ hybridization for the expression of Notch receptors.

Development of Agonistic Antibodies Against Osteogenic Notch Receptors

Agonistic antibodies of Notch-1 antibodies induce apoptosis in tumour cells (WO0020576A2). Notch antibodies are also described in U.S. Pat. No. 6,090,922 and induce Notch mediated signal transduction after binding to a Notch receptor. They have some advantages over the natural Notch ligands: an antibody is stable in circulation, i.e., its half-life is typically much longer than that of a receptor ligand. It is also expected to be less immunogenic than a synthetic derivative of a naturally occurring ligand. Moreover, by using bispecific antibodies, they can be guided to a specific tissue or cell type, in this case to bony tissues.

Agonistic antibodies are identified by incubating single chain Fv antibody libraries with the Notch receptor's extracellular domain as antigen. The identified antibody fragments are tested for their ability to induce up-regulation of alkaline phosphatase activity when incubated with MPCs. The antibodies that score in this screen are further subjected to a mineralization study: MPCs are seeded in 6 well plates and incubated over a period of 3-4 weeks with the antibody fragments. All antibodies that induce mineralization can then serve as substitute for the natural Notch ligand(s).

### Example 14

### Construction of Human Dll4 Mutants.

A010813-Dll4FC v1

In addition to generating p6Dll4_His, a Dll4 fusion with immunoglobulin heavy constant-fragment (Fc) is also generated.

This has several advantages:

i. The Fc portion can prolong the half-life of fusion proteins in solution,

ii. The Fc portion can aid in the purification of fusion proteins using protein A sepharose

iii. The Fc portion of the chimera induces dimerization which can be advantageous for binding of Dll4 to its receptor

A fusion protein consisting of the Dll4 soluble protein form, fused to the immunoglobulin heavy constant gamma 3-fragment (BC_{—}025314) (Fc-fragment) is constructed. The addition of a C-terminal His6 tag to the Dll4-fc-fusion protein, allows one to purify the fusion protein using Ni²⁺-NTA affinity chromatography.

The p6Dll4_ST is a construct of a soluble Dll4 as described above except that no Histidine tag is added. This construct is used to make the Fc construct: The human Dll4 sequences from the p6Dll4_ST plasmid is used as source DNA.

To obtain a fusion protein, the stop codon of the N-terminal coding sequence is removed by PCR (similar as described above). The reverse primer 5′ GGGGGATCCGGGGAAGCTGGGCGGCAAG 3′ (SEQ ID NO: 21) anneals specifically from 1566-1584 followed by a 6 additional bases that form a recognition site for the BamHI restriction enzyme. This recognition site is protected from degeneration by 3 additional nucleotides. The obtained PCR fragment is digested with ClaI and BamHI restriction enzymes producing a fragment of 149 bp. The p6Dll4_ST plasmid is digested using ClaI and BamHI, releasing a 145 bp fragment which is exchanged for the purified 149 bp fragment obtained by PCR. The resulting plasmid is prepared for the insertion of the Fc fragment by digestion with XbaI followed by a klenow treatment to blunt the ends and a subsequent digestion, after purification, with BamHI.

The immunoglobulin heavy constant gamma 3-fragment (Fc-fragment) is obtained by PCR on cDNA derived from cells isolated from peripheral blood. The forward primer 5′ CCCGGATCCCCCAAATCTTGTGACAAAACTCAC 3′ (SEQ ID NO: 22) contains 6 additional bases on the 5′ end that form a recognition site for the BamHI restriction enzyme. This recognition site is protected from degeneration by 3 additional nucleotides. The reverse primer 5′ CGCTCTGAGCTAATGGTGATGGTGATGATGTTTACCCGGAGACAGGGAGAGGCTC 3′ (SEQ ID NO: 23) has a modular design: it anneals specifically at 23 nucleotides, followed by 18 nucleotides that codes for 6 histidines, followed by a stop codon, and 9 additional nucleotides. The obtained PCR product is 731 bp in length and is digested with BamHI restriction enzyme resulting in a product of 727 bp.

This PCR product is cloned into the prepared p6Dll4_ST plasmid resulting in the final A010813-Dll4FC_v1. The A010813-Dll4FC_v1 sequence is verified by sequencing and used for adenovirus production.

### Example 15

### NICD as Inducers of Osteoblast Differentiation

NICDs are inducers of osteoblast differentiation using the osteoblast differentiation assay.

Human Notch (drosophila) homologue (NOTCH) family consist of 4 members: NOTCH-1, NOTCH-2, NOTCH-3, NOTCH-4. The notch proteins are transmembrane proteins with extracellular domains of EGF and Lin-12/Notch. These proteins are generally involved in lateral inhibition in developmental processes.

The intracellular domains of these proteins (NICD-1, NICD-2, NICD-3, NICD-4) contain Ankyrin repeats, a PEST-containing region and a nuclear localization signal region (Blaumueller et al. (1997) Cell 90, 281-291). NICDs are thought to be involved in protein-protein interactions.

Adenoviral vectors coding for the NICDs are screened using the alkaline phosphatase assay.

NICD-2

The NICD-2 cDNA is isolated using a PCR methodology. The following NICD-2-specific primers are used:

NICD-2 forward 5′-CCGGAATTCCATGGCAAAACGAAAGCGTAAGCATG-3′ (SEQ ID NO: 24)

NICD-2 reverse 5′-CGCGGATCCGTCTCTACACTGGAGGTGGACTC-3′ (SEQ ID NO: 25).

The forward primer contains a EcoRI site, the reverse primer a BamHI site.

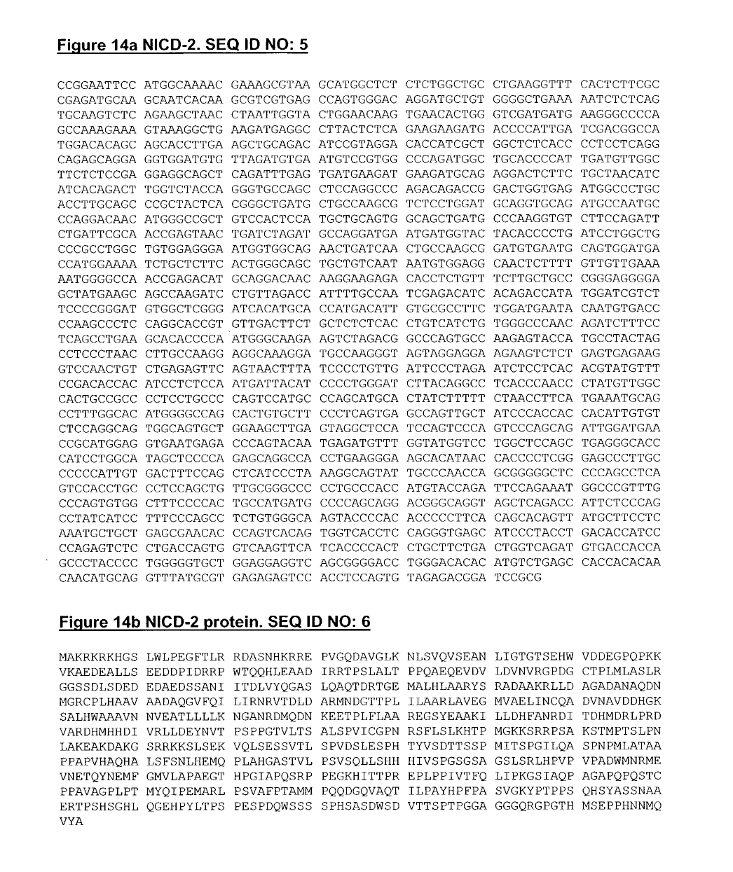

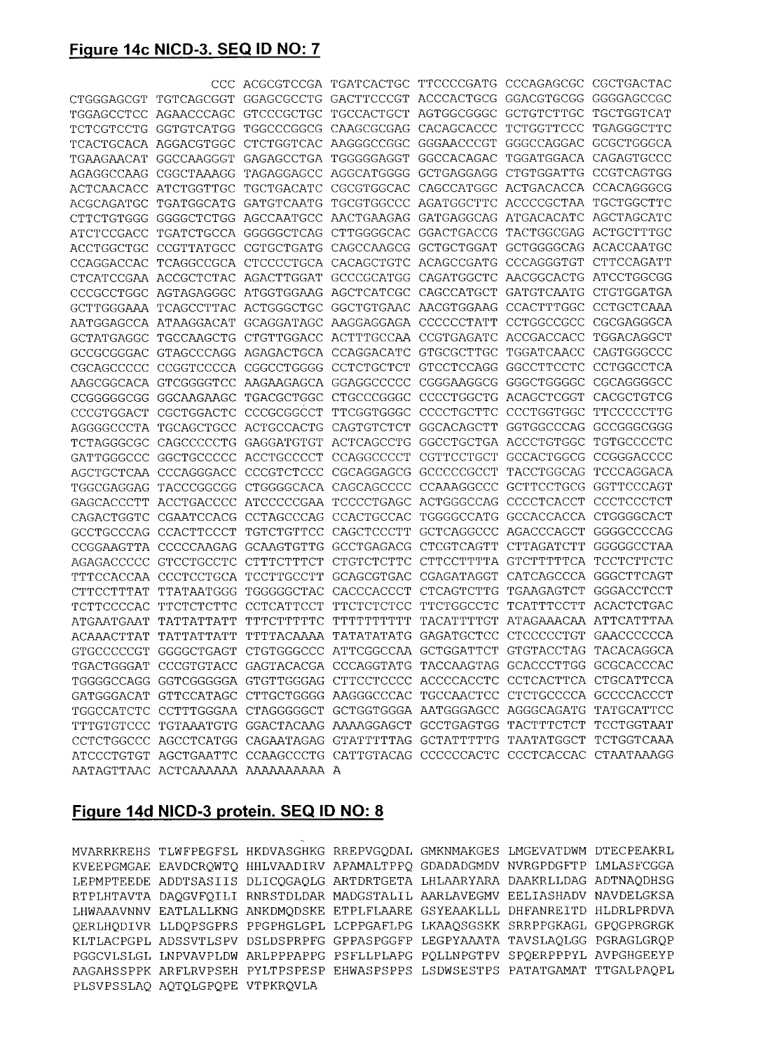

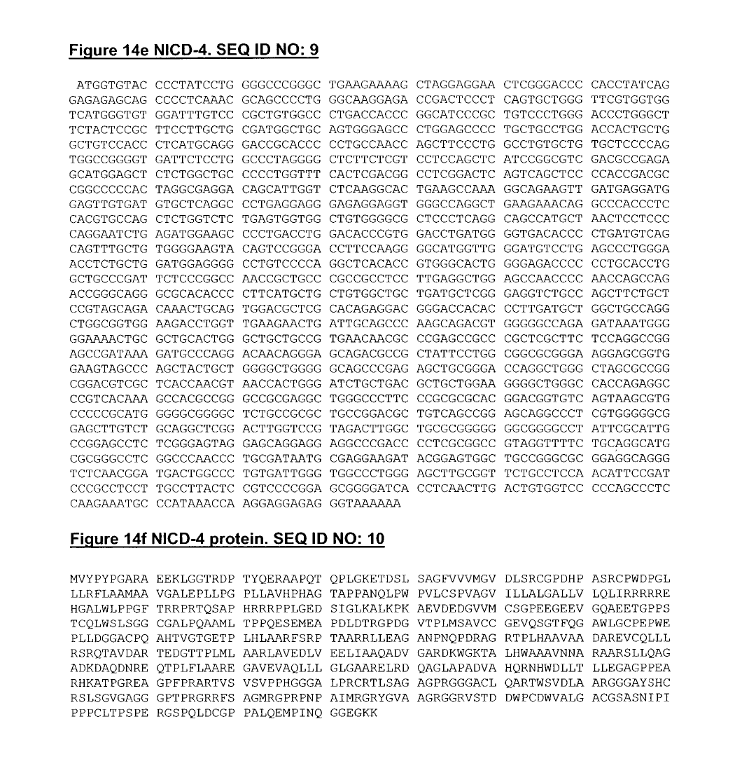

The NICD-2 cDNA is PCR amplified from placenta cDNA library but other cDNA libraries can also be used. A single fragment is obtained and digested with the HindIII and BamHI restriction enzymes. pIPspAdApt6 vector (U.S. Pat. No. 6,340,595) is digested with the same enzymes, gel-purified, and used to ligate to the digested NICD-2 PCR fragment. The sequences are depicted in FIGS. 14a and 14b as SEQ ID NO: 5 and SEQ ID NO: 6

NICD-3

The NICD-3 virus is isolated from the Placenta PhenoSelect library. Sequence determination of the cDNA insert, present in the pAdapt plasmid, shows that it is nearly identical to the one described in GenBank record NM_{—}000435, except that the cDNA is truncated at the 5′ end (about 4800 nt are missing) At the protein level, the sequence is identical to the 659 amino acids carboxy terminal half of GenBank record NP_{—}000426. The sequences are depicted in FIGS. 14c and 14d as SEQ ID NO: 7 and SEQ ID NO: 8.

NICD-4

The NICD-4 virus is isolated from the Placenta PhenoSelect library. Sequence determination of the cDNA insert, present in the pAdapt plasmid, shows that it is nearly identical to one described in GenBank record NM_{—}004557 human Notch (drosophila) homologue 4. Only the sequences coding for the intercellular domain human Notch (drosophila) homologue 4 is present in NICD-4. On the protein level, the sequence is identical to the 477 amino acids carboxy terminal half of GenBank record NM_{—}004557. The sequences are depicted in FIGS. 14e and 14f as SEQ ID NO: 9 and SEQ ID NO: 10.

The finding as disclosed in the present invention, that NICD-2, NICD-3 and NICD-4 are involved in bone homeostasis, and in particular induce osteoblast differentiation, provides new and unexpected insights in the function of this sequences, and the proteins encoded by them.

### Example 16

### NICDs as Inducers of Osteoblast Differentiation

NICDs are further characterised. The cDNAs encodes the full-length NICDs protein. NICDs is presumed to be a Notch ligand, as it is a member of the Delta family of Notch ligands. NICDs harbors the DSL and EGF-repeat domains in its extracellular region, characteristic of Notch ligands (see FIG. 15). Notch ligands induce Notch-intracellular-domain (NICD) mediated signal transduction.

Mineralization

The process of osteogenesis consists of several successive events. During the initial phases of osteogenesis, bone alkaline phosphatase (BAP) becomes up regulated. As there are 4 alkaline phosphatases and only one is bone specific, alkaline phosphatase activity per se is not a fail-safe osteogenic marker. It is therefore important to look at more specific events occurring in later stages of osteogenesis such as mineralization.

Bone tissue consists of cells embedded in a matrix of organic materials (e.g., collagen) and inorganic materials (Ca and phosphate). Bone mineralization is shown in vitro by staining differentiated bone cells for the matrix they deposited. The Von Kossa and Alizrin RedS stains allow the visualization of deposited phosphate and calcium, respectively.

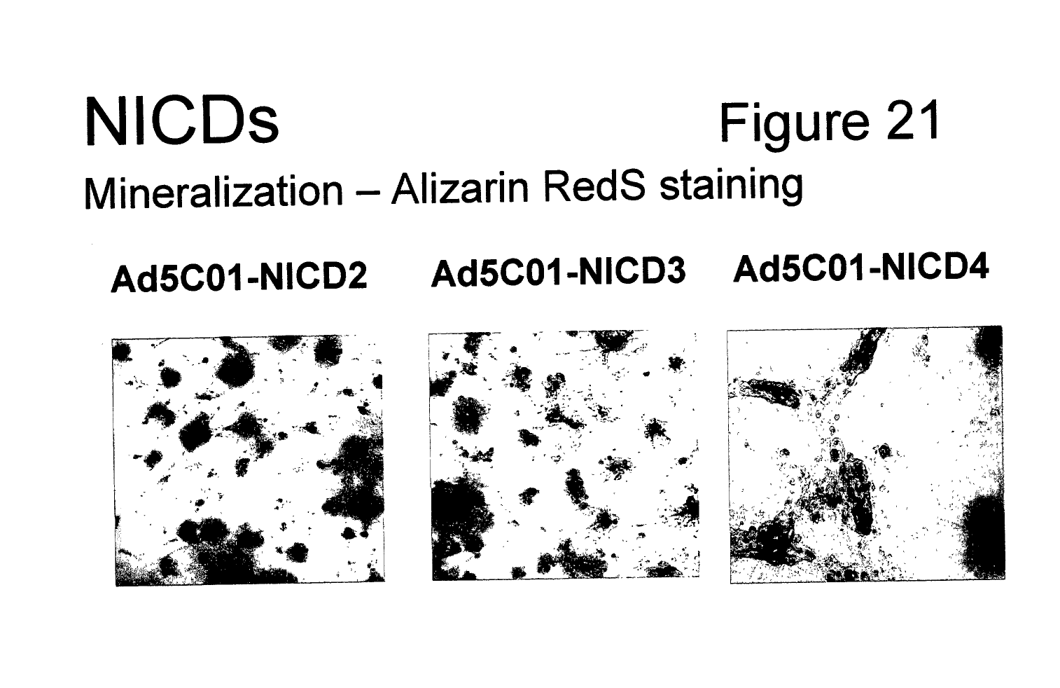

Primary human MPCs are seeded in a 6 well plate (Costar or Nunc) at a density of 50,000 cells per well and co-infected one day later with Ad5C15-hCAR (at an MOI 1000) and Ad5C01-control or hit virus (at an MOI of 500, 1500, or 4500). Medium, supplemented with 100 μg/ml L-ascorbate and 10 mM beta-glycerophosphate, is refreshed 3 times a week. 20 to 30 days after the start of the experiment, cells are stained with Alizarin RedS stain. For the Alizarin RedS staining cells are washed twice with PBS then fixed with 10% paraformaldehyde for 45 minutes at 4° C. and washed again 3 times with PBS. Cells are incubated with 40 mM aqueous Alizarin RedS solution, pH 4.1-4.3 for 10 minutes followed by 5 washes with distilled water. The staining is evaluated and photographed using white light microscopy (FIG. 21).

### Example 17

### Human FAb Phage Display to Select Human Antibodies against Validated Hits Coming Out of the BAP High-throughput Screen

In order to isolate antibodies that bind to the proteins identified herein, (characterized by SEQ ID NOS: 2, 4, 6, 8, or 10), phages displaying human FAb fragments are employed encompassing the light and heavy variable and constant regions. A human FAb phage display library is constructed in a phage display vector such as pCES1,a derivative of the pCANTAB6 vector (McGuiness et al., 1996). The library is constructed in the filamentous E. coil phage m13 and the FAb sequences are displayed as N-terminal fusion proteins with the minor coat protein pIII. The library can have a complexity of more or less than 10¹⁰ different sequences.

Three types of targets are used to select for polypeptide-displaying phages which bind to the sequences of SEQ ID NOS: 2, 4, 6, 8, or 10. Firstly, a predicted extracellular or otherwise accessible domain of SEQ ID NOS: 2, 4, 6, 8, or 10 is synthesized as a synthetic peptide. The N-terminus of this peptide is biotinylated and is followed by a KRR peptide linker and the predicted sequence of SEQ ID NOS: 2, 4, 6, 8, or 10, respectively.

Secondly, a fusion protein is made of a portion or of the complete protein of the invention (characterized by a SEQ ID NO: 2, 4, 6, 8, or 10) in frame with the ORF of either a lutathione-S-transferase (GST), a maltose-binding protein, a His6 tag or any other tag. The resulting protein is expressed in E. coli. Equally well, a His6 or another tag is fused in frame with the ORF of the sequence and expressed in a mammalian expression system such as PER.C6/E2A. Fusion proteins are then purified using for example Ni²⁺-NTA columns for His6-tagged proteins (Qiagen) or glutathione resin (Pharmacia) for GST-tagged proteins.

Thirdly, for example membrane-bound polypeptides, cell lines derived from the human HeLa cell line are generated that overexpress SEQ ID NO: 2 Ad5C01_Hdelta2 (H4-2). Overexpression can be by steady state mRNA expression levels, or, in case the protein is tagged by fusing an epitope tag in frame to the ORF of SEQ ID NO: 2, by immunological detection of the epitope-tagged protein.

To select for FAb displaying phages that bind to SEQ ID NO: 2, 4, 6, 8, or 10 the following selection procedure is employed. A pool of Fab displaying phage is selected out of a complex mixture of a high number of different Fab displaying phages in four rounds by their ability to bind with significant affinity to a biotinylated peptide or to a purified fusion protein that has been expressed in E coli or in a mammalian expression system such as PER.C6/E2A. The collection of selected Fab displaying phage is further decreased by the next selection procedure: the Fab displaying phage are further selected in three rounds for their ability to bind to SEQ ID NO: 2, 4, 6, 8, or 10 present in cell lysates from cells overexpressing SEQ ID NO: 2. For selection on biotinylated peptide 250 μl of FAb library (or eluted phage from the previous round) is mixed with 250 μl 4% Marvel in PBS and equilibrated while rotating at RT for 1 hour. Subsequently biotinylated hCAT1 peptide (20-500 nM in H₂O) is added. This mix is incubated on the rotator at RT for 1 hour before 250 ml equilibrated streptavidin-dynabeads in 2% Marvel in PBS is added. After incubation on a rotator at RT for 15 min the beads with the bound phage are washed 5 times with PBS/2% Marvel/0.1% Tween, 5 times with PBS /0.1% Tween and 5 times with PBS. Then the phage are eluted by incubation with 0.1M Tri-ethyl-amine on a rotator at RT for 10 min and neutralized in 1 M Tris-HCl pH 7.4.

The eluted phages are titered and amplified in TG1 before the next selection.

For selection on plasma membrane-bound SEQ ID NO: 2 (H4-2) expressing cells, the cells are harvested at a confluence of about 80% and suspended in PBS/10% FBS/2% Marvel to a final concentration of at least 3×10⁶ cells/ml. This cell suspension is incubated for 30 min on a rowing boat mixer (or rotator), while at the same time phage are also preincubated in PBS/10% FBS/2% Marvel. Then the cells are centrifuged, resuspended in the preincubated phage solution and incubated on a rowing boat mixer (or rotator) for 1 hour. Afterwards the cells are washed 5-10 times with PBS/10% FBS/2% Marvel and twice with PBS. The cells are centrifuged and resuspended in 0.6 ml water. Subsequently 0.6 ml 200 mM triethylamine is added (drop wise while vortexing). After 5 minutes the suspension is neutralized by adding 0.6 ml of 1 M Tris-HCl pH 7.4 (drop wise while vortexing).After centrifugation (5 min, 14000 rpm) the supernatant is transferred to a new tube and titered and amplified in TG1 before the next selection or final cloning step.

The pools of the last various selection rounds are tested for binding to the biotinylated peptides or preferably the fusion or purified full length proteins in a specific ELISA and also for cell binding by flow cytometric analysis where appropriate. Once FAB displaying and SEQ ID NO: 2 antigen binding clones are isolated, double strand phagemid DNA is prepared and used to determine the nucleotide and deduced amino acid sequence of the displayed variable heavy and light chains.

The Fab phages or antibodies derived thereof can be used as diagnostic tools, for example in immunohistochemistry, as research tools, for example in affinity chromatography, as therapeutic antibodies directly, or for the generation of therapeutic antibodies by generating anti-idiotypic antibodies.

### Example 18

### Generation of Soluble hDelta2 (H4-2) and Rescreening It in the BAP High-throughput Screening Assay

hDelta2 (SEQ ID NO: 2) is a transmembrane ligand for receptors of the Notch family. A soluble ligand derived from SEQ ID NO: 2 (SEQ ID NO: 4) is easier to convert into a biopharmaceutical than a membrane-bound ligand. Moreover, it is often seen that membrane-bound ligands, such as TNF-alpha, are shed into the extracellular milieu retaining their activity as a ligand. A soluble Delta2 ligand can be used as a drug for the treatment of osteoporosis and bone diseases as described above.

In order to study the activity of soluble Delta2 as an inducer of osteoblast differentiation, a His6-epitope tag is fused in frame to SEQ ID NO: 4, which is a part of SEQ ID NO: 2. In addition, a soluble version without a His6 tag is made. Delta2 contains two hydrophobic stretches, representing transmembrane domains: the N-terminal amino acids that make up the signal peptide and the residues in the C-terminal region comprising most probably the amino acids 529 to 552. The latter hydrophobic stretch probably serves as the transmembrane region anchoring the ligand into cellular membranes, mostly the plasma membrane. By using molecular biology techniques known in the art, this hydrophobic domain and the amino acids downstream from this domain are removed. This is achieved by amplifying part of the molecule using the primer set

| | | |
|---|---|---|
| 5′-AACCGAGGTCCAAGCCGCATGTGCC | (forward) | (SEQ ID NO:19) |
| and | | |
| 5′-CGCGGATCCTCAGGGGAAGCTGGGCGGCAAG | (reverse) | (SEQ ID NO:26) |
| or the primer set | | |
| 5′-AACCGAGGTCCAAGCCGCATGTGCC | (forward) | (SEQ ID NO:19) |
| and | | |
| 5′-CGCGGATCCTAATGGTGATGGTGATGATGGGGGAAGCTGGGCGGCAAG. | (reverse) | (SEQ ID NO:20) |

PCR-amplified double stranded DNA products are digested using restriction enzyme ClaI and subcloned in the parental plasmid, which is used as PCR-template. The first primer set (SEQ ID NOS: 19 and 26) allows the generation of an untagged soluble version of SEQ ID NO: 4, the second primer set (SEQ ID NOS: 19 and 20) allows the generation of a soluble C-terminally tagged His6 fusion protein of SEQ ID NO: 4. Adenoviruses are generated and propagated starting from this plasmid essentially as described under Example 1. These are then screened in the BAP screen l for upregulation of endogenous BAP activity. The soluble versions of SEQ ID NO: 2 (SEQ ID NO: 4 and His-tagged SEQ ID NO: 4) are then compared to the activity of wild-type SEQ ID NO: 2 and to negative and positive control viruses (BMP2, BMP7, FosB, PLAP, eGFP, LacZ and luciferase).

Moreover, the activity of the aforementioned viruses is compared in a supernatant transfer experiment, as described in detail under Examples 10, 4 and 5 . The BAP inducing supernatants containing the soluble forms of SEQ ID NO: 2 are also tested for induction of other differentiation specific genes including for example BAP, BMP2, BMP7, osteocalcein, osteopontin, and for the potential to induce bone formation in vitro as well as in vivo.

### Example 19

### In vivo Validation of Hits from the High-throughput BAP-screen

In vivo, the osteoblast-induction potential is tested in a number of ways. Both down-regulation and overexpression of SEQ ID NOS: 1, 2, 3, 4, 5, 6, 7, 8, 9, and 10 are tested.

For down-regulating expression of SEQ ID NOS: 1, 2, 3, 4, 5, 6, 7, 8, 9, and 10, knock-out animals, preferably mice, are generated according to established procedures. In short, one or more exons of the genes encoding SEQ ID NO: 2, 6, 8, or 10 are deleted by homologous recombination in mouse ES cells. These ES cells have been isolated from a limited number of homozygous strains of inbred lab mice well-suited to derive knock-out mice and are well known for those skilled in the art. Removal of one or more exons is checked by techniques such as southern blotting and the diploid state of ES cells is checked by cytogenetic techniques. Knock-out ES cells harboring the expected microdeletion and the expected number of chromosomes are then used to derive mice, according to established procedures. Resulting chimeric mice are then used to start a colony of knock-out mice where the mice can be hetero- or homozygous for the allele in which one or more exons of the gene corresponding to SEQ ID NOS: 1, 2, 5, 6, 7, 8, 9, and 10 are deleted. Both homo- and heterozygous knock-out mice are then used to study the bone homeostasis occurring in these mice, in comparison with wild-type mice, i.e., mice from the same inbred homozygous strain that have the gene corresponding to SEQ ID NOS: 1, 2, 5, 6, 7, 8, 9, and 10 intact. The absence of expression of said SEQ ID NOS: is studied by western blotting and northern blotting, performed on tissues, including bone tissue of wild-type and knock-out animals. The effect of the absence of expression of SEQ ID NOS: 1, 2, 5, 6, 7, 8, 9, and 10 is studied on bone biology in a number of ways: we analyze physical parameters such as the presence of all bones, normally seen in healthy wild-type animals, bone thickness, length of bones. Furthermore, the bones are examined in more detail using histochemical techniques where, for example, the extent of mineralization and numbers of osteoblasts are examined and, combined with the use of specific antibodies (immunohistochemistry), the presence and subcellular localization of osteoblast-specific proteins is examined.

For overexpressing SEQ ID NOS: 1, 2, 3, 4, 5, 6, 7, 8, 9, and 10 in vivo, preferably in mice, the following procedure is used: SEQ ID NOS: 1, 3, 5, 7, and 9 are subcloned into a eukaryotic expression plasmid, downstream of a ubiquitously expressed promoter or, preferably, downstream of a promoter allowing for expression only in the bone compartment. The plasmid containing the above-mentioned promoter and SEQ ID NOS: 1, 3, 5, 7, and 9 is then used to derive transgenic mice according to established procedures. Homozygous mouse strains, well-suited to derive transgenic mice, such as the FVB strain are used. Exogenous expression of SEQ ID NOS: 1, 2, 3, 4, 5, 6, 7, 8, 9, and 10 is analyzed using southern blot, allowing an estimation of the copy number of the expression cassette, integrated in the mouse genome and also by northern or western blotting, if antibodies are available. The effect of the exogenous expression of SEQ ID NOS: 1, 2, 3, 4, 5, 6, 7, 8, 9, and 10 on bone biology is analyzed as described above for knock-out animals.

[image]

[image]

[image]

[image]

[image]

[image]

[image]

We claim:

1. An isolated nucleic acid selected from a group of nucleic acids identifiable as inducers of osteoblast differentiation consisting of:
(a) a DNA sequence of SEQ ID NO: 1, 3, 5, 7, or 9 or the complement thereof,
(b) a RNA sequence corresponding to SEQ ID NO: 1, 3, 5, 7, or 9 or the complement thereof,
(c) a nucleic acid specifically hybridizing to one of the nucleotide sequences defined in (a) or (b),
(d) a nucleic acid encoding a protein comprising the amino acid sequence of SEQ ID NO: 2, 4, 6, 8, or 10,
(e) a nucleic acid encoding an immunologically active and/or functional fragment of a protein encoded by a DNA sequence of SEQ ID NO: 1, 3, 5, 7, or 9,
(f) a nucleic acid encoding a protein as defined in SEQ ID NO: 2, 4, 6, 8, or 10, or a nucleic acid as defined in any one of (a) to (e) wherein said sequence is bNA, cDNA, genomic DNA or synthetic DNA.

2. A vector comprising a nucleic acid sequence according to claim 1.

3. The vector of claim 2, wherein said vector is an expression vector wherein the nucleic acid sequence is operably linked to one or more control sequences allowing the expression of said sequence in prokaryotic and/or eukaryotic host cells.

4. The vector of claim 3, wherein said vector is an adenoviral vector.

5. The vector of claim 4, wherein said vector is generated from an adenoviral adapter vector which contains the left ITR and part of the E2B region, and in which the E1 region has been exchanged for a mammalian promoter, a polylinker, and a polyadenylation signal.

6. A polynucleotide sequence comprising a nucleic acid according to claim 1, in which said nucleic acid encodes an antisense nucleic acid.

7. A vector comprising a polynucleotide sequence according to claim 6.

8. The vector according to claim 7, wherein said vector is an expression vector wherein the antisense polynucleotide sequence is operably linked to one or more control sequences allowing the expression of said sequence in prokaryotic and/or eukaryotic host cells.

9. A method for regulating gene expression comprising:
(a) introducing a polynucleotide sequence comprising a nucleic acid sequence according to claim 1 into a host cell,
(b) regulating the expression of a target gene, wherein said polynucleotide sequence regulates the transcription or expression of said target gene.

10. A method for modulating osteoblast differentiation comprising introducing a polynucleotide sequence comprising a nucleic acid sequence according to claim 1 into a host cell, under conditions resulting in the expression of said polynucleotide sequence in said host cell.

11. A host cell comprising an integrated or episomal copy of a nucleic acid molecule of claim 1 or 6.

12. The host cell of claim 11, wherein said host cell is a yeast, bacterial, insect, fungal, plant or mammalian cell.

13. An isolated polypeptide encoded by a nucleic acid of claim 1, or a variant or a derivative thereof, or an immunologically active and/or functional fragment thereof.

14. A polypeptide having an amino acid sequence of SEQ ID NO: 2, 4, 6, 8, or 10, or a variant or a derivative thereof, or an immunologically active and/or functional fragment thereof.

15. A method for producing a polypeptide according to claim 13, comprising:
(a) culturing host cells comprising a nucleic acid according to claim 1, under conditions allowing the expression of the polypeptide, and,
(b) recovering said polypeptide from said culture.

16. A method for producing an antibody specifically recognizing a polypeptide encoded by a nucleic acid according to claim 1, or a specific epitope of said polypeptide.

17. A method for detecting a nucleic acid according to claim 1 or a polypeptide according to claim 13.

18. An isolated recombinant nucleic acid according to claim 1 for use as a medicament.

19. A pharmaceutical composition comprising a substantially purified recombinant nucleic acid according to claim 1 in a pharmaceutically effective amount for preventing, treating and/or alleviating diseases involving the absence or decreased induction of osteoblast differentiation, in combination with a pharmaceutically acceptable carrier.

20. A method for inducing osteoblast differentiation, comprising:
(a) introducing a nucleic acid according to claim 1 in a desired host cell capable of further cell differentiation, in vitro or in vivo,
(b) expressing said nucleic acid, and,
(c) stimulating the differentiation of said host cells into osteoblasts.

21. A method for in vitro production of bone tissue, comprising the steps of:
(a) applying undifferentiated mammalian cells on a substrate to form a cellular substrate,
(b) introducing a polynucleotide according to claim 1 into said cells, or contacting said cellular substrate with the polypeptide encoded by said polynucleotide for a time sufficient to differentiate said undifferentiated cells into osteoblasts, thereby producing a continuous bone matrix.

22. A method according to claim 21, wherein said continuous bone matrix comprises a thickness of at least 0.5 μm on the surface of said substrate.

23. A method for diagnosing a pathological condition relating to abnormal osteoblast differentiation or a susceptibility to said condition in a subject comprising:
a) obtaining a sample of said subject's mRNA corresponding to a nucleic acid of claim 1 or a sample of said subject's genomic DNA corresponding to a genomic sequence of a nucleic acid of claim 1;
b) determining the nucleic acid sequence of said subject's mRNA or genomic DNA;
c) comparing the nucleic acid sequence of said subject's mRNA or genomic DNA with a nucleic acid of claim 1 or with a genomic sequence encoding a nucleic acid of claim 1 obtained from a database; and
d) identifying any difference(s) between the nucleic acid sequence of said subject's mRNA or genomic DNA and said nucleic acid of claim 1 or said genomic sequence encoding a nucleic acid of claim 1 obtained from a database.

24. A method for diagnosing a pathological condition relating to abnormal osteoblast differentiation or a susceptibility to said condition in a subject comprising determining the presence or the amount of the nucleic acid of claim 1, or the expression of the polypeptide encoded by said nucleic acid, in a biological sample.

25. A kit for the diagnosis of abnormal osteoblast differentiation in a patient comprising a nucleic acid according to claim 1.

26. A kit for the diagnosis of abnormal osteoblast differentiation in a patient comprising a polypeptide according to claim 13.

27. A transgenic non-human animal comprising one or more copies of a nucleic acid of claim 1 stably integrated into the genome of said animal, or an animal comprising regulatory elements that modulate the expression of a nucleic acid of claim 1.

28. A knock-out non-human animal comprising a deletion of one or more exons from one or two alleles encoding a nucleic acid of claim 1, or the deletion of one or more exons of said nucleic acid, or an animal comprising a targeted mutation in the genomic region, including regulatory sequences, comprising a nucleic acid sequence of claim 1.

29. An assay for screening test compounds to identify compounds that may induce or repress osteoblast differentiation, comprising:
(a) combining said test compound with an agent consisting of the polypeptide according to claim 13, or a variant or a derivative thereof, or an immunologically active and/or functional fragment thereof, under conditions that allow said agent to contact said test compound;
(b) detecting the formation of a complex that is an indicator of the interaction of said agent and test compound.

30. A drug compound screening assay to identify polynucleotide or polypeptide test compounds that may be useful in preventing, treating and/or alleviating diseases or disorders involving abnormal osteoblast differentiation, comprising:
(a) contacting said test compound with a nucleic acid according to claim 1, and
(b) determining whether said compound interacts with said nucleic acid.

31. A drug compound screening assay for the identification of test compounds which modulate the interaction between a protein that induces osteoblast differentiation and a target molecule, comprising:
(a) combining (i) a test compound, (ii) an agent consisting of a polypeptide according to claim 13, or a variant or a derivative thereof, or an immunologically active and/or functional fragment thereof, and (iii) a target molecule to which said agent interacts in the absence of a test compound, under conditions that allow the interaction thereof, and
(b) detecting the formation of a complex that includes either said agent and the target molecule, or said agent and the test compound.

32. A drug compound screening method for the identification of test compounds that modulate the expression of a osteoblast differentiation gene comprising the nucleic acid according to claim 1, said assay comprising:
(a) providing a host cell transfected with an expression vector comprising the nucleic acid according to claim 1,
(b) introducing a test compound into said host cell, and
(c) measuring the expression levels of polypeptide encoded by said nucleic acid.

33. A drug compound screening assay for identifying compounds that modulate the interaction between binding partners in a complex, in which at least one of said binding partners is an agent consisting of a polypeptide according to claim 13, or a variant or a derivative thereof, or an immunologically active and/or functional fragment thereof, said method comprising:
(a) contacting a test compound with the complex, for a time sufficient to modulate the interaction in the complex;
(b) monitoring said complex for changes in complex interaction;
(c) identifying test compounds that alter interactions in said complex.

34. A method for producing osteoblasts for an implant comprising:
(a) isolating mesenchymal pluripotent cells (MPCs) from bone marrow, fat tissue or other autologous sources containing MPCs,
(b) inducing ex vivodifferentiation of said MPCs into osteoblasts by introducing a polynucleotide sequence according to claim 1 into said MPCs, or by contacting a polypeptide according to claim 13 with said MPCs,
(c) isolating the osteoblasts produced in step (b).

35. A method for producing an implant comprising:
(a) mixing the osteoblasts of claim 34, step (c) with a matrix or matrix-forming material to form a osteoblast composition, and
(b) applying said osteoblast composition to a synthetic graft to produce an implant.

36. A composition for the treatment of defects in bones comprising a matrix or a matrix-forming material used to fill a bone defect and a polypeptide according to claim 13, at a concentration effective to induce osteoblast differentiation.

37. A composition for the treatment of defects in bones comprising a matrix or a matrix-forming material used to fill a bone defect and an effective amount of a transfectable vector comprising an expressible polynucleotide according to claim 1.

38. A polypeptide encoded by an isolated recombinant nucleic acid according to claim 1 for use as a medicament.

39. A pharmaceutical composition comprising a polypeptide encoded by an isolated recombinant nucleic acid according to claim 1 in a pharmaceutically effective amount for preventing, treating and/or alleviating diseases involving the absence or decreased induction of osteoblast differentiation, in combination with a pharmaceutically acceptable carrier.


SEQUENCE LISTING (26 sequences)

SEQ ID NO: 1 - DNA, 3104 bp, human
gccgccttggtgcagcgtacaccggcactagcccgcttgcagccccaggattagacagaagacgcgtcctcggcgcggtcgccgcccagccgtagtcacctggattacctacagcggcagctgcagcggagccagcgagaaggccaaaggggagcagcgtcccgagaggagcgcctcttttcagggaccccgccggctggcggacgcgcgggaaagcggcgtcgcgaacagagccagattgagggcccgcgggtggagagagcgacgcccgaggggatggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatatgctcccccagggactccatgtaccagtctgtgtgtttgatatcagaggagaggaatgaatgtgtcattgccacggaggtataaggcaggagcctacctggacatccctgctcagccccgcggctggaccttccttctgcattgtttacattgcatcctggatgggacgtttttcatatgcaacgtgctgctctcaggaggaggagggaatggcaggaaccggacagactgtgaacttgccaagagatgcaatacccttccacacctttgggtgtctgtctggcatcagattggcagctgcaccaaccagaggaacagaagagaagagagatgccactgggcactgccctgccagtagtggccttcagggggctccttccggggctccggcctgttttccagagagagtggcagtagccccatggggcccggagctgctgtggcctccactggcatccgtgtttccaaaagtgcctttggcccaggctccacggcgacagttgggcccaaatcagaaaggagagagggggccaatgagggcagggcctcctgtgggctggaaaaccactgggtgcgtctcttgctggggtttgccctggaggtgaggtgagtgctcgagggaggggagtgctttctgccccatgcctccaactactgtatgcaggcctggctctctggtctaggccctttgggcaagaatgtccgtctacccggcttccaccaccctctggccctgggcttctgtaagcagacaggcagagggcctgcccctcccaccagccaagggtgccaggcctaactggggcactcagggcagtgtgttggaaattccactgagggggaaatcaggtgct

SEQ ID NO: 2 - protein, 685 aa, human
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 3 - DNA, 1584 bp, human
atggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccc

SEQ ID NO: 4 - protein, 528 aa, human
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFP

SEQ ID NO: 5 - DNA, 2366 bp, human
ccggaattccatggcaaaacgaaagcgtaagcatggctctctctggctgcctgaaggtttcactcttcgccgagatgcaagcaatcacaagcgtcgtgagccagtgggacaggatgctgtggggctgaaaaatctctcagtgcaagtctcagaagctaacctaattggtactggaacaagtgaacactgggtcgatgatgaagggccccagccaaagaaagtaaaggctgaagatgaggccttactctcagaagaagatgaccccattgatcgacggccatggacacagcagcaccttgaagctgcagacatccgtaggacaccatcgctggctctcacccctcctcaggcagagcaggaggtggatgtgttagatgtgaatgtccgtggcccagatggctgcaccccattgatgttggcttctctccgaggaggcagctcagatttgagtgatgaagatgaagatgcagaggactcttctgctaacatcatcacagacttggtctaccagggtgccagcctccaggcccagacagaccggactggtgagatggccctgcaccttgcagcccgctactcacgggctgatgctgccaagcgtctcctggatgcaggtgcagatgccaatgcccaggacaacatgggccgctgtccactccatgctgcagtggcagctgatgcccaaggtgtcttccagattctgattcgcaaccgagtaactgatctagatgccaggatgaatgatggtactacacccctgatcctggctgcccgcctggctgtggagggaatggtggcagaactgatcaactgccaagcggatgtgaatgcagtggatgaccatggaaaatctgctcttcactgggcagctgctgtcaataatgtggaggcaactcttttgttgttgaaaaatggggccaaccgagacatgcaggacaacaaggaagagacacctctgtttcttgctgcccgggaggggagctatgaagcagccaagatcctgttagaccattttgccaatcgagacatcacagaccatatggatcgtcttccccgggatgtggctcgggatcacatgcaccatgacattgtgcgccttctggatgaatacaatgtgaccccaagccctccaggcaccgtgttgacttctgctctctcacctgtcatctgtgggcccaacagatctttcctcagcctgaagcacaccccaatgggcaagaagtctagacggcccagtgccaagagtaccatgcctactagcctccctaaccttgccaaggaggcaaaggatgccaagggtagtaggaggaagaagtctctgagtgagaaggtccaactgtctgagagttcagtaactttatcccctgttgattccctagaatctcctcacacgtatgtttccgacaccacatcctctccaatgattacatcccctgggatcttacaggcctcacccaaccctatgttggccactgccgcccctcctgccccagtccatgcccagcatgcactatctttttctaaccttcatgaaatgcagcctttggcacatggggccagcactgtgcttccctcagtgagccagttgctatcccaccaccacattgtgtctccaggcagtggcagtgctggaagcttgagtaggctccatccagtcccagtcccagcagattggatgaaccgcatggaggtgaatgagacccagtacaatgagatgtttggtatggtcctggctccagctgagggcacccatcctggcatagctccccagagcaggccacctgaagggaagcacataaccacccctcgggagcccttgccccccattgtgactttccagctcatccctaaaggcagtattgcccaaccagcgggggctccccagcctcagtccacctgccctccagctgttgcgggccccctgcccaccatgtaccagattccagaaatggcccgtttgcccagtgtggctttccccactgccatgatgccccagcaggacgggcaggtagctcagaccattctcccagcctatcatcctttcccagcctctgtgggcaagtaccccacacccccttcacagcacagttatgcttcctcaaatgctgctgagcgaacacccagtcacagtggtcacctccagggtgagcatccctacctgacaccatccccagagtctcctgaccagtggtcaagttcatcaccccactctgcttctgactggtcagatgtgaccaccagccctacccctgggggtgctggaggaggtcagcggggacctgggacacacatgtctgagccaccacacaacaacatgcaggtttatgcgtgagagagtccacctccagtgtagagacggatccgcg

SEQ ID NO: 6 - protein, 773 aa, human
MAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDHMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 7 - DNA, 3234 bp, human
cccacgcgtccgatgatcactgcttccccgatgcccagagcgccgctgactacctgggagcgttgtcagcggtggagcgcctggacttcccgtacccactgcgggacgtgcggggggagccgctggagcctccagaacccagcgtcccgctgctgccactgctagtggcgggcgctgtcttgctgctggtcattctcgtcctgggtgtcatggtggcccggcgcaagcgcgagcacagcaccctctggttccctgagggcttctcactgcacaaggacgtggcctctggtcacaagggccggcgggaacccgtgggccaggacgcgctgggcatgaagaacatggccaagggtgagagcctgatgggggaggtggccacagactggatggacacagagtgcccagaggccaagcggctaaaggtagaggagccaggcatgggggctgaggaggctgtggattgccgtcagtggactcaacaccatctggttgctgctgacatccgcgtggcaccagccatggcactgacaccaccacagggcgacgcagatgctgatggcatggatgtcaatgtgcgtggcccagatggcttcaccccgctaatgctggcttccttctgtgggggggctctggagccaatgccaactgaagaggatgaggcagatgacacatcagctagcatcatctccgacctgatctgccagggggctcagcttggggcacggactgaccgtactggcgagactgctttgcacctggctgcccgttatgcccgtgctgatgcagccaagcggctgctggatgctggggcagacaccaatgcccaggaccactcaggccgcactcccctgcacacagctgtcacagccgatgcccagggtgtcttccagattctcatccgaaaccgctctacagacttggatgcccgcatggcagatggctcaacggcactgatcctggcggcccgcctggcagtagagggcatggtggaagagctcatcgccagccatgctgatgtcaatgctgtggatgagcttgggaaatcagccttacactgggctgcggctgtgaacaacgtggaagccactttggccctgctcaaaaatggagccaataaggacatgcaggatagcaaggaggagacccccctattcctggccgcccgcgagggcagctatgaggctgccaagctgctgttggaccactttgccaaccgtgagatcaccgaccacctggacaggctgccgcgggacgtagcccaggagagactgcaccaggacatcgtgcgcttgctggatcaacccagtgggccccgcagcccccccggtccccacggcctggggcctctgctctgtcctccaggggccttcctccctggcctcaaagcggcacagtcggggtccaagaagagcaggaggccccccgggaaggcggggctggggccgcaggggccccgggggcggggcaagaagctgacgctggcctgcccgggccccctggctgacagctcggtcacgctgtcgcccgtggactcgctggactccccgcggcctttcggtgggccccctgcttcccctggtggcttcccccttgaggggccctatgcagctgccactgccactgcagtgtctctggcacagcttggtggcccaggccgggcgggtctagggcgccagccccctggaggatgtgtactcagcctgggcctgctgaaccctgtggctgtgcccctcgattgggcccggctgcccccacctgcccctccaggcccctcgttcctgctgccactggcgccgggaccccagctgctcaacccagggacccccgtctccccgcaggagcggcccccgccttacctggcagtcccaggacatggcgaggagtacccggcggctggggcacacagcagccccccaaaggcccgcttcctgcgggttcccagtgagcacccttacctgaccccatcccccgaatcccctgagcactgggccagcccctcacctccctccctctcagactggtccgaatccacgcctagcccagccactgccactggggccatggccaccaccactggggcactgcctgcccagccacttcccttgtctgttcccagctcccttgctcaggcccagacccagctggggccccagccggaagttacccccaagaggcaagtgttggcctgagacgctcgtcagttcttagatcttgggggcctaaagagacccccgtcctgcctcctttctttctctgtctcttccttccttttagtctttttcatcctcttctctttccaccaaccctcctgcatccttgccttgcagcgtgaccgagataggtcatcagcccagggcttcagtcttcctttatttataatgggtgggggctaccacccaccctctcagtcttgtgaagagtctgggacctccttcttccccacttctctcttccctcattcctttctctctccttctggcctctcatttccttacactctgacatgaatgaattattattatttttctttttctttttttttttacattttgtatagaaacaaattcatttaaacaaacttattattattattttttacaaaatatatatatggagatgctccctccccctgtgaaccccccagtgcccccgtggggctgagtctgtgggcccattcggccaagctggattctgtgtacctagtacacaggcatgactgggatcccgtgtaccgagtacacgacccaggtatgtaccaagtaggcacccttgggcgcacccactggggccaggggtcgggggagtgttgggagcttcctccccaccccacctccctcacttcactgcattccagatgggacatgttccatagccttgctggggaagggcccactgccaactccctctgccccagccccacccttggccatctccctttgggaactagggggctgctggtgggaaatgggagccagggcagatgtatgcattcctttgtgtccctgtaaatgtgggactacaagaaaaggagctgcctgagtggtactttctcttcctggtaatcctctggcccagcctcatggcagaatagaggtatttttaggctatttttgtaatatggcttctggtcaaaatccctgtgtagctgaattcccaagccctgcattgtacagccccccactcccctcaccacctaataaaggaatagttaacactcaaaaaaaaaaaaaaaaa

SEQ ID NO: 8 - protein, 659 aa, human
MVARRKREHSTLWFPEGFSLHKDVASGHKGRREPVGQDALGMKNMAKGESLMGEVATDWMDTECPEAKRLKVEEPGMGAEEAVDCRQWTQHHLVAADIRVAPAMALTPPQGDADADGMDVNVRGPDGFTPLMLASFCGGALEPMPTEEDEADDTSASIISDLICQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHFANREITDHLDRLPRDVAQERLHQDIVRLLDQPSGPRSPPGPHGLGPLLCPPGAFLPGLKAAQSGSKKSRRPPGKAGLGPQGPRGRGKKLTLACPGPLADSSVTLSPVDSLDSPRPFGGPPASPGGFPLEGPYAAATATAVSLAQLGGPGRAGLGRQPPGGCVLSLGLLNPVAVPLDWARLPPPAPPGPSFLLPLAPGPQLLNPGTPVSPQERPPPYLAVPGHGEEYPAAGAHSSPPKARFLRVPSEHPYLTPSPESPEHWASPSPPSLSDWSESTPSPATATGAMATTTGALPAQPLPLSVPSSLAQAQTQLGPQPEVTPKRQVLA

SEQ ID NO: 9 - DNA, 1998 bp, human
atggtgtacccctatcctggggcccgggctgaagaaaagctaggaggaactcgggaccccacctatcaggagagagcagcccctcaaacgcagcccctgggcaaggagaccgactccctcagtgctgggttcgtggtggtcatgggtgtggatttgtcccgctgtggccctgaccacccggcatcccgctgtccctgggaccctgggcttctactccgcttccttgctgcgatggctgcagtgggagccctggagcccctgctgcctggaccactgctggctgtccaccctcatgcagggaccgcaccccctgccaaccagcttccctggcctgtgctgtgctccccagtggccggggtgattctcctggccctaggggctcttctcgtcctccagctcatccggcgtcgacgccgagagcatggagctctctggctgccccctggtttcactcgacggcctcggactcagtcagctccccaccgacgccggcccccactaggcgaggacagcattggtctcaaggcactgaagccaaaggcagaagttgatgaggatggagttgtgatgtgctcaggccctgaggagggagaggaggtgggccaggctgaagaaacaggcccaccctccacgtgccagctctggtctctgagtggtggctgtggggcgctccctcaggcagccatgctaactcctccccaggaatctgagatggaagcccctgacctggacacccgtggacctgatggggtgacacccctgatgtcagcagtttgctgtggggaagtacagtccgggaccttccaaggggcatggttgggatgtcctgagccctgggaacctctgctggatggaggggcctgtccccaggctcacaccgtgggcactggggagacccccctgcacctggctgcccgattctcccggccaaccgctgcccgccgcctccttgaggctggagccaaccccaaccagccagaccgggcagggcgcacaccccttcatgctgctgtggctgctgatgctcgggaggtctgccagcttctgctccgtagcagacaaactgcagtggacgctcgcacagaggacgggaccacacccttgatgctggctgccaggctggcggtggaagacctggttgaagaactgattgcagcccaagcagacgtgggggccagagataaatgggggaaaactgcgctgcactgggctgctgccgtgaacaacgcccgagccgcccgctcgcttctccaggccggagccgataaagatgcccaggacaacagggagcagacgccgctattcctggcggcgcgggaaggagcggtggaagtagcccagctactgctggggctgggggcagcccgagagctgcgggaccaggctgggctagcgccggcggacgtcgctcaccaacgtaaccactgggatctgctgacgctgctggaaggggctgggccaccagaggcccgtcacaaagccacgccgggccgcgaggctgggcccttcccgcgcgcacggacggtgtcagtaagcgtgcccccgcatgggggcggggctctgccgcgctgccggacgctgtcagccggagcaggccctcgtgggggcggagcttgtctgcaggctcggacttggtccgtagacttggctgcgcgggggggcggggcctattcgcattgccggagcctctcgggagtaggagcaggaggaggcccgacccctcgcggccgtaggttttctgcaggcatgcgcgggcctcggcccaaccctgcgataatgcgaggaagatacggagtggctgccgggcgcggaggcagggtctcaacggatgactggccctgtgattgggtggccctgggagcttgcggttctgcctccaacattccgatcccgcctccttgccttactccgtccccggagcggggatcacctcaacttgactgtggtcccccagccctccaagaaatgcccataaaccaaggaggagagggtaaaaaa

SEQ ID NO: 10 - protein, 666 aa, human
MVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSIGLKALKPKAEVDEDGVVMCSGPEEGEEVGQAEETGPPSTCQLWSLSGGCGALPQAAMLTPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

SEQ ID NO: 11 - DNA, 28 bp, Artificial Sequence
  human primer with restriction site
ggttgtgggtgtcgctagtggatcccgc

SEQ ID NO: 12 - DNA, 28 bp, Artificial Sequence
  human primer with restriciton site
gcgaagcttaccatggtggccgggaccc

SEQ ID NO: 13 - DNA, 20 bp, Artificial Sequence
  primer based on plasmid sequence
ggtgggaggtctatataagc

SEQ ID NO: 14 - DNA, 22 bp, Artificial Sequence
  primer based on plasmid sequence
ggacaaaccacaactagaatgc

SEQ ID NO: 15 - DNA, 25 bp, human
gtgtggggtgaaggccaatgagggc

SEQ ID NO: 16 - DNA, 26 bp, human
tcttccaggtgtcaacgaggtccagg

SEQ ID NO: 17 - DNA, 25 bp, human
gtgcggggtcaaggscaacttccag

SEQ ID NO: 18 - DNA, 25 bp, human
ycacagackggtccagggamgcctg

SEQ ID NO: 19 - DNA, 25 bp, human
aaccgaggtccaagccgcatgtgcc

SEQ ID NO: 20 - DNA, 48 bp, Artificial Sequence
  human primer sequence with restriction site and histidine tag
cgcggatcctaatggtgatggtgatgatgggggaagctgggcggcaag

SEQ ID NO: 21 - DNA, 28 bp, Artificial Sequence
  human primer with restriction site
gggggatccggggaagctgggcggcaag

SEQ ID NO: 22 - DNA, 33 bp, Artificial Sequence
  human primer with restriction site
cccggatcccccaaatcttgtgacaaaactcac

SEQ ID NO: 23 - DNA, 55 bp, Artificial Sequence
  human primer sequence with restriction site and histidine tag
cgctctgagctaatggtgatggtgatgatgtttacccggagacagggagaggctc

SEQ ID NO: 24 - DNA, 35 bp, Artificial Sequence
  human primer with restriction site
ccggaattccatggcaaaacgaaagcgtaagcatg

SEQ ID NO: 25 - DNA, 32 bp, Artificial Sequence
  human primer with restriction site
cgcggatccgtctctacactggaggtggactc

SEQ ID NO: 26 - DNA, 31 bp, Artificial Sequence
  human primer with restriction site
cgcggatcctcaggggaagctgggcggcaag